(19) United States
(12) Patent Application Publication
(10) Pub. No.: US 2024/0301518 A1
(43) Pub. Date: Sep. 12, 2024

(54) MICROBIAL BIOMARKERS IN TRANSPLANTATION AND RELATED METHODS

(71) Applicant: Karius, Inc., Redwood City, CA (US)

(72) Inventors: Sivan Bercovici, Redwood City, CA (US); Kevin Brick, Bethesda, MD (US); Martin S. Lindner, Weinheim (DE); Nicholas Noll, Santa Barbara, CA (US); Gayathri Balasundaram, Sammamish, WA (US)

(21) Appl. No.: 18/610,208

(22) Filed: Mar. 19, 2024

Related U.S. Application Data
(63) Continuation of application No. PCT/US24/17740, filed on Feb. 28, 2024.
(60) Provisional application No. 63/487,592, filed on Feb. 28, 2023.

(51) Int. Cl. C12Q 1/70; C12Q 1/6806; C12Q 1/6874; G16B 30/10; G16H 20/10; G16H 50/20

(52) U.S. Cl. CPC C12Q 1/70; C12Q 1/6806; C12Q 1/6874; G16B 30/10; G16H 20/10; G16H 50/20

(57) ABSTRACT

The present disclosure provides methods and systems for detecting active microbial infection. In some cases, the methods can be used to detect active infection in a recipient of a transplanted organ, graft, or medical device. The present disclosure provides a method of monitoring disease in a human subject, wherein the method comprises obtaining a biological sample from the human subject; measuring concentrations of microbial cell-free nucleic acid (mcfNA) in the biological sample; comparing sequencing coverage near the origin of replication of a microbe to coverage in later replicating regions to derive a peak-to-trough ratio (PTR) score; and based at least on the PTR score, determining a rate of replication of the microbe. The methods described herein may analyze fragment lengths.

### CROSS-REFERENCE

This application is a continuation application of International Patent Application No. PCT/US2024/017740, filed on Feb. 28, 2024, which claims the benefit of U.S. Provisional Patent Application 63/487,592, filed on Feb. 28, 2023, each of which application is incorporated herein by reference in its entirety.

### BACKGROUND

Organ transplantation may be used to treat patients with severe injury or organ failure. Although often lifesaving, organ transplants can also be susceptible to immune-mediated rejection or to infection. Such causes can be difficult to distinguish. In the case of an infection, it can be challenging to distinguish between active and latent infections. For example, increases in viral concentration over time do not necessarily indicate active replication. There is a need for methods that can identify causes of transplant injury; such methods may provide a clearer picture of the role played by an infection, if any, in a transplant injury and could inform treatment. Similarly, there is a need in the art to distinguish between active and latent infection in the context of transplantation, including in the context of xenotransplantation, where the transplanted organ or tissue originates from a species different from the organ or tissue recipient.

### SUMMARY OF THE INVENTION

Aspects disclosed herein include methods for identifying active replication of a microbe in a human subject, wherein the method comprises: providing a biological sample from the human subject, wherein the microbe is present in the human subject or suspected of being present in the human subject; performing massively parallel sequencing on cell-free nucleic acids (cfNA) in the biological sample to generate microbial cell-free nucleic acid (mcfNA) sequence reads; aligning the mcfNA sequence reads with a reference sequence containing sequences within an origin of replication region of the microbial genome to determine coverage of the mcfNA within the origin of replication region of the microbial genome; and determining whether the microbe is actively replicating in the human subject based on the coverage of the mcfNA within the origin of replication region of the microbial genome. In some embodiments, the method further comprises comparing the coverage of the mcfNA within the origin of replication region of the microbial genome to coverage in later replicating regions to derive a peak-to-trough ratio (PTR) score. In some embodiments, the determining in (d) comprises using the PTR score to determine whether the microbe is actively replicating in the human subject. In some embodiments, the massively parallel sequencing is whole genome sequencing. In some embodiments, the massively parallel sequencing is Next Generation Sequencing. In some embodiments, the Next Generation Sequencing is Next Next Generation sequencing. In some embodiments, the method further comprises determining a concentration or quantity of the mcfNA. In some embodiments, the method further comprises monitoring the concentration or quantity of the mcfNA over time. In some embodiments, the method further comprises identifying fragments of mcfNA that vary during a course of treatment. In some embodiments, the fragments of mcfNA are within the origin of replication region of the microbial genome and span the origin of replication or are within the origin of replication region but do not span the origin of replication. In some embodiments, sequences within the origin of replication region of the microbial genome are within 0.5 kb, 1 kb, 1.5 kb, 2 kb, 5 kb, 8 kb, 10 kb, 15 kb, 20 kb, 25 kb, 30 kb or 50 kb of either side of the origin of replication and wherein the sequences either span or do not span the origin of replication. In some embodiments, the later replicating region is a region greater than 50 kb, 75 kb, 100 kb, or 150 kb from either side of the origin of replication. In some embodiments, the later replicating region is a region within 0.5 kb, 1 kb, 1.5 kb, 2 kb, 5 kb, 8 kb, 10 kb, 15 kb, 20 kb, 25 kb, 30 kb or 50 kb of one or more replication termini and wherein the later replicating region either spans or does not span a replication terminus. In some embodiments, active replication is identified when coverage within the origin of replication region of the microbial genome and coverage at a later replicating region area statistically different from each other and wherein the coverage at the origin of replication region of the microbial genome is higher compared to the coverage at the later replicating regions. In some embodiments, the PTR score is a statistically significant score and wherein active replication is identified when the PTR score is greater than 1. In some embodiments, the PTR score is a statistically significant score and wherein active replication is identified when the PTR score is greater than 1.2. In some embodiments, the PTR score is a statistically significant score and coverage at the origin of replication region of the microbial genome is greater than two reads and the coverage at the later replicating region is greater than two reads. In some embodiments, active replication is monitored by detecting statistically significant PTR scores over time. In some embodiments, a worsening infection is identified when a magnitude of statistically significant PTR scores increases over a period of at least two days. In some embodiments, a worsening infection is identified when a magnitude of statistically significant PTR scores increases over a period of at least five days. In some embodiments, a worsening infection is identified when a magnitude of statistically significant PTR scores increases by at least two-fold over a period of 10 days. In some embodiments, a worsening infection is identified when the PTR score increases in a statistically significant manner over a period of at least 5 days. In some embodiments, the human subject is a transplant recipient. In some embodiments, the human subject is a xenotransplant recipient. In some embodiments, the human subject is a transplant recipient of a non-human mammalian organ or graft, a pig organ or graft, or a bovine organ or graft. In some embodiments, the xenotransplant is selected from the group consisting of a pig heart, a pig liver, a pig lungs, pig kidney, bovine kidney, bovine liver, bovine lungs, non-human mammalian heart, non-human mammalian liver, and non-human mammalian lungs. In some embodiments, the microbe is a bacterium or virus associated with a transplanted organ or graft or with a xenotransplanted organ or graft. In some embodiments, the microbe is a virus. In some embodiments, the virus is a DNA virus. In some embodiments, the virus is an enveloped virus. In some embodiments, the microbe is a virus selected from EBV, CMV, HSV, and HTLV. In some embodiments, the virus is a double-stranded DNA virus. In some embodiments, the virus is in the order Herpesvirales. In some embodiments, the virus is a cytomegalovirus (CMV). In some embodiments, the virus is porcine CMV (pCMV). In some embodiments, the cell-free nucleic acids are cfDNA. In some embodiments, the mcfNA comprises microbial DNA. In some embodiments, the mcfNA comprises microbial RNA. In some embodiments, the measuring concentrations of mcfNA in the biological sample comprises preparing a nucleic acid library. In some embodiments, the method further comprises measuring lengths of nucleic acid fragments at the origin of replication region of the microbial genome. In some embodiments, the method further comprises determining gene strand bias of the bottom and top strands at the origin of replication region of the microbial genome. In some embodiments, the method further comprises measuring fluctuations in fragment populations over time following treatment. In some embodiments, the method further comprises measuring fluctuations in the amount of ˜55 nt or ˜140 nt fragments of mcfDNA. In some embodiments, the method further comprises measuring relative abundance of reads from ˜55 nt population of microbial mcfNA. In some embodiments, the method further comprises measuring fluctuations in the amount of mcfDNA from short, ssDNA-derived mcfDNA fragments. In some embodiments, the method further comprises producing and sequencing DNA libraries, and aligning sequence reads to a reference genome of the transplant. In some embodiments, the method further comprises aligning sequence reads to a genome of a microbe. In some embodiments, the method further comprises aligning sequence reads to a genome of a bacterium. In some embodiments, the method further comprises aligning sequence reads to a genome of a virus. In some embodiments, the method further comprises aligning sequence reads to a CMV genome. In some embodiments, the method further comprises measuring principal components (PCs) of longest contiguous reads. In some embodiments, the method further comprises comparing PCs to a PTR score. In some embodiments, the method further comprises identifying a disease, disorder, or condition in the human subject wherein the disease, disorder, or condition is selected from the group consisting of: xenograft injury, transplant rejection, active viral infection, and latent viral infection. In some embodiments, the method further comprises administering a treatment to the human subject to treat a microbial infection identified by the method. In some embodiments, the treatment is an antiviral drug. In some embodiments, the method further comprises monitoring a response to the treatment, wherein the response to the treatment is monitored by performing massively parallel sequencing on cell-free nucleic acids (cfNA) in a biological sample collected after the administering a treatment to the human subject in order to generate microbial cell-free nucleic acid (mcfNA) sequence reads; aligning the mcfNA sequence reads with a reference sequence containing sequences near an origin of replication of the microbe to determine coverage of the mcfNA near the origin of replication; and determining whether the microbe is actively replicating in the human subject based on the coverage of the mcfNA near the origin of replication. In some embodiments, the antiviral drug is an antiviral, an abacavir, an acyclovir (aciclovir), an adefovir, an amantadine, an ampligen, an amprenavir (agenerase), an umifenovir (arbidol), an atazanavir, an atripla (efavirenz/emtricitabine/tenofovir), a baloxavir marboxil (xofluza), a biktarvy (bictegravir/emtricitabine/tenofovir alafenamide), a boceprevir, a bulevirtide, a cidofovir, a cobicistat (tybost), a combivir (lamivudine/zidovudine), a daclatasvir (daklinza), a darunavir, a delavirdine, a descovy (emtricitabine/tenofovir alafenamide), a didanosine, a docosanol, a dolutegravir, a doravirine (pifeltro), an edoxudine, an efavirenz, an elvitegravir, an emtricitabine, an enfuvirtide, an ensitrelvir, an entecavir, an etravirine (intelence), a famciclovir, a fomivirsen, a fosamprenavir, a foscamet, a ganciclovir (cytovene), an ibacitabine, an ibalizumab (trogarzo), an idoxuridine, an imiquimod, an inosine pranobex, an indinavir, a lamivudine, a letermovir (prevymis), a lopinavir, a loviride, a maraviroc, a methisazone, a moroxydine, a nelfinavir, a nirmatrelvir/ritonavir (paxlovid), a nevirapine, a nitazoxanide, a norvir, an oseltamivir (tamiflu), a penciclovir, a peramivir, a penciclovir, a peramivir (rapivab), a pleconaril, a podophyllotoxin, a raltegravir, a remdesivir, a ribavirin, a rilpivirine (edurant), a rilpivirine, a rimantadine, a ritonavir, a saquinavir, a simeprevir (olysio), a sofosbuvir, a stavudine, a taribavirin (viramidine), a telaprevir, a telbivudine (tyzeka), a tenofovir alafenamide, a tenofovir disoproxil, a tipranavir, a trifluridine, a trizivir, a tromantadine, a truvada, an umifenovir, a valaciclovir (valtrex), a valganciclovir (valcyte), a vicriviroc, a vidarabine, a zalcitabine, a zanamivir (relenza), a zidovudine, a stereoisomer of any of these, a salt of any of these, or any combination thereof. In some embodiments, the method further comprises detecting donor-derived cell-free DNA in the transplant recipient in order to detect transplant rejection. In some embodiments, the method further comprises administering an immunosuppressant drug to the transplant recipient to treat the transplant rejection. In some embodiments, the biological sample is a biological fluid sample. In some embodiments, the biological fluid sample is a cell-free biological fluid sample. In some embodiments, the biological fluid sample is a plasma sample, a serum sample, a blood sample, a saliva sample, a synovial fluid sample, a cerebrospinal fluid sample, or a urine sample. In some embodiments, the biological fluid sample is a plasma sample.

Aspects disclosed herein include methods for using a quantity of sequence reads at the origin of replication region of the microbial genome to determine whether a subject has an active infection, the method comprising: providing a plasma or serum sample comprising microbial cell free nucleic acids from a subject; performing massively parallel sequencing on the microbial cell-free nucleic acids to obtain sequence reads associated with a microbe; detecting a coverage bias of sequence reads covering a genomic locus at the origin of replication region of the microbial genome; and determining whether there is active replication of the microbe at least in part from the presence or absence of the coverage bias, wherein the active replication of the microbe is associated with an infection of an internal solid organ. In some embodiments, the median of the fragment lengths is between 30-80 nt, 40-65 nt, or 45-70 nt.

Aspects disclosed herein include methods for detecting whether an organ transplant recipient has an active infection, the method comprising: providing sequence data from the organ transplant recipient, wherein the sequence data comprises sequence reads created from sequencing a sample from the organ transplant recipient, wherein the sample comprises a plurality of microbial cell-free DNA fragments of different lengths; analyzing the sequence data to determine a profile of the microbial cell-free DNA fragments of different lengths; modeling the microbial cell-free DNA fragments as a set of normal distributions with medians at a first fragment length and a second fragment length, wherein the first fragment length is shorter than the second fragment length; and identifying an active infection when the normal distribution with the median at the first fragment length constitutes greater than 20% of all fragments of microbial cell-free DNA in the sample. In some embodiments, the normal distribution with the median at the first fragment length has a median fragment length of about 55 nt. In some embodiments, the normal distribution with the median at the second fragment length has a median fragment length of about 140 nt. In some embodiments, the method further comprises identifying a normal distribution with a median at a third fragment length, wherein the third fragment length is about 90 nt. In some embodiments, the normal distribution with the median at the first fragment length has a median fragment length of at least 30 nt. In some embodiments, the normal distribution with the median at the first fragment length has a median fragment length of at most 80 nt. In some embodiments, the normal distribution with the median at the first fragment length has a median fragment length of at least 40 nt. In some embodiments, the normal distribution with the median at the first fragment length has a median fragment length of at most 65 nt. In some embodiments, an active infection is identified when the normal distribution with the median at the first fragment length constitutes greater than 30% of all fragments of microbial cell-free DNA in the sample. In some embodiments, an active infection is identified when the normal distribution with the median at the first fragment length constitutes greater than 50% of all fragments of microbial cell-free DNA in the sample. In some embodiments, the median of the fragment lengths is between 30-80 nt, 40-65 nt, or 45-70 nt.

Aspects disclosed herein include methods for detecting whether an organ transplant recipient has an active infection, the method comprising: providing sequence data from the organ transplant recipient, wherein the sequence data comprises sequence reads created from sequencing a sample from the organ transplant recipient, wherein the sample comprises a plurality of microbial cell-free DNA fragments of different lengths; analyzing the sequence data to determine a profile of the microbial cell-free DNA fragments of different lengths; modeling the microbial cell-free DNA fragments as a composite of three distinct Gaussian distributions predetermined to have median lengths of about 55 nt, about 90 nt, and about 140 nt; and identifying an active infection when the Gaussian distribution with the median length of 55 nt constitutes greater than 20% of all fragments of microbial cell-free DNA in the sample. In some embodiments, the median of the fragment lengths is between 30-80 nt, 40-65 nt, or 45-70 nt.

Aspects disclosed herein include methods for detecting an actively replicating microbe in a human subject, the method comprising: providing a biological sample comprising microbial cell-free nucleic acids from a human subject; contacting the microbial cell-free nucleic acids with primers specific for the replicating microbe, wherein the primers are designed to produce amplicons that span at least a portion of an origin of replication region of a microbial genome; and subjecting the microbial cell-free nucleic acids to a quantitative polymerase chain reaction (qPCR) using the primers specific for the replicating microbe, thereby detecting actively replicating microbe in the human subject. In some embodiments, the nucleic acids are cell-free DNA. In some embodiments, the actively replicating virus is CMV. In some embodiments, the method further comprises comparing (i) the coverage of the plurality of sequence reads for the origin of replication region to (ii) the coverage of the plurality of sequence reads for later replicating regions to derive a peak-to-trough ratio (PTR) score. In some embodiments, the median of the fragment lengths is between 30-80 nt, 40-65 nt, or 45-70 nt.

Aspects disclosed herein include methods for detecting an alteration in a prosthetic device that has been implanted in a subject, the method comprising: providing a biological sample from a subject; sequencing cell-free nucleic acids (cfNA) from the biological sample to generate sequence data comprising a plurality of sequence reads for microbial cell-free DNA fragments; processing the sequence data to determine a coverage of the plurality of sequence reads for an origin of replication region and a coverage of the plurality of sequence reads for a later replicating region; and based at least on the coverage of the plurality of sequence reads for the origin of replication region and the coverage of the plurality of sequence reads for the later replicating region, identifying an alteration in the prosthetic device, wherein the alteration in the prosthetic device is a microbial infection associated with the prosthetic device. In some embodiments, the method further comprises comparing (i) the coverage of the plurality of sequence reads for the origin of replication region to (ii) the coverage of the plurality of sequence reads for a later replicating region to derive a peak-to-trough ratio (PTR) score. In some embodiments, the method further comprises administering a treatment to the human subject to treat the microbial infection associated with the prosthetic device. In some embodiments, the microbial infection is a bacterial infection. In some embodiments, the treatment is an antibacterial drug or antibiotic. In some embodiments, the method further comprises monitoring a response to the treatment, wherein the response to the treatment is monitored by performing massively parallel sequencing on cell-free nucleic acids (cfNA) in a biological sample collected after the administering a treatment to the human subject in order to generate microbial cell-free nucleic acid (mcfNA) sequence reads; aligning the mcfNA sequence reads with a reference sequence containing sequences within an origin of replication region of the microbial genome to determine coverage of the mcfNA within the origin of replication region; and determining whether the microbe is actively replicating in the human subject based on the coverage of the mcfNA within the origin of replication region of the microbial genome. In some embodiments, the microbe is a Staphylococcus bacterium or Nocardia bacterium. In some embodiments, the prosthetic device is made entirely of non-biological materials. In some embodiments, the prosthetic device is partially made of biological material and partially made of non-biological materials. In some embodiments, the prosthetic device is a prosthetic heart valve. In some embodiments, the prosthetic device is a prosthetic heart valve that comprises porcine tissue. In some embodiments, the prosthetic device comprises a metal, titanium, cobalt, pyrolytic carbon, or a polymer.

Aspects disclosed herein include methods for detecting an alteration in a prosthetic device that has been implanted in a subject, the method comprising: providing a biological sample from the subject; sequencing cell-free nucleic acids (cfNA) from the biological sample to generate sequence data, wherein the sequence data comprises a plurality of sequence reads for microbial cell-free DNA fragments of different lengths; processing the sequence data to determine a profile of microbial cell-free DNA fragment lengths in the sample; and based at least on the profile of microbial cell-free DNA fragment lengths in the sample, identifying an alteration in the prosthetic device, wherein the alteration is a microbial infection associated with the prosthetic device. In some embodiments, the method further comprises modeling the microbial cell-free DNA fragments as a set of normal distributions with medians at a first fragment length and a second fragment length, wherein the first fragment length is shorter than the second fragment length; and identifying an active infection when the normal distribution with the median at the first fragment length constitutes greater than 20% of all fragments of microbial cell-free DNA in the sample. In some embodiments, the median at the first fragment length is about 55 nt. In some embodiments, the median at the second fragment length is about 140 nt. In some embodiments, the method further comprises identifying a normal distribution with a median at a third fragment length, wherein the third fragment length is about 90 nt. In some embodiments, the method further comprises administering an antibiotic drug to the subject when actively replicating bacteria are detected. In some embodiments, the prosthetic device is made entirely of non-biological materials. In some embodiments, the prosthetic device is partially made of biological material and partially made of non-biological materials. In some embodiments, the prosthetic device is a prosthetic heart valve. In some embodiments, the prosthetic device is a prosthetic heart valve that comprises porcine tissue. In some embodiments, the prosthetic device comprises a metal, titanium, cobalt, pyrolytic carbon, or a polymer.

Disclosed herein in some embodiments, is a method of identifying active replication of a microbe in a human subject. In some embodiments, a method can comprise providing a biological sample from a human subject. In some embodiments, a microbe can be present in a human subject or suspected of being present in a human subject. In some embodiments, a method can comprise generating sequence reads associated with microbial cell-free nucleic acids (mcfNA) from a biological sample by performing massively parallel sequencing on cell-free nucleic acids in a biological sample. In some embodiments, a method can comprise aligning a sequence reads corresponding to sequences associated with microbial cell-free nucleic acids (mcfNA) with a reference sequence containing sequences near an origin of replication of a microbe to determine coverage of a mcfNA near an origin of replication. In some embodiments, a method can comprise determining whether a microbe is actively replicating in a human subject based on a coverage of a mcfNA near an origin of replication. In some embodiments, a method can comprise comparing a coverage of a mcfNA near an origin of replication of a microbe to coverage in later replicating regions to derive a peak-to-trough ratio (PTR) score. In some embodiments, a determining can comprise using a PTR score to determine whether a microbe is actively replicating in a human subject. In some embodiments, a massively parallel sequencing can comprise whole genome sequencing. In some embodiments, a massively parallel sequencing can comprise Next Generation Sequencing. In some embodiments, a Next Generation Sequencing can comprise a Next Next Generation sequencing. In some embodiments, a method can comprise determining a concentration or quantity of a mcfNA. In some embodiments, a method can comprise monitoring a concentration or quantity of a mcfNA over time. In some embodiments, a method can comprise identifying fragments of mcfNA that vary during a course of treatment. In some embodiments, fragments of mcf NA are near an origin of replication. In some embodiments, sequences near an origin of replication are within 0.5 kb, 1 kb, 1.5 kb, 2 kb, 5 kb, 8 kb, 10 kb, 15 kb, 20 kb, 25 kb, 30 kb or 50 kb of an origin of replication. In some embodiments, later replicating regions are regions greater than 50 kb, 75 kb, 100 kb, or 150 kb from an origin of replication. In some embodiments, a PTR ratio does not include coverage at positions 20 kb-30 kb or 150 kb-160 kb. In some embodiments, a human subject can be a transplant recipient. In some embodiments, a human subject can be a xenotransplant recipient. In some embodiments, a human subject can be a transplant recipient of a pig organ or graft. In some embodiments, a xenotransplant can be selected from a pig heart, pig liver, pig lungs, or pig kidney. In some embodiments, a microbe can comprise a CMV from a xenotransplant. In some embodiments, a microbe can comprise a virus. In some embodiments, a virus can comprise a DNA virus. In some embodiments, a virus can comprise an enveloped virus. In some embodiments, a microbe can comprise a virus selected from EBV, CMV, HSV, and HTLV. In some embodiments, a virus can comprise a double-stranded DNA virus. In some embodiments, a virus can be in an order Herpesvirales. In some embodiments, a virus can comprise a cytomegalovirus (CMV). In some embodiments, a virus can comprise a porcine CMV (pCMV). In some embodiments, a cell-free nucleic acid can comprise cfDNA. In some embodiments, a mcfNA can comprise microbial DNA. In some embodiments, a mcfNA can comprise microbial RNA. In some embodiments, measuring concentrations of mcfNA in a biological sample can comprise preparing a nucleic acid library. In some embodiments, determining a quantity or concentration of mcfNA in a biological sample can comprise next generation sequencing of mcfNA. In some embodiments, a method can comprise measuring lengths of nucleic acid fragments near an origin of replication. In some embodiments, a method can comprise determining gene strand bias of a bottom and a top strand near an origin of replication. In some embodiments, a method can comprise measuring fluctuations in fragment populations over time following treatment. In some embodiments measuring fluctuations in an amount of ˜55 nt or ˜140 nt fragments of mcfDNA. In some embodiments, measuring relative abundance of reads from ˜55 nt population of microbial mcfNA. In some embodiments, measuring fluctuations in an amount of mcfDNA from short, ssDNA-derived mcfDNA fragments. In some embodiments, producing and sequencing DNA libraries, and aligning sequence reads to a reference genome of a donor organ. In some embodiments, aligning sequences to genome of microbes derived from a donor organ. In some embodiments, aligning sequences to a genome of a virus. In some embodiments, aligning sequences to a CMV genome derived from a donor organ. In some embodiments, a method can further comprise limiting reads that had at least 4000 reads. In some embodiments, a method can further comprise measuring principal components (PCs) of longest contiguous reads. In some embodiments, a method can further comprise comparing PCs to PTR. In some embodiments, a method can further comprise identifying a disease, disorder or condition in a human subject wherein a disease, disorder or condition can be selected from a group consisting of xenograft injury, transplant rejection, active viral infection and latent viral infection. In some embodiments, a method can further comprise administering a treatment to a human subject. In some embodiments, a treatment can comprise an antiviral drug. In some embodiments, a method can further comprise detecting mcfDNA coverage around a viral origin of replication as an indication of response to a treatment. In some embodiments, an antiviral drug can comprise an antiviral, an abacavir, an acyclovir (aciclovir), an adefovir, an amantadine, an ampligen, an amprenavir (agenerase), an umifenovir (arbidol), an atazanavir, an atripla (efavirenz/emtricitabine/tenofovir), a baloxavir marboxil (xofluza), a biktarvy (bictegravir/emtricitabine/tenofovir alafenamide), a boceprevir, a bulevirtide, a cidofovir, a cobicistat (tybost), a combivir (lamivudine/zidovudine), a daclatasvir (daklinza), a darunavir, a delavirdine, a descovy (emtricitabine/tenofovir alafenamide), a didanosine, a docosanol, a dolutegravir, a doravirine (pifeltro), an edoxudine, an efavirenz, an elvitegravir, an emtricitabine, an enfuvirtide, an ensitrelvir, an entecavir, an etravirine (intelence), a famciclovir, a fomivirsen, a fosamprenavir, a foscamet, a ganciclovir (cytovene), an ibacitabine, an ibalizumab (trogarzo), an idoxuridine, an imiquimod, an inosine pranobex, an indinavir, a lamivudine, a letermovir (prevymis), a lopinavir, a loviride, a maraviroc, a methisazone, a moroxydine, a nelfinavir, a nirmatrelvir/ritonavir (paxlovid), a nevirapine, a nitazoxanide, a norvir, an oseltamivir (tamiflu), a penciclovir, a peramivir, a penciclovir, a peramivir (rapivab), a pleconaril, a podophyllotoxin, a raltegravir, a remdesivir, a ribavirin, a rilpivirine (edurant), a rilpivirine, a rimantadine, a ritonavir, a saquinavir, a simeprevir (olysio), a sofosbuvir, a stavudine, a taribavirin (viramidine), a telaprevir, a telbivudine (tyzeka), a tenofovir alafenamide, a tenofovir disoproxil, a tipranavir, a trifluridine, a trizivir, a tromantadine, a truvada, an umifenovir, a valaciclovir (valtrex), a valganciclovir (valcyte), a vicriviroc, a vidarabine, a zalcitabine, a zanamivir (relenza), a zidovudine, a stereoisomer of any of these, a salt of any of these, or any combination thereof. In some embodiments, a method can further comprise determining that a transplant recipient has a rejection of an organ, further comprising treating an organ transplant recipient with an immunosuppressant. In some embodiments, a biological sample can comprise a biological fluid sample. In some embodiments, a biological fluid sample can comprise a cell-free biological fluid sample. In some embodiments, a biological fluid sample can comprise a plasma sample, serum sample, blood sample, saliva sample, synovial fluid sample, cerebrospinal fluid sample, or urine sample. In some embodiments, a biological fluid sample can comprise a plasma sample.

Disclosed herein in some embodiments is a method of using a quantity of sequence reads near an origin of replication to determine whether a subject has an active infection. In some embodiments, a method can further comprise providing a sample containing microbial cell free nucleic acids from a subject. In some embodiments, a method can further comprise performing massively parallel sequencing on microbial cell-free nucleic acids to obtain sequence reads associated with a microbe. In some embodiments, detecting a coverage bias of sequence reads covering a genomic loci near an origin of replication of a microbe. In some embodiments, a method can further comprise determining whether there is active replication of a microbe at least in part from a presence or absence of a coverage bias.

Disclosed herein in some embodiments is a method of detecting whether an organ transplant recipient has an infection or an organ rejection. In some embodiments, a method can further comprise providing sequence data from an organ transplant recipient. In some embodiments, sequence data comprises sequence reads created from sequencing a sample from an organ transplant recipient. In some embodiments, a sample comprises a plurality of cell-free DNA fragments of different lengths. In some embodiments, a method can further comprise analyzing a sequence data to determine a profile of DNA fragment lengths in a sample. In some embodiments, if a fragment length profile shows a highest peak of fragment lengths around 55 bp then determining that there is active viral replication in an organ transplant recipient. In some embodiments, a method can comprise determining that an organ transplant recipient has a rejection of an organ if a fragment length profile does not show a highest peak of fragment lengths around 55 bp.

Disclosed herein in some embodiments is a method of detecting whether an organ transplant recipient has an infection or an organ rejection. In some embodiments, a method can comprise providing a sample from an organ transplant recipient. In some embodiments, a method can comprise performing high-throughput sequencing on a sample from an organ transplant recipient to generate sequence data. In some embodiments, a sample comprises a plurality of cell-free DNA fragments of different lengths. In some embodiments, a method can comprise analyzing a sequence data to determine a profile of DNA fragment lengths in a sample. In some embodiments, a method can comprise determining that there is active viral replication in an organ transplant recipient if a fragment length profile shows a highest peak of fragment lengths around 55 bp. In some embodiments, a method can comprise determining that an organ transplant recipient has a rejection of an organ if a fragment length profile shows a highest peak of fragment lengths around 147 bp.

Disclosed herein in some embodiments is a method of detecting actively replicating virus in a human subject. In some embodiments, a method can comprise providing a biological sample from a human subject. In some embodiments, a method can comprise extracting nucleic acids from a biological sample from a human subject. In some embodiments, a method can comprise contacting an extracted nucleic acids with primers specific for a replicating virus. In some embodiments, a primers can be designed to produce amplicons that span an origin of replication for a virus or that are less than 50 base pairs in length. In some embodiments, a method can comprise subjecting an extracted nucleic acids to a quantitative polymerase chain reaction (qPCR) using primers specific for a replicating virus, thereby detecting actively replicating virus in a human subject. In some embodiments, nucleic acids are cell-free DNA. In some embodiments, an actively replicating virus can comprise CMV.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties and to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-E show pCMV replication is elevated late in treatment. FIG. 1A shows treatment regimen for xenotransplant patient. FIG. 1B shows the pCMV (porcine cytomegalovirus) PTR peaks at day 53. PTR can be heavily impacted by varying genomic coverage; the range of PTR values expected from stochastic coverage variation for each sample was inferred by calculating PTR values for 1,000 coverage profiles where genome positions were randomly shuffled (indicated by median and error bars). FIG. 1C shows relative quantification of human, porcine and pCMV cfDNA. MPMs are expressed as a multiple of the MPM at day 20, when pCMV was first detected. The normalized results illustrate that the rate of increase of pCMV concentration greatly exceeds the rates for porcine-derived and human-derived cfDNA. FIG. 1D shows quantification of the magnitude of mcfDNA strand asymmetry around the pCMV origin of replication. Circles depict the mean enriched strand/total coverage in 20 Kb windows (error bars show 95% confidence intervals). FIG. 1E shows reciprocal fluctuations in the amount of mcfDNA from ˜140 nt fragments and from short, likely ssDNA-derived mcfDNA fragments. The fluctuations for shorter pCMV fragments correlate tightly with changes in pCMV PTRs shown in FIG. 1B.

FIGS. 2A-D show pCMV PTRs correlate with the abundances of different fragment populations. FIG. 2A shows correlations between pCMV PTRs and cfDNA concentrations in plasma. The left panels show the correlation between values at the same time point (Actual). In the right panels, PTR values are instead compared to the MPM from the subsequent time point (Offset; corresponds to 6/7 days; see FIG. 1A). FIG. 2B shows PTR values are correlated between the dsDNA-only (KT) and ssDNA (KD) protocols. FIG. 2C shows the extent of sequencing read asymmetry is not correlated with the inferred pCMV PTRs. It is negatively correlated with pCMV cfDNA concentration. FIG. 2D shows pCMV PTRs are positively correlated with the relative abundance of reads from the ˜55 nt population. Porcine CMV PTRs are negatively correlated with the relative abundance of reads from the ˜140 nt population. R²=squared Pearson correlation coefficients.

FIGS. 3A-G show microbial fragmentomics reveal signatures of pCMV replication. FIG. 3A shows pCMV cfDNA strand asymmetry flanks the viral origin of replication. FIG. 3B shows this asymmetry is not seen when the reads from a dsDNA-only protocol were examined for sequencing cfDNA. FIG. 3C shows the magnitude of strand asymmetry is similar across time-points and across the genome. Here, coverage is split by enriched/depleted strands to the left/right of an origin and smoothed over 500 nt windows using LOESS regression. The central dip is likely from difficulties in mapping reads at an origin. FIG. 3D shows strand asymmetry does not depend on gene orientation. FWD/REV asymmetry was quantified for every pCMV gene (indicated as “>” or “<” symbols for the positive strand and negative strand genes, respectively). FIG. 3E shows the multi-modal fragment length profile for pCMV (top), human (middle), and porcine cfDNA is accurately reconstructed from a mixture of four populations of fragments. FIG. 3F and FIG. 3G shows pCMV reads from the ˜55 nt and ˜140 nt populations vary in frequency among samples. The fraction of reads attributed to each population is shown at each position. Window size in FIG. 3F is 1 Kb; in FIG. 3G is 100 bp. FIG. 3G shows highly localized changes in fragment lengths at an origin of replication.

FIG. 4 shows gene strand bias in the pCMV genome. The linear dsDNA pCMV genome coordinates are represented on the x-axis. The position of the pCMV origin of replication is indicated with a dashed line. There is a strong bias for bottom-strand genes to the left of the replication origin (left-hand side of figure). Most top-strand genes are to the right of the origin, however, there is a less pronounced top/bottom bias on this side. Each gene is indicated as a dot (+ strand, − strand). Bars indicate the number of top-bottom strand genes in a 12 Kb window.

FIG. 5 shows fluctuations in fragment populations over time. Each position shows the fraction of reads attributed to each population at that position. Window size=1 Kb.

FIGS. 6A-D show window protection scores for the 55 nt and 140 nt pCMV mcfDNA fractions. The window protection score (WPS) is a metric that compares the number of fragments with an endpoint within a genomic window to the number of fragments that completely span the window. Higher values indicate loci that are more protected from fragmentation. FIG. 6A shows the calculated WPS score using fragments of length 55±11 nt (WPS window=55 bp) for the full genome. FIG. 6B shows the calculated WPS score using fragments of length 55±11 nt (WPS window=55 bp) for a ˜25 Kb region centered at the pCMV origin of replication. FIG. 6C shows the calculated WPS score using fragments of length 140±28 nt population (WPS window=140 bp) for the full genome. FIG. 6D shows the calculated WPS score using fragments of length 140±28 nt population (WPS window=140 bp) for a ˜25 Kb region centered at the pCMV origin of replication. The most notable feature observed is a peak in the WPS 140 bp score at the pCMV origin of replication at several time points.

FIGS. 7A-F show biases in coverage patterns surrounding viral and bacterial origins of replication observed in KT clinical samples. FIG. 7A, FIG. 7B, FIG. 7C, and FIG. 7D show the principal components exhibiting the most pronounced peak structure across four different viruses. FIG. 7E and FIG. 7F show coverage profiles of two bacteria with circular genomes, Staphylococcus aureus and Nocardiafarcinica, as studied in two independent cases, respectively. The vertical lines highlight the approximate location of the origin of replication.

FIGS. 8A-D show HHV-5 ORI peak analysis. FIG. 8A shows peak-shaped HHV-5 PC 0 weight is strongly correlated with PTR in 300 clinical samples (Spearman r=0.820, p=2.54e-74).

FIG. 8B shows HHV-5 ORI peak size is not correlated with HHV-5 concentration in 300 clinical samples (Spearman r=0.019, p=0.73). FIG. 8C shows the magnitude of HHV-5 ORI peak in the first and second samples of 74 HHV-5-positive sample pairs taken<14 days apart, showing significantly larger ORI peaks in pairs with decreasing MPM (first: p=0.0058, second: p=0.0003). FIG. 8D shows sample pairs with decreasing HHV-5 MPM tend to have an elevated PTR (higher replication signal) while pairs with increasing MPM tend to have very little PTR change.

FIG. 9 depicts a computer control system that is programmed or otherwise configured to implement the methods and systems provided herein.

### DETAILED DESCRIPTION

### General Overview

This disclosure provides methods and systems that detect cell-free nucleic acids (e.g., cell-free DNA, microbial cell-free DNA (mcfDNA)) in order to identify active microbial replication in a subject. Often, the methods are useful for a transplant recipient or a recipient of a transplanted medical device. In some cases, the methods are used to detect active microbial replication in a subject that has not received a transplanted organ, tissue or device, or to detect an active infection that is present in the subject and not associated with a transplant or implant. In some cases, the cell-free nucleic acids may be detected in samples obtained from a body fluid of the subject or recipient, such as plasma. The methods and systems are useful to detect active infection in xenotransplant recipients; but they can also be used to detect active infection in other transplant recipients, such as recipients of allografts. In some embodiments, the infection is a bacterial or viral infection. In some embodiments, the infection is caused by a virus (e.g., cytomegalovirus (CMV), porcine CMV). In some cases, the methods detect cfDNA fragmentation patterns that can be used as a biomarker for an infection, or biomarker for an active replicating microbe. In some cases, this disclosure concerns detection of particular sequences of cfDNA, such as sequences derived from loci near an origin of replication (ORI), particularly a microbial origin of replication such as a viral origin of replication or a bacterial origin of replication. The methods include detection of temporal changes in mcfDNA coverage around an origin of replication as well as the detection of the emergence of shorter mcfDNA fragments over time.

In some embodiments, whole genome sequencing coverage near the origin of replication is compared to the coverage in later-replicating regions in order to detect an actively replicating microbe (e.g., virus or bacterium). In many prokaryotic and viral species, DNA replication initiates at a single locus in the genome, known as the origin of replication (ORI). From the ORI, replication can proceed bidirectionally outward until the two replication forks meet (for circular chromosomes) or until each replication fork reaches the end of the genome (for linear chromosomes). These ends, in either case can be referred to as a terminus (ter). Because of this ORI-ter structure, the DNA close to the ORI can be present in multiple copies in a population of actively dividing cells. Therefore, when more sequences near the ORI are captured using the methods disclosed herein as compared to the sequences near the ter, the microbe is more likely to be actively replicating. This ORI-ter coverage imbalance can be used to predict bacterial growth rates or viral replication from mcfDNA using both whole genome sequencing assays or PCR (standard PCR, qPCR, RT-PCT). For microbes with a single origin of replication (such as pCMV) the ratio of observed sequencing reads near the origin of replication to those near the terminus, denoted as a Peak (origin) to Trough Ratio (PTR), can provide an estimate of the replication rate. This disclosure also provides methods to detect active replication of a microbe by detecting fragments of the particular microbe, wherein the presence of a certain quantity of such fragments, or a relative increase in such fragments over time, is an indication of active replication. Often, the informative fragments are relatively short (e.g., about 55 nt) compared to other fragments in the sample. The analysis for this particular method may be agnostic to the region of the ORI or to the region around the termini, and rather focuses on the lengths of fragments of mcfDNA (e.g., mdfDNA from a particular microbe, e.g., pCMV). Such a method may rely primarily on the length of fragments associated with a specific microbe rather than the relative quantity of fragments aligned to the ORI or terminus.

The methods provided herein can be useful in the context of xenotransplantation, e.g., in the context of a human subject who is a recipient of a xenotransplant (e.g., a porcine heart). In some embodiments, the method comprises detecting mcfDNA (e.g., pCMV mcfDNA) and cfDNA from the transplanted tissue (e.g., xenograft cfDNA, porcine cfDNA). For example, relatively high concentrations of pCMV mcfDNA and porcine cfDNA may be observed in a body fluid such as plasma. This can, in some cases, occur after withdrawal of a support or therapy to a recipient of a transplanted tissue (e.g., recipient of a porcine heart). Without wishing to be bound by theory, the dominant component of cfDNA can arise from the genomes of apoptotic and necrotic cells (REF).

In some cases, the methods may involve determining that elevated porcine cfDNA is likely a reflection of increased cell death of the transplanted tissue. In some embodiments, the methods distinguish between (a) latent infections (e.g., latent infections exposed as the transplanted tissue fails) and (b) an active microbial infection (e.g., pCMV infection). In some embodiments, the methods include determining that elevated mcfDNA (e.g., pCMV mcfDNA) may have been derived from (a) latent infections that were exposed as the transplanted tissue failed, (b) an active microbial infection (e.g., pCMV infection) in the transplanted organ, (c) a pCMV infection of human tissue, or any combination thereof. In some cases, the methods may further involve detecting donor-derived cell-free DNA (e.g., non-human derived cell-free DNA, porcine cfDNA, bovine cfDNA, etc.) in order to detect rejection of the transplanted tissue or organ or to rule out rejection. In some cases, the assay for xenotransplant rejection is performed in conjunction with one or more assays for detecting actively replicating microbes, provided herein. In some cases, a quantity of the donor-derived cell-free DNA above a threshold can indicate transplant rejection. In some cases, a relative increase in the quantity of donor-derived cell-free DNA over time can indicate transplant rejection. The present disclosure provides methods that can identify causes of transplant injury. The methods of the disclosure may provide a clearer picture of the role played by an infection, if any, in a transplant injury.

Similarly, the methods provided herein can be useful in the context of allotransplantation. In some embodiments, the method comprises detecting mcfDNA (e.g., CMV mcfDNA) and cfDNA from the transplanted tissue (e.g., donor-derived cfDNA). For example, relatively high concentrations of CMV mcfDNA and donor-derived cfDNA may be observed in a body fluid such as plasma. This can, in some cases, occur after withdrawal of a support or therapy to a recipient of a transplanted tissue. In some embodiments, the methods include determining that elevated microbial cell-free DNA (e.g., CMV mcfDNA) may have been derived from (a) latent infections that were exposed as the transplanted tissue failed, or (b) an active microbial infection (e.g., CMV infection) in the transplant recipient. In some embodiments, the infection is caused by a virus (e.g., cytomegalovirus (CMV)). The methods may further comprise detecting donor-derived cfDNA to identify or rule out transplant rejection. In some cases, the assay for allotransplant rejection is performed in conjunction with one or more assays for detecting actively replicating microbes, provided herein. In some cases, a quantity of the donor-derived cell-free DNA above a threshold can indicate transplant rejection. In some cases, a relative increase in the quantity of donor-derived cell-free DNA over time can indicate transplant rejection. The present disclosure provides methods that can identify causes of transplant injury. The methods of the disclosure may provide a clearer picture of the role played by an infection, if any, in a transplant injury.

In some embodiments, the method involves use of fragment patterns (or fragmentomics) to provide a quantitative proxy for microbial growth rates by bioinformatically comparing coverage at the origin and terminus of replication. In some embodiments, the method involves use of abundance of genetic material at a particular genomic region to provide a quantitative proxy for microbial growth rates by bioinformatically comparing coverage at the origin and terminus of replication. In some examples, an increase in viral (e.g., porcine CMV (pCMV)) or bacterial mcfDNA derived from genomic loci near the origin of replication indicates the presence of active replication. In some cases, the fragment patterns are identified using massively parallel sequencing or Next Generation Sequencing. In some cases, an increase in microbial concentration (e.g., pCMV concentration), non-uniform mcfDNA surrounding the origin of replication, and/or the emergence of shorter mcfDNA fragments over time, in any combination, can indicate the presence of active microbial replication (e.g., active viral replication). Amplification (e.g., qPCR, PCR) can be used to detect active replication by analysis of activity at an origin of replication. Such amplification can be used independently to assess active replication or in conjunction with a fragmentomics approach. This disclosure can, in some cases, concern methods of identification of a fragmentation pattern of mcfDNA and use of the fragmentation pattern as a biomarker, often as a biomarker for an infection. In some cases, the fragmentation pattern of a particular mcfDNA (e.g., CMV) is used to identify active infection of a particular microbe (e.g., CMV) without examination of the ORI or comparison of abundance of reads at the ORI to one or more of the termini.

Often, the methods provided herein focus on the origin of replication of the microbe in order to assess active infection, often in comparison with abundance of fragments around the terminus of the microbial replication. In some cases, absolute levels of mcfDNA (e.g., pCMV mcfDNA) are monitored over time and compared with assays that interrogate genomic loci near the origin of replication or replication terminus. In some cases, an increase in mcfDNA near the origin of replication is a prelude, or indication, of a subsequent increase in absolute mcfDNA (e.g., pCMV mcfDNA), which can appear, in some cases, between 1-18 days (or at least 1, 2, 3, 4, 5, 6, or 7 days, or at most 7, 9, 10, 11, 12, 13, 14, 15, or 18 days) following the observed activity near the origin of replication. In some cases, the dynamics of mcfDNA (e.g., pCMV mcfDNA and pCMV mcfDNA) near the origin of replication over time can together, or individually, provide an indication as to a cause of a xenograft injury, as well as a response to treatment, such as treatment with an antiviral drug (e.g., anti-replication antiviral, ganciclovir, valacyclovir, cidofovir) or a treatment with an antibiotic drug. The dynamics at the origin of replication region can also be analyzed in conjunction with the dynamics at the replication terminus region.

When an actively replicating microbe is detected, in some cases, an antimicrobial treatment (e.g., antiviral, antibiotic) may be administered to a transplant recipient. In some cases, when an actively replicating microbe is detected, the dose of the antimicrobial treatment may need to be adjusted, e.g., the dose may need to be increased. In some cases, active replication may indicate that the existing antimicrobial treatment should be reduced or terminated and that a new or secondary antimicrobial treatment should be administered. Conversely, when active replication is not detected using a method provided herein, an antimicrobial treatment (e.g., antiviral, antibiotic) may be administered to a transplant recipient. In some cases, when an actively replicating microbe is not detected, the dose of the antimicrobial treatment may need to be adjusted, e.g., the dose may need to be decreased. In some cases, an absence of active replication may indicate that the existing antimicrobial treatment is effective, indicating that an existing regimen should be maintained or reduced. In some cases, the methods provided herein comprise detecting mcfDNA strand asymmetry around the viral origin of replication. In some embodiments, such asymmetry may represent the capture of single-stranded DNA intermediates of DNA replication.

In some cases, the methods provided herein involve detecting genomic nucleic acids (e.g., DNA, cfDNA) from multiple sources (e.g., 2, 3, 4, or more genomic sources). For example, in the case of a human recipient of a xenotransplant, the methods provided herein may detect genetic material (e.g., cfDNA or RNA) from a human recipient (e.g., human cfDNA), a xenotransplant (e.g., a pig donor in the case of a porcine xenotransplant), and a microbe (e.g., a virus such as CMV or a virus that is associated with infection of a xenotransplant such as pCMV). In some cases, in the case of a human recipient of an allograft, the method may involve detecting genetic material (e.g., cfDNA or RNA) from the human recipient (e.g., human cfDNA), the allotransplant (e.g., donor cfDNA), and the microbe (e.g., a virus such as CMV or pCMV). In some cases, the microbe may be involved in an active infection of the transplant, in a latent or dormant infection of the transplant, in contamination of the transplant during a surgical procedure. In some cases, the microbe may be associated with an active, dormant, latent or previous infection of the recipient.

In some cases, the methods may involve monitoring different populations of cfDNA in a subject over time. In some cases, the dynamics of human cfDNA, mcfDNA (e.g., pCMV mcfDNA), and transplant cfDNA (e.g., porcine cfDNA, human donor-derived cfDNA) over time can together, or individually, provide an indication as to a cause of a transplant or xenograft injury, as well as a response to treatment, such as treatment with an antiviral (e.g., anti-replication antiviral, ganciclovir, valacyclovir, cidofovir) or antibiotic (e.g., trimethoprim, sulfamethoxazole, cefuroxime). In some cases, the methods may involve monitoring different populations of cfDNA in a subject over time, for example, to detect a response to treatment or a trend in response to treatment.

In some cases, the methods comprise detection of both mcfDNAs (e.g., pCMV mcfDNA) and human cfDNA or recipient cfDNA. In some embodiments, the method involves detection and/or monitoring of a population of ˜55 nt mcfDNA (e.g., pCMV mcfDNA) and/or ˜55 nt human cell-free DNA. In some embodiments, a progressive increase in pCMV and human sequencing reads derived from the 55 nt population through a treatment regimen is an indication of a single biological process governing the progressively shifting distribution of the cfDNA fragment sizes; for example, a host immune response to pCMV-infected human cells. In some cases, the relative fraction of 55 nt fragments is correlated with changes in viral replication activity. In some cases, the method may comprise performing a PCR (qPCR) assay on mcfDNA (e.g., pCMV mcfDNA) using relatively shorter amplicon lengths than of 50-350 bp. In some cases, the amplicon length is 20, 30, 40, 50, 55, 60, 70, 80, 90, or 100 bp. In some cases, the amplicon length is at least 20, 30, 40, 50, or 55 base pairs. In some cases, the amplicon length is no more than 40, 50, 55, 60, 70, 80, or 90 base pairs.

The fragmentation pattern of cfDNA is generally highly structured and non-uniform across the genome. Human cfDNA fragmentation patterns generally reflect the epigenetic state of the cells from which the cfDNA originated. In some cases, the methods provided herein comprise use of cfDNA fragmentomics biomarkers to differentiate the tissue of origin of human cfDNA.

In some embodiments, the methods disclosed herein can be used to assess an alteration in a device (e.g., xenotransplant, prosthetic device) that has been introduced into a patient (e.g., the methods may be used to detect an infection associated with a transplant or an infection introduced into the subject by the transplant, or microbial contamination of a prosthetic device) as well as to assess adverse consequences to the recipient of the transplanted device such as an immune response, inflammation, or rejection (e.g., rejection of a xenotransplant, an allotransplant transplant or an autologous transplant). In some cases, the methods are used to assess infection resulting from contamination during a surgical procedure. A non-limiting example of a change in the functioning of a device includes a development of an infection, a change in an infection (increase, stable, or decrease in replication), an infection derived from the device, a change in an infection derived from the device (increase, stable, or decrease in replication), a rejection of the device, and an unknown illness in a subject following the transplantation of the device. Non-limiting examples of a device include a xenotransplant, an allotransplant, a transplant with a prosthetic device, a transplant with a fully prosthetic device, a transplant with a partially prosthetic device (e.g., a prosthetic device comprising both biological and nonbiological material), a transplant with a non-human tissue (e.g., organ or part thereof), and a transplant with a human tissue (e.g., organ or part thereof).

In some embodiments, the microbial fragmentomics methods provided herein provide novel biomarkers for infectious disease diagnosis and prognosis, for understanding microbe-immune interactions, and for studying the response to therapy. In some embodiments, elevated coverage surrounding the origin of replication relative to the replication terminus, surrounding the origin of replication, diversity of fragment lengths, and strand biases can provide information about replication kinetics, or any combination thereof, which is then correlated with the severity of the disease.

This disclosure provides methods concerning fragmentomics of cell-free nucleic acids (e.g., cell-free DNA (cfDNA)) detected in the body fluid of a recipient of transplanted tissue. The methods disclosed herein can be used to assess the success of a surgical procedure such as a transplant surgery. The success of the surgical procedure can be monitored overtime following completion of the surgical procedure. The methods disclosed herein can be used to assess the status of a medical device such as transplanted tissue or organ.

In some cases, the methods may involve monitoring different populations of cfDNA in a subject over time. In some cases, the dynamics of subject cfDNA, mcfDNA, and transplant tissue cfDNA over time can together, or individually, provide an indication as to a cause of a transplantation injury and/or a response to treatment, such as treatment with an antiviral (e.g., ganciclovir, valacyclovir, cidofovir) or antibiotic (trimethoprim, sulfamethoxazole, cefuroxime).

In some embodiments, the method comprises detecting mcfDNA (e.g., pCMV mcfDNA) and cfDNA from the transplanted tissue (e.g., porcine cfDNA). For example, high concentrations of pCMV mcfDNA and porcine cfDNA may be observed in a body fluid such as plasma. This can, in some cases, occur after withdrawal of a support or therapy to a recipient of a transplanted tissue (e.g., recipient of a porcine heart). Without wishing to be bound by theory, the dominant component of cfDNA can arise from the genomes of apoptotic and necrotic cells (REF). In some cases, the methods may involve determining that elevated porcine cfDNA is likely a reflection of increased cell death of the transplanted tissue. In some embodiments, the methods include determining that elevated pCMV mcfDNA may have been derived from (a) latent infections that were exposed as the transplanted tissue failed, (b) an active pCMV infection in the transplanted organ, (c) a pCMV infection of human tissue, or a combination of the above.

The fragmentation pattern of cfDNA is generally highly structured and non-uniform across the genome. Human cfDNA fragmentation patterns generally reflect the epigenetic state of the cells from which the cfDNA originated. In some cases, the methods provided herein comprise use of cfDNA fragmentomics biomarkers to differentiate the tissue of origin of human cfDNA.

In some embodiments, the microbial fragmentomics methods provided herein provide novel biomarkers for infectious disease diagnosis and prognosis. In some embodiments, the microbial fragmentomics methods provided herein provide for understanding microbe-immune interactions. In some embodiments, the microbial fragmentomics methods provided herein provide for determining the response to therapy. In some embodiments, elevated coverage surrounding the origin of replication, diversity of fragment lengths, strand biases, appearance of certain populations of a known fragment length, or any combination thereof can provide information about replication kinetics, which is then correlated with the severity of the disease.

The specific cause of a detected increase in viral concentration can be poorly understood and there may be several possible explanations for the finding, including rejection and/or infections (e.g., bacterial and/or viral). Increases in viral concentration, however, do not necessarily indicate active replication or to what extent the presence of the virus impacts outcome. The poor understanding for transplant (e.g., xenotransplant and allotransplant) injury demonstrates the need for improved methods for transplant analysis. The present disclosure provides methods that can identify active microbial replication, which may provide a clue to a cause of a transplant injury. The methods of the disclosure may provide a clearer picture of the role played by an infection, if any, in a transplant injury.

In the present disclosure, wherever aspects are described herein with the language “comprising,” otherwise analogous aspects described in terms of “consisting of” and/or “consisting essentially of” are also provided. All definitions herein described whether specifically mentioned or not, should be construed to refer to definitions as used throughout the specification and attached claims.

Numeric ranges are inclusive of the numbers defining the range. The term “about” as used herein generally means plus or minus ten percent (10%) of a value, inclusive of the value, unless otherwise indicated by the context of the usage. For example, “about 100” refers to any number from 90 to 110.

The term “transplant device” may apply to any type of transplant. For example, a transplant device comprising biologic and/or artificial material. Non-limiting examples of material in a transplant device can include a human graft, allograft, autologous graft, human tissue, non-human tissue, or non-human material (e.g., an artificial material and/or xenotransplant material). The term “non-human transplant device” may apply to any non-human transplanted material including, but not limed to, artificial, polymer, non-human biological material, or xenotransplant material. The term “xenotransplant device” may apply to any non-human biological material (e.g., tissue, organ, graft). The non-human biological material may be or be derived from porcine, bovine, equine, non-human primate, caprine, or ovine. Non-limiting examples of non-human primates includes chimpanzees, baboons, Rhesus monkeys, orangutans, macaques, and gorillas. The term “human transplant device” may apply to any human transplanted material. The term “allotransplant device” may apply to any human transplanted material. The term “prosthetic device” or “mechanical device” may apply to devices that are entirely or partially comprised of synthetic, artificial, or non-biological material. Non-limiting examples of material that may be a part of a prosthetic device includes metal, fabric, plastic, or polymer (e.g., polyethylene). Non-limiting examples of prosthetic devices include heart valves, joints, eyes, vein valves (e.g., venous valves), vertebral disks, utricle, urethra, sphincter (e.g., urinary and/or rectal), and valves. The terms “partially prosthetic device” or “bioprosthetic device” may apply to a prosthetic device comprised of biological and non-biological material. A non-limiting example of a partially prosthetic device or bioprosthetic device could be a heart valve. For example, the heart valve may comprise biological material, such as from a pig (porcine) or cow (bovine) connected with non-biological material such as metal, fabric, plastic, or polymer (e.g., polyethylene).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

The term “attach” and its grammatical equivalents may refer to connecting two molecules using any mode of attachment. For example, attaching may refer to connecting two molecules by chemical bonds or other method to generate a new molecule. Attaching an adapter to a nucleic acid may refer to forming a chemical bond between the adapter and the nucleic acid. In some cases, attaching is performed by ligation, e.g., using a ligase. For example, a nucleic acid adapter may be attached to a target nucleic acid by ligation, via forming a phosphodiester bond catalyzed by a ligase. In some cases, an adapter can be attached to a target nucleic acid (or copy thereof) using a primer extension reaction.

As used herein, the term “or” is used to refer to a nonexclusive or, such as “A or B” includes “A but not B,” “B but not A,” and “A and B,” unless otherwise indicated.

As used herein, “a”, “an”, and “the” can include plural referents unless otherwise limited expressly or by context.

### Subjects

The term “subject” as used herein includes patients, particularly human patients. In some embodiments, the subject is an animal (e.g., farm animal or lab animal) or a domestic pet. In some embodiments, the animal can be an insect, a dog, a cat, a horse, a cow, a rodent (e.g., a mouse or a rat), a pig, a fish, a bird, a chicken, a sheep, an ape, or a monkey. The term “subject” also encompasses mammals, veterinary animals, dogs, cats, rodents, farm animals, and primates. The primates may comprise humans. The primates may comprise non-human primates. Non-limiting examples of non-human primates include simians (e.g., monkeys and apes) and prosimians (e.g., lemurs).

In some embodiments, the subject is a transplant recipient. In some embodiments, the subject is a xenotransplant recipient. In some embodiments, the subject is an allotransplant recipient. In some embodiments, the subject is a recipient of a prosthetic device. In some embodiments, the subject is a recipient of a graft. In some embodiments the subject is a recipient of an organ, tissue or graft transplant. In some embodiments, the subject is a recipient of a xenograft. In some embodiments the subject is a recipient of a xenotransplant of an organ. In some embodiments, the subject is a recipient of a transplant from a different species than the subject. For example, the subject can be a human and a recipient of a non-human organ or graft, such as a non-human mammalian organ or graft. In some embodiments, the subject is a recipient of a transplant from a pig. For example, the subject can be a human and a recipient of a porcine organ or graft. In some embodiments, the subject is a recipient of a transplant from a cow. For example, the subject can be a human and a recipient of a bovine organ or graft. Non-limiting examples of transplant organs include a heart, a lung or lungs, a kidney, a liver, and a pancreas. The heart, lung or lungs, kidney, liver, or pancreas may be from pig. The heart, lung or lungs, kidney, liver, or pancreas may be from cow. The heart, lung or lungs, kidney, liver, or pancreas may be from a non-human mammal.

In some embodiments, the subject is a child. In some embodiments, a child is less than about 18 years of age. In some embodiments, the subject is a pediatric patient. In some embodiments, a subject is an adult. In some embodiments, a subject is less than about 25 years of age. In some embodiments, a subject is elderly. In some embodiments a subject is more than 65 years of age. In some cases, the subject has a high risk of experiencing a viral, bacterial, or fungal infection.

An initial sample can be derived from any subject (e.g., a human subject, a non-human subject, etc.). The subject can be healthy. In some embodiments, the subject is a human patient having, suspected of having, or at risk of having, a disease or infection. In some embodiments, the disease or infection is pathogen-related. In some embodiments, the disease or infection is related to a microbe. In some embodiments, the microbe is a bacterium. In some embodiments, the microbe is a virus. In some embodiments, the microbe is a fungus. In some embodiments, the microbe is associated with a transplant. In some embodiments, the microbe is associated with a transplanted organ. In some embodiments, the microbe is associated with a transplanted graft. In some embodiments, the microbe is associated with a xenotransplant. In some embodiments, the microbe is associated with a xenotransplanted organ. In some embodiments, the microbe is associated with a xenograft. In some embodiments, the microbe is associated with an allotransplant. In some embodiments, the microbe is associated with an allotransplanted organ. In some embodiments, the microbe is associated with an allograft.

A human subject can be a male or female. In some embodiments, the sample can be from a human embryo or a human fetus. In some embodiments, the human can be an infant, child, teenager, adult, or elderly person. In some embodiments, the subject is a female subject who is pregnant, suspected of being pregnant, or planning to become pregnant.

In some embodiments, the subject is a human subject who has undergone an organ transplant or who is planning to undergo organ transplant. In some embodiments, the subject is a human subject who has undergone a transplant with a transplant device or who is planning to undergo a transplant with a transplant device. In some embodiments, the subject is a human subject who has undergone a transplant with a non-human device or who is planning to undergo a transplant with a non-human device. In some embodiments, the subject is a human subject who has undergone a xenotransplant or who is planning to undergo a xenotransplant. In some embodiments, the subject is a human subject who has undergone a transplant with a xenotransplant device or who is planning to undergo a transplant with a xenotransplant device. In some embodiments, the subject is a human subject who has undergone a transplant with a human device or who is planning to undergo a transplant with a human device. In some embodiments, the subject is a human subject who has undergone an allotransplant or who is planning to undergo an allotransplant. In some embodiments, the subject is a human subject who has undergone a transplant with an allotransplant device or who is planning to undergo a transplant with an allotransplant device. In some embodiments, the subject is a human subject who has undergone a transplant with a prosthetic device or who is planning to undergo a transplant with a prosthetic device. In some embodiments, the subject is a human subject who has undergone a transplant with a partially prosthetic device or who is planning to undergo a transplant with a partially prosthetic device. In some embodiments, the subject is a human subject who has undergone a transplant with a bioprosthetic device or who is planning to undergo a transplant with a bioprosthetic device.

In some cases, risk factors for contracting an infection or for progression to an invasive disease are genetic variants in the subject's genomic DNA. Genetic variants that can be risk factors for infection include but are not limited to, single-nucleotide polymorphisms, deletions, insertions, or the like. In some other cases, subjects can have family history of disease such as gastric cancer, family history of lymphocytic gastritis, hyperplastic gastric polyps, or hyperemesis gravidarum.

The subject may have or be at risk of having another disease or co-infection by more than one pathogen. In some cases, the subject is immunosuppressed (e.g., organ transplant patients, xenotransplant patients, allotransplant patients). In some embodiments, the subject may have taken or is taking an immunosuppressant drug. Non-limiting examples of immunosuppressant drugs include tacrolimus, anti-CD3, prednisone, cyclosporine, azathioprine, mycophenolate mofetil, sirolimus, everolimus, and alemtuzumab.

In some embodiments disclosed herein, a subject is at risk of having an infection (e.g., high risk of having an infection), particularly at the time of collecting a sample from the subject. As used herein, a subject with a “high risk” of experiencing an infection is a subject with a risk that is higher than that of a healthy subject. For example, a patient who is immunocompromised is generally at high risk of experiencing an infection when compared to a healthy patient who is not immunocompromised. In some embodiments, the subject has, is suspected of having, or is at risk (e.g., high risk) of having an infection by a bacterium, a fungus, a virus, a parasite, or any combination thereof, or symptoms of such infection. In some embodiments, the infection is a viral infection (e.g., localized or systemic infection). In some embodiments, the infection is a fungal infection (e.g., invasive fungal infection)). In some embodiments, the infection is a bacterial infection (e.g., localized infection). In some embodiments, the bacterial infection is a gram-negative bacterial infection. In some embodiments, the bacterial infection is a gram-positive bacterial infection. In some embodiments, the viral, bacterial, or fungal infection is susceptible to empirical antimicrobial therapy. In some embodiments, the viral, bacterial, or fungal infection is not susceptible to empirical antimicrobial therapy. In some embodiments, an antibiotic is used against the bacterial infection. Non-limiting examples of antibiotics include amoxicillin, cefaclor, cefradine, ceftriaxone, cefuroxime, cefalexin, cefixime, clindamycin, co-amoxiclav, doxycycline, flucloxacillin, metronidazole, minocycline, nitrofurantoin, phenoxymethylpenicillin, ethambutol, isoniazid, and pyrazinamide. In some embodiments, an antifungal is used against the fungal infection. Non-limiting examples of antifungals include nystatin, fluconazole, clotrimazole, triazole, voriconazole, dapsone, pentamidine, sulfamethoxazole/trimethoprim, and posaconazole. In some embodiments, an antiviral is used against the viral infection. Non-limiting examples of antivirals include oseltamivir, zanamivir, valacyclovir, letermovir, valganciclovir, and lamivudine. In some embodiments, the subject is diagnosed with having an infection or predicted to be at risk of an infection using methods disclosed herein. In some embodiments, a subject is predicted to be at risk of having an infection or at risk of developing symptoms of infection using methods disclosed herein.

In some cases, the subject may present with one or more clinical symptoms. Non-limiting examples of clinical symptoms can include aching or burning pain in the abdomen, abdominal pain that worsens when the stomach is empty, nausea, loss of appetite, frequent burping, bloating in the stomach area, weight loss, severe or persistent abdominal pain, difficulty swallowing, bloody or black tarry stools, and/or bloody or black vomit. Additional clinical symptoms are known in the art.

The subject may be infected by a pathogen or microorganism of any type, including bacterial, viral, fungal, parasitic, prokaryotic, eukaryotic, etc. In some embodiments, the pathogen is associated with a transplant procedure. In some embodiments, the pathogen is a common infection of a transplant recipient. In some cases, the pathogen is known. In some cases, the pathogen may be a known commensal. In some cases, the pathogen is not known. In some cases, the subject may have an active or latent infection. In some cases, the subject is infected, but the infection is below the level of diagnostic sensitivity of other tests previously conducted on the subject. In some cases, the subject is infected but asymptomatic or the infection is at a sub-clinical level. In some embodiments, the subject is infected, but the infection is dormant. In some embodiments, the subject is infected by a virus. In some embodiments, the subject was infected by a virus. In some embodiments, a virus becomes a non-replicating virus. In some embodiments, the virus becomes a damaged virus.

The subject may have a disease, disorder, or condition. The methods disclosed herein may identify a disease, disorder, or condition of the subject. The disease, disorder, or condition of the subject may be a xenotransplant injury. The disease, disorder, or condition of the subject may be an allotransplant injury. The disease, disorder, or condition of the subject may be a transplant injury. The disease, disorder, or condition of the subject may be a transplant rejection. The disease, disorder, or condition of the subject may be an active microbial infection. The disease, disorder, or condition of the subject may be a latent microbial infection. The disease, disorder, or condition of the subject may be an active bacterial infection. The disease, disorder, or condition of the subject may be a latent bacterial infection. The disease, disorder, or condition of the subject may be an active viral infection. The disease, disorder, or condition of the subject may be a latent viral infection. The disease, disorder, or condition of the subject may be a prosthetic injury. The prosthetic injury may be an infection. The infection causing the prosthetic injury may be bacterial.

In some embodiments, the methods disclosed herein identify active replication of a microbe. The microbe may be present in a biological sample. The biological sample may be from a subject, such as a patient. The subject may be known to have the microbe present. The subject may be suspected of having the microbe present.

### Sample Type

In some embodiments, a sample is collected from a subject (e.g., a patient). In some embodiments, the sample is a biological sample. In some embodiments, the biological sample is a biological fluid sample. The samples analyzed in the methods provided herein can be any type of clinical sample. In some cases, the samples contain cells, tissue, or a bodily fluid. In some embodiments, the biological sample is a cell-free biological sample. In some embodiments, the biological sample is a cell-free biological fluid sample. In some embodiments, the sample is a liquid or fluid sample. In some cases, the sample is a bodily fluid. In some cases, the sample is whole blood, blood, plasma, serum, urine, stool, saliva, lymph, spinal fluid, synovial fluid, bronchoalveolar lavage, nasal swab, respiratory secretions, vaginal fluid, amniotic fluid, semen, cerebrospinal fluid, or menses. In some cases, the biological sample is a plasma sample. In some cases, the biological fluid sample is a plasma sample. In some cases, the sample is made up of, in whole or in part, cells or tissue. In some cases, cells, cell fragments, or exosomes are removed from the sample, such as by centrifugation or filtration.

In some embodiments, the biological sample comprises nucleic acids from one or more sources. Non-limiting examples of the one or more sources includes subject nucleic acids, microbial nucleic acids, and donor nucleic acids. For example, a biological sample from a human subject that was the recipient of a bovine liver and exhibiting signs of infection can comprise nucleic acids that are from the human subject, the bovine donor, and the microbe causing the infection. As another example, a biological sample taken from a human subject that was the recipient of a fully prosthetic device and exhibiting signs of an infection can comprise nucleic acids that are from the human subject and the microbe causing the infection. In some embodiments, the microbial nucleic acids comprise fragments of microbial nucleic acids. In some embodiments, the fragments of microbial nucleic acids are of different lengths.

In some embodiments, a biological sample is a whole blood sample. In some embodiments, the sample is a cell-free sample, such as a plasma sample or a cell-free plasma sample. In some embodiments, the sample is a sample of isolated or extracted nucleic acids (e.g., DNA, RNA, cell-free DNA). In some embodiments, the sample is a sample comprising cell-free nucleic acids. In some embodiments, the plasma sample is collected by collecting blood through venipuncture. In some embodiments, a specimen is mixed with an additive immediately after collection. In some cases, the additive is an anti-coagulant. In some cases, the additive prevents degradation of nucleic acids. In some cases, the additive is EDTA. In some embodiments, measures can be taken to avoid hemolysis or lipemia. In some embodiments, a sample is processed or unprocessed. In some embodiments, a sample is processed by extracting nucleic acids from a biological sample. In some embodiments, DNA is extracted from a sample. In some embodiments, nucleic acids are not extracted from the sample. In some embodiments, the sample comprises cell-free nucleic acids and the nucleic acids are not extracted from the sample. In some embodiments, the cell-free nucleic acids are cell-free DNA or cell-free RNA. In some embodiments, a sample comprises nucleic acids. In some embodiments, a sample consists essentially of nucleic acids.

In some cases, the methods provided herein comprise processing whole blood into a plasma sample or a serum sample. The sample can be any fraction of a processed whole blood sample. In some embodiments, such processing comprises centrifuging the whole blood in order to separate the plasma from blood cells. In some embodiments, such processing comprises centrifuging the whole blood in order to separate the serum from blood cells. In some cases, the methods provided herein further comprises methods, such as for example, a second centrifugation, ultracentrifugation, filtration, ultrafiltration, or gel separation. In some cases, the second centrifugation, filtration, ultrafiltration, or gel separation may be performed to remove bacterial cells, viral cells (e.g., intact viruses, partial viruses, viral capsids), intact microbes, cellular debris, or any combination thereof. In some cases, the second centrifugation is performed at a higher speed. In some cases, the second centrifugation is performed at a lower speed. In some cases, the second centrifugation is at a relative centrifugal force (rcf) of at least about 1,000 rcf, at least about 1,500 rcf, at least about 2,000 rcf, at least about 3,000 rcf, at least about 4,000 rcf, at least about 5,000 rcf, at least about 6,000 rcf, at least about 8,000 rcf, at least about 10,000 rcf, at least about 12,000 rcf, at least about 14,000 rcf, at least about 16,000 rcf, at least about 20,000 rcf, at least about 40,000 rcf, at least about 60,000 rcf, at least about 80,000 rcf, or at least about 100,000 rcf. In some embodiments, multiple centrifugation steps are performed to remove bacterial cells, viral cells (e.g., intact viruses, partial viruses, viral capsids), intact microbes, cellular debris, or any combination thereof. The multiple centrifugation steps may be carried out at different centrifugal speeds from one another. In some embodiments, the supernatant is removed following centrifugation.

In some cases, the method comprises collecting, obtaining, or providing a sample. In some cases, the method comprises collecting, obtaining, or providing multiple samples, e.g., multiple samples from the subject or patient. In some embodiments, the sample is collected when the subject has an infection. In some cases, the sample is collected prior to the subject having an infection. In some cases, the sample is collected while the subject is receiving treatment for an infection. In some cases, the sample is collected after the subject has received a treatment for an infection. In some embodiments, the sample is collected when the subject has an infection. In some cases, the sample is collected before the subject undergoes a tissue transplant. In some cases, the sample is collected after the subject has undergone a tissue transplant. In some cases, the sample is collected before the subject undergoes a transplant with a transplant device. In some cases, the sample is collected after the subject has undergone a transplant with a transplant device. In some cases, the sample is collected before the subject undergoes a xenotransplant. In some cases, the sample is collected after the subject has undergone a xenotransplant. In some cases, the sample is collected before the subject undergoes an allotransplant. In some cases, the sample is collected after the subject has undergone an allotransplant. In some cases, the sample is collected before the subject undergoes a transplant with a prosthetic device. In some cases, the sample is collected after the subject has undergone a transplant with a prosthetic device. In some cases, the sample is collected before the subject undergoes a transplant with a partially prosthetic device. In some cases, the sample is collected after the subject has undergone a transplant with a partially prosthetic device. In some cases, the sample is collected before the subject undergoes a transplant with a bioprosthetic device. In some cases, the sample is collected after the subject has undergone a transplant with a bioprosthetic device. In some cases, additional samples are collected from the subject over time. In some embodiments, a second sample is collected from the subject at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days after the collection of an initial (or other) sample from the subject

In some embodiments, a plurality of samples is collected over a series of time points. In some embodiments, a plurality of samples is collected to monitor an onset of a disease, to monitor progression of a disease, to detect a response to treatment for the disease, to monitor rejection of a tissue transplant, or any combination thereof. In some embodiments, the plurality of samples is at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected before onset of a symptom and/or before a tissue transplant. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected after onset of a symptom and/or after a tissue transplant. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected before and after onset of a symptom and/or before a tissue transplant. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected over a period of time. In some embodiments, a plurality of samples is collected on consecutive days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected on consecutive days. In some embodiments, a plurality of samples is collected on alternate days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples can be collected on alternate days. In some embodiments, a plurality of samples is collected about every 4 days. In some embodiments, a plurality of samples is collected about every 5 days. In some embodiments, a plurality of samples is collected about every 6 days. In some embodiments, a plurality of samples is collected about every 7 days. In some embodiments, a plurality of samples is collected about every 8 days. In some embodiments, a plurality of samples is collected about every 9 days. In some embodiments, a plurality of samples is collected about every 10 days. In some embodiments, the collection of samples can be interspersed between days when no sample is collected. In some embodiments, a schedule of sample collection can repeat over several days. In some embodiments, a schedule of sample collection can repeat over 2 days, over 3 days, over 4 days, over 5 days, over 6 days, over 7 days, over 8 days, over 9 days, over 10 days, over 11 days, over 12 days, over 13 days, over 14 days, over 15 days, over 16 days, over 17 days, over 18 days, over 19 days, over 20 days, over 21 days, or over 22 days. In some embodiments, a schedule of sample collection can repeat on the same day, collecting multiple samples from a subject throughout the 24 hours.

A sample disclosed herein can comprise a target nucleic acid (e.g., target DNA, target RNA). In some embodiments, the target nucleic acid is a cell-free nucleic acid. In some embodiments, a target nucleic acid is a microbial cell-free nucleic acid (mcfNA). In some embodiments, the mcfNA comprises microbial RNA. In some embodiments, the mcfNA comprises microbial DNA. In some embodiments, a target nucleic acid is a cell-free nucleic acid. For example, the sample can comprise microbial cell-free nucleic acids (e.g., mcfDNA) that comprises a microbial target DNA (e.g., mcfDNA derived from a microbe, which can include pathogenic microbes). Exemplary microbes that can be detected by the methods provided herein include bacteria, fungi, parasites, and viruses. In some embodiments, the sample can comprise cell-free nucleic acids derived from the transplanted tissue. For example, if a human subject receives a heart transplant wherein the heart is porcine, the cell-free nucleic acids may comprise porcine cell-free nucleic acids. In some embodiments, a cell-free nucleic acid is a circulating cell-free nucleic acid. In some embodiments, a cell free nucleic acid can comprise cell-free DNA. In some embodiments, a cell free nucleic acid can comprise cell-free RNA.

In some cases, a target nucleic acid (e.g., target cfmDNA) may make up only a very small portion of a sample, e.g., less than 0.1%, less than 0.01%, less than 0.001%, less than 0.0001%, less than 0.00001%, less than 0.000001%, less than 0.0000001% of the total nucleic acids (e.g., cfDNA) in a sample. In some cases, a concentration of cell-free nucleic acids (e.g., DNA, mRNA, RNA) may be in a range of 0.01-10,000 ng/ml, e.g., (about 0.01, 0.1, 1, 5, 10, 20, 30, 40, 50, 80, 100, 1000, 5000, 10000 ng/ml). In some cases, the total concentration of cell-free nucleic acids in a sample is outside of this range (e.g., less than 0.01 ng/ml; in other cases, the total concentration is greater than 10,000 ng/ml). This may be the case with cell-free nucleic acid (e.g., DNA) samples that are predominantly made up of human DNA and/or RNA. In such samples, pathogen target nucleic acids may have scant presence compared to the human or host nucleic acids.

In some embodiments, a length of a nucleic acid can vary. In some embodiments, a nucleic acid or nucleic acid fragment (e.g., dsDNA fragment, RNA, or randomly sized cDNA) can be less than 1000 bp, less than 800 bp, less than 700 bp, less than 600 bp, less than 500 bp, less than 400 bp, less than 300 bp, less than 200 bp, or less than 100 bp. In some embodiments, a nucleic acid or nucleic acid fragment (e.g., dsDNA fragment, RNA, or randomly sized cDNA) can be at least 10 bp, at least 20 bp, at least 30 bp, at least 40 bp, at least 50 bp, at least 55 bp at least 60 bp, at least 70 bp, at least 80 bp, or at least 100 bp. In some embodiments, a DNA fragment can be about 40 to about 100 bp, about 50 to about 125 bp, about 100 to about 200 bp, about 150 to about 400 bp, about 300 to about 500 bp, about 100 to about 500 bp, about 400 to about 700 bp, about 500 to about 800 bp, about 700 to about 900 bp, about 800 to about 1000 bp, or about 100 to about 1000 bp. In some embodiments, a nucleic acid or nucleic acid fragment (e.g., dsDNA fragment, RNA, or randomly sized cDNA) can be within a range from about 30 to about 300 bp, such as within a range from about 50 to about 280 bp. In some cases, the nucleic acid fragments comprise similar nt lengths as those provided here for bp.

### Transplant

The methods disclosed herein can comprise transplanting, e.g., transplanting tissue, a graft, an organ, a medical device into a recipient. Transplanting can be autotransplanting, allotransplanting, xenotransplanting, or any other transplanting. For example, transplanting can be xenotransplanting. Transplanting can also be allotransplanting. The methods may also comprise detection of nucleic acids within a sample from a transplant recipient.

The terms “implant” or “transplant” or “graft” as used herein can be interchangeable and shall be understood to refer to the act of inserting tissue or an organ into a subject under conditions that allow the tissue or organ to become vascularized; and shall also refer to the so-inserted (e.g., “implanted” or “transplanted” or “grafted”) tissue or organ. Conditions favoring vascularization of a graft in a mammal can comprise a localized tissue bed at the site of the graft having an extensive blood supply network. Non-limiting examples of transplants can include bone marrow, heart, kidney, liver, and pancreas. Other examples of transplants include heart valves, vascular grafts, skin grafts, dura mater grafts, pericardium grafts, cartilage grafts and implants.

Transplanting can include transplantation of a tissue such as an organ. In exemplary embodiments, the organ is a kidney, liver, lung, heart, pancreas or other solid organs. Examples of tissues contemplated herein include, without limitation, epithelial and connective tissues. Transplants involving more than one organ or organ fragment are also contemplated herein. For example, transplants involving a lung (or lung fragment) and heart (or fragment thereof) are contemplated herein.

In some embodiments, the transplant or implant is a prosthetic device. The prosthetic device may be fully prosthetic or partially prosthetic. In some embodiments, the prosthetic device is a prosthetic valve. In some embodiments, the transplant includes a prosthetic valve. In some embodiments, the prosthetic valve is a prosthetic heart valve. In some embodiments, the prosthetic valve is mechanical. In some embodiments, the mechanical prosthetic valve is fully prosthetic. A fully prosthetic device may be made entirely of non-biological materials. Non-limiting examples of mechanical prosthetic valves include a caged ball valve, caged disk valve, tilting disk valve, single leaflet valve, and a bi-leaflet valve. In some embodiments, the prosthetic valve is bioprosthetic. In some embodiments, the bioprosthetic valve is partially prosthetic. In some embodiments, the bioprosthetic valve comprises a biologic and synthetic material. The synthetic material may be non-biologic materials. In some embodiments, the bioprosthetic valve comprises a biologic. In some embodiments, the biologic of the bioprosthetic valve is non-human. Non-limiting examples of non-human biologics in bioprosthetic valves include pigs (porcine), cows (bovine), horse (equine), goat (Caprine), and non-human primates (e.g., baboon or chimpanzee). In some embodiments, the synthetic material of a bioprosthetic valve includes one or more of metal, fabric, plastic, or polymer. For example, a prosthetic device may be a prosthetic heart valve made with porcine tissue and one or more synthetic materials. In some embodiments, the prosthetic device includes one or more of metal, titanium, cobalt, pyrolytic carbon, or a polymer. In some embodiments, the synthetic material of the prosthetic device includes one or more of metal, titanium, cobalt, pyrolytic carbon, or a polymer. In some embodiments, the synthetic material of the partially prosthetic device includes one or more of metal, titanium, cobalt, pyrolytic carbon, or a polymer. In some embodiments, the synthetic material of the fully prosthetic device includes one or more of metal, titanium, cobalt, pyrolytic carbon, or a polymer. In some embodiments, the synthetic material of a bioprosthetic valve is used to form a stent. In some embodiments, the synthetic material of a bioprosthetic valve is used to form a scaffold. In some embodiments, the synthetic material of a bioprosthetic valve is used to attach the non-human biologic components of the bioprosthetic valve to a synthetic material of the bioprosthetic valve. In some embodiments, the synthetic material of a bioprosthetic valve is used to connect non-human biologic components of the bioprosthetic valve.

“Xenotransplantation” and its grammatical equivalents as used herein can encompass any procedure that involves transplantation, implantation, or infusion of cells, tissues, or organs into a recipient, where the recipient and donor are different species. Transplantation of the cells, organs, and/or tissues described herein can be used for xenotransplantation in into humans. Xenotransplantation includes but is not limited to vascularized xenotransplant, partially vascularized xenotransplant, unvascularized xenotransplant, xenodressings, xenobandages, and xenostructures.

“Allotransplantation” and its grammatical equivalents as used herein can encompasses any procedure that involves transplantation, implantation, or infusion of cells, tissues, or organs into a recipient, where the recipient and donor are the same species. Transplantation of the cells, organs, and/or tissues described herein can be used for allotransplantation in into humans. Allotransplantation includes but is not limited to vascularized allotransplant, partially vascularized allotransplant, unvascularized allotransplant, allodressings, allobandages, and allostructures.

In some cases, the methods provided herein are performed in conjunction with another assay in order to provide additional information regarding the status of a transplanted organ, graft, or medical device. As such, the methods may further comprise detecting absolute quantities of mcfNA (e.g., mcfDNA, mcfRNA), absolute quantities of host DNA, or absolute quantities of donor-derived cfNA, in any combination thereof. The presence of donor-derived cfNA above a certain threshold may indicate rejection of the transplanted tissue or organ. A progressive increase of donor-derived cfNA over time may indicate continued rejection of the transplanted tissue or organ. A decrease of donor-derived cfNA may indicate reduced rejection of the transplanted tissue or organ. A decrease of donor-derived cfNA may indicate no rejection of the transplanted tissue or organ taking place. A decrease of donor-derived cfNA may indicate improvement in the rejection of the transplanted tissue or organ. Little to no donor-derived cfNA may indicate no rejection of the transplanted tissue or organ taking place. In some cases, the ddcfNA is detected using polymorphisms unique to the donor compared to the host. For example, the ddcfNA may be detected by detected single nucleotide polymorphisms or SNPs.

### Sequencing

In some embodiments, nucleic acids (e.g., cell-free nucleic acids) are extracted from a sample. In some embodiments, the cell-free nucleic acids are not extracted prior to library preparation. In some embodiments, the cell-free nucleic acids derived from the sample are cell-free prior to extraction. In some embodiments, isolated nucleic acids (e.g., extracted DNA, extracted RNA) can be used to prepare DNA libraries. In some embodiments, DNA libraries can be prepared by attaching adapters to nucleic acids. In some embodiments, adapters can be used for sequencing of nucleic acids. In some embodiments, nucleic acids can comprise DNA. In some embodiments, nucleic acids containing adapters can be sequenced to obtain sequence reads. In some embodiments, a sample (e.g., a plasma sample comprising mcfDNA) is mixed with adapters prior to extracting nucleic acids or DNA from the sample. In some embodiments, nucleic acids extracted from a sample (e.g., a plasma sample comprising mcfDNA) are attached to adapters following extraction. In some embodiments, the method further comprises sequencing the cell-free nucleic acids (cfNA) from the biological sample. Sequencing the cfNA from the biological sample may sequence microbial cfNA (mcfNA) in the biological sample. In some embodiments, sequence reads can be produced through high-throughput sequencing (HTS). In some embodiments, sequence reads are produced through massively parallel sequencing. The massively parallel sequencing may be whole genome sequencing. The massively parallel sequencing may be next generation sequencing (NGS). The NGS may be next next generation sequencing. The sequencing may produce sequencing reads. The sequencing reads may be of cfNA from the subject. The sequencing reads may be of cfNA from a microbe in the biological sample of the subject. The sequencing reads may be donor-derived cfNA in the biological sample of the subject. The sequencing reads may be cfNA from a subject in the biological sample of the subject. In some embodiments, HTS can comprise next-generation sequencing (NGS). In some embodiments, sequence reads can be aligned to sequences in a reference dataset. In some embodiments, sequences can be a bacterial sequence aligned to a reference dataset to obtain an aligned sequence read. In some embodiments, a sequence can be a fungal sequence aligned to a reference dataset to obtain an aligned sequence read. In some embodiments, an aligned bacterial sequence, a fungal sequence, or a combination thereof, can be quantified for bacterial sequences or fungal sequences based on aligned sequence reads obtained. In some embodiments, sequences can be a viral sequence aligned to a reference dataset to obtain an aligned sequence read. In some embodiments, an aligned viral sequence can be quantified for viral sequences based on aligned sequence reads obtained. In some embodiments, sequences can be a non-human mammal sequence aligned to a reference dataset to obtain an aligned sequence read. In some embodiments, an aligned non-human mammal sequence can be quantified for non-human mammal sequences based on aligned sequence reads obtained. Non-limiting examples of non-human mammals include pigs (porcine), cows (bovine), horse (equine), goat (Caprine), and non-human primates (e.g., baboon).

In the methods provided herein, nucleic acids can be isolated. In some embodiments, nucleic acids can be extracted using a liquid extraction. In some embodiments, a liquid extraction can comprise a phenol-chloroform extraction. In some embodiments, a phenol-chloroform extraction can comprise use of TRIZOL™, DNAZOL™, or any combination thereof. In some embodiments, nucleic acids can be extracted using centrifugation through selective filters in a column. In some embodiments, nucleic acids can be concentrated or precipitated by known methods, including, by way of example only, centrifugation. In some embodiments, nucleic acids can be bound to a selective membrane (e.g., silica) for the purposes of purification. In some embodiments, nucleic acids can be extracted using commercially available kits (e.g., QIAamp CIRCULATING NUCLEIC ACID KIT™, Qiagen DNeasy KIT™, QIAamp KIT™, Qiagen Midi KIT™, QIAprep SPIN KIT™, or any combination thereof). Nucleic acids can also be enriched for fragments of a desired length, e.g., fragments which are less than 1000, 500, 400, 300, 200 or 100 base pairs in length. In some embodiments, enrichment based on size can be performed using, e.g., PEG-induced precipitation, an electrophoretic gel or chromatography material (Huber et al. (1993) Nucleic Acids Res. 21:1061-6), gel filtration chromatography, or TSK gel (Kato et al. (1984) J. Biochem, 95:83-86), which publications are hereby incorporated by reference in their entireties for all purposes.

In some embodiments, a nucleic acid sample can be enriched for a target nucleic acid. In some embodiments, a target nucleic acid is a microbial cell-free nucleic acid. In some embodiments, a target nucleic acid is a transplant tissue cell-free nucleic acid.

In some embodiments, target (e.g., pathogen, microbial, transplant tissue) nucleic acids are enriched relative to background (e.g., subject) nucleic acids in a sample, for example, by electrophoresis, gel electrophoresis, pull-down (e.g., preferentially pulling down target nucleic acids in a pull-down assay by hybridizing them to complementary oligonucleotides conjugated to a label such as a biotin tag and using, for example, avidin or streptavidin attached to a solid support), targeted PCR, or other methods. Examples of enrichment techniques include but are not limited to: (a) self-hybridization techniques in which a major population in a sample of nucleic acids self-hybridizes more rapidly than a minor population in a sample; (b) depletion of nucleosome-associated DNA from free DNA; (c) removing and/or isolating DNA of specific length intervals; (d) exosome depletion or enrichment; and (e) strategic capture of regions of interest.

In some embodiments, an enriching step can comprise preferentially removing nucleic acids from a sample that are above about 120, about 150, about 200, 250, 280, or about 300 bases in length. In some embodiments, an enriching step comprises preferentially enriching nucleic acids from a sample that are between about 10 bases and about 60 bases in length, between about 10 bases and about 120 bases in length, between about 10 bases and about 150 bases in length, between about 10 bases and about 300 bases in length between about 30 bases and about 60 bases in length, between about 30 bases and about 120 bases in length, between about 30 bases and about 150 bases in length, between about 30 bases and about 200 bases in length, or between about 30 bases and about 300 bases in length. In some embodiments, an enriching step comprises preferentially digesting nucleic acids derived from the host (e.g., subject). In some embodiments, an enriching step comprises preferentially replicating the non-host nucleic acids.

In some embodiments, a nucleic acid library is prepared. In some embodiments, a double-stranded DNA library, a single-stranded DNA library or an RNA library is prepared. A method of preparing a dsDNA library can comprise ligating an adaptor sequence onto one or both ends of a dsDNA fragment. In some cases, the adaptor sequence comprises a primer docking sequence. In some cases, the method further comprises hybridizing a primer to the primer docking sequence and initiating amplification or sequencing of the nucleic acid attached to the adaptor. In some embodiments, the primer or the primer docking sequence comprises at least a portion of an adaptor sequence that couples to a next-generation sequencing platform. In some embodiments, a method can further comprise extension of a hybridized primer to create a duplex, wherein a duplex comprises an original ssDNA fragment and an extended primer strand. In some embodiments, an extended primer strand can be separated from an original ssDNA fragment. In some embodiments, an extended primer strand can be collected, wherein an extended primer strand is a member of an ssDNA library.

In some cases, the library is prepared in an unbiased manner. For example, in some cases, the library is prepared without using a primer that specifically hybridizes to a microbial nucleic acid based on a predetermined sequence of the microbe. For example, in some embodiments, the only amplification performed on the sample involves the use of a primer specific for a sequence of one or more adapters attached to nucleic acids within the sample. In some cases, whole genome amplification is used to prepare the library prior to attachment of the adapters. In some cases, whole genome amplification is not used to prepare the library. In some cases, one or more primers that specifically hybridize to a microbial nucleic acid (e.g., pathogen, viral, fungal, bacterial or parasite nucleic acid) are used to amplify the sample.

In some cases, multiple DNA libraries from different samples (e.g., samples from different patients or subjects) are combined and then subjected to a next generation sequencing assay. In some cases, the libraries are indexed prior to combining in order to track which library corresponds to which sample. Indexing can involve the inclusion of a specific code or bar code in an adapter, e.g., an adapter that is attached to the nucleic acids are to be analyzed. In some cases, the samples comprise a negative control sample or a positive control sample, or both a negative control sample and a positive control sample.

In some embodiments, an end of a dsDNA fragment can be polished (e.g., blunt-ended) or be subject to end-repair to create a blunt end. In some embodiments, an end of a DNA fragment can be polished by treatment with a polymerase. In some embodiments, a polishing can involve removal of a 3′ overhang, a fill-in of a 5′ overhang, or a combination thereof. In some embodiments, a polymerase can be a proof-reading polymerase (e.g., comprising 3′ to 5′ exonuclease activity). In some embodiments, a proofreading polymerase can be, e.g., a T4 DNA polymerase, Pol 1 Klenow fragment, or Pfu polymerase. In some embodiments, a polishing can comprise removal of damaged nucleotides (e.g., abasic sites).

In some embodiments, a ligation of an adaptor to a 3′ end of a nucleic acid fragment can comprise formation of a bond between a 3′ OH group of the fragment and a 5′ phosphate of the adaptor. Therefore, removal of 5′ phosphates from nucleic acid fragments can minimize aberrant ligation of two library members. Accordingly, in some embodiments, 5′ phosphates are removed from nucleic acid fragments. In some embodiments, 5′ phosphates are removed from at least 50%, 55%, 60%, 65%, 70%, 75%, 80%, 85%, 90%, 95%, or greater than 95% of nucleic acid fragments in a sample. In some embodiments, substantially all 5′ phosphate groups are removed from nucleic acid fragments. In some embodiments, substantially all 5′ phosphates are removed from at least 50%, 55%, 60%, 65%, 70%, 75%, 80%, 85%, 90%, 95%, or greater than 95% of nucleic acid fragments in a sample. In some cases, removal of phosphate groups can comprise treating the sample with a phosphatase or a heat-labile phosphatase. In some embodiments, 5′ phosphate groups are not removed from the nucleic acid sample. In some embodiments, attachment or ligation of an adaptor to the 5′ end of the nucleic acid fragment is performed.

What follows are non-limiting examples of methods provided by this disclosure. In some cases, plasma is spiked with a known concentration of synthetic normalization molecule controls. In some cases, the plasma is then subjected to cell-free NA (cfNA) extraction (e.g., extraction of cell-free DNA). The extracted cfNA can be processed by end-repair and ligated to adapters containing specific indexes to end-repaired cfDNA. The products of the ligation can be purified by beads. In some embodiments, the cfDNA ligated to adapters can be amplified with P5 and P7 primers, and the amplified, adapted cfDNA is purified.

Purified cfDNA attached to adapters derived from a plasma sample can be incorporated into a DNA sequencing library. Sequencing libraries from several plasma samples can be pooled with control samples, purified, and, in some embodiments, sequenced on Illumina sequencers using a 75-cycle single-end, dual index sequencing kit. Primary sequencing output can be demultiplexed followed by quality trimming of the reads. In some embodiments, the reads that pass quality filters are aligned against human and synthetic references and then excluded from the analysis, or otherwise set aside. Reads potentially representing human satellite DNA can also filtered, e.g., via a k-mer-based method; then the remaining reads can be aligned with a microorganism reference database, (e.g., a database with 20,963 assemblies of high-quality genomic references). In some embodiments, reads with alignments that exhibit both high percent identity and/or high query coverage can be retained, except, e.g., for reads that are aligned with any mitochondrial or plasmid reference sequences. PCR duplicates can be removed based on their alignments. Relative abundances can be assigned to each taxon in a sample based on the sequencing reads and their alignments.

For each combination of read and taxon, a read sequence probability can be defined that accounts for the divergence between the microorganism present in the sample and the reference assemblies in the database. A mixture model can be used to assign a likelihood to the complete collection of sequencing reads that included the read sequence probabilities and the (unobserved) abundances of each taxon in the sample. In some cases, an expectation-maximization algorithm is applied to compute the maximum likelihood estimate of each taxon abundance. From these abundances, the number of reads arising from each taxon can be aggregated up the taxonomic tree. The estimated taxa abundances from the no template control (NTC) samples within the batch can be combined to parameterize a model of read abundance arising from the environment with variations driven by counting noise. Statistical significance values can then be computed for each estimate of taxon abundance in each patient sample. In some embodiments, taxa that exhibit a high significance level, and are one of the 1449 taxa within the reportable range, comprise the candidate calls. Final calls can be made after additional filtering is applied, which accounts for read location uniformity as well as cross-reactivity risk originating from higher abundance calls. The microorganism calls that pass these filters are reported along with abundances in MPM, as estimated using the ratio between the unique reads for the taxon and the number of observed unique reads of normalization molecules.

The amount of mcfDNA plasma concentration in each sample can then be quantified by using the measured relative abundance of the synthetic molecules initially spiked in the plasma.

Disclosed herein in some embodiments, are methods of analyzing nucleic acids. Such analytical methods include sequencing the nucleic acids as well as bioinformatic analysis of the sequencing results (e.g., sequence reads).

In some embodiments, a sequencing is performed using a next generation sequencing assay. As used herein, the term “next generation” generally refers to any high-throughput sequencing approach including, but not limited to one or more of the following: massively-parallel signature sequencing, pyrosequencing (e.g., using a Roche 454 GENOME ANALYZER™ sequencing device), ILLUMINA™ (SOLEXA™) sequencing (e.g., using an Illumina NEXTSEQ™ 500), sequencing by synthesis (ILLUMINA™), ion semiconductor sequencing (Ion Torrent™), sequencing by ligation (e.g., SOLiD™ sequencing), single molecule real-time (SMRT) sequencing (e.g., PACIFIC BIOSCIENCE™), polony sequencing, DNA nanoball sequencing (COMPLETE GENOMICS™), heliscope single molecule sequencing (Helicos Biosciences™), metagenomic sequencing and nanopore sequencing (e.g., OXFORD NANOPORE™). In some embodiments, a sequencing assay can comprise nanopore sequencing. In some embodiments, a sequencing assay can include some form of Sanger sequencing. In some embodiments, a sequencing can involve shotgun sequencing; in some embodiments, a sequencing can include bridge amplification PCR.

In some embodiments, a sequencing assay comprises a Gilbert's sequencing method. In some embodiments, a Gilbert's sequencing method can comprise chemically modifying nucleic acids (e.g., DNA) and then cleaving them at specific bases. In some embodiments, a sequencing assay can comprise dideoxynucleotide chain termination or Sanger-sequencing.

In some embodiments, a sequencing-by-synthesis approach is used in the methods provided herein. In some embodiments, fluorescently labeled reversible-terminator nucleotides are introduced to clonally-amplified DNA templates immobilized on the surface of a glass flowcell. During each sequencing cycle, a single labeled deoxynucleoside triphosphate (dNTP) may be added to the nucleic acid chain. The labeled terminator nucleotide may be imaged when added in order to identify the base and then the terminator group may be enzymatically cleaved to allow synthesis of the strand to proceed. A terminator group can comprise a 3′-O-blocked reversible terminator or a 3′-unblocked reversible terminator. Since all four reversible terminator-bound dNTPs (A, C, T, G) are generally present as single, separate molecules, natural competition may minimize incorporation bias.

In some embodiments, a method called Single-molecule real-time (SMRT) is used. In such approach, nucleic acids (e.g., DNA) are synthesized in zero-mode waveguides (ZMWs), which are small well-like containers with capturing tools located at the bottom of the well. The sequencing is performed with use of unmodified polymerase (attached to the ZMW bottom) and fluorescently labelled nucleotides flowing freely in the solution. The fluorescent label is detached from the nucleotide upon its incorporation into the DNA strand, leaving an unmodified DNA strand. A detector such as a camera may then be used to detect the light emissions; and the data may be analyzed bioinformatically to obtain sequence information.

In some embodiments, a sequencing by ligation approach is used to sequence the nucleic acids in a sample. One example is the next generation sequencing method of SOLiD (Sequencing by Oligonucleotide Ligation and Detection) sequencing (Life Technologies). This next generation technology may generate hundreds of millions to billions of small sequence reads at one time. The sequencing method may comprise preparing a library of DNA fragments from the sample to be sequenced. In some embodiments, the library is used to prepare clonal bead populations in which only one species of fragment is present on the surface of each bead (e.g., magnetic bead). The fragments attached to the magnetic beads may have a universal P1 adapter sequence attached so that the starting sequence of every fragment is both known and identical. In some embodiments, the method may further involve PCR or emulsion PCR. For example, the emulsion PCR may involve the use of microreactors containing reagents for PCR. The resulting PCR products attached to the beads may then be covalently bound to a glass slide. A sequencing assay such as a SOLiD sequencing assay or other sequencing by ligation assay may include a step involving the use of primers. Primers may hybridize to the P1 adapter sequence or other sequence within the library template. The method may further involve introducing four fluorescently labelled di-base probes that compete for ligation to the sequencing primer. Specificity of the di-base probe may be achieved by interrogating every first and second base in each ligation reaction. Multiple cycles of ligation, detection and cleavage may be performed with the number of cycles determining the eventual read length. In some embodiments, following a series of ligation cycles, the extension product can be removed, and the template can be reset with a primer complementary to the n−1 position for a second round of ligation cycles. Multiple rounds (e.g., 5 rounds) of primer reset may be completed for each sequence tag. Through the primer reset process, each base may be interrogated in two independent ligation reactions by two different primers. For example, a base at read position 5 can be assayed by primer number 2 in ligation cycle 2 and by primer number 3 in ligation cycle 1.

In some embodiments, a detection or quantification analysis of oligonucleotides can be accomplished by sequencing. In some embodiments, entire synthesized oligonucleotides can be detected via full sequencing of all oligonucleotides by e.g., Illumina HiSeq 2500™, including the sequencing methods described herein.

In some embodiments, the sequencing is accomplished through classic Sanger sequencing methods. Sequencing can also be accomplished using high-throughput systems some of which allow detection of a sequenced nucleotide immediately after or upon its incorporation into a growing strand, e.g., detection of sequence in real time or substantially real time. In some embodiments, high throughput sequencing generates at least 1,000, at least 5,000, at least 10,000, at least 20,000, at least 30,000, at least 40,000, at least 50,000, at least 100,000, or at least 500,000 sequence reads per hour. In some embodiments, each read is at least 50, at least 60, at least 70, at least 80, at least 90, at least 100, at least 120, or at least 150 bases per read. In some embodiments, each read is up to 2000, up to 1000, up to 900, up to 800, up to 700, up to 600, up to 500, up to 400, up to 300, up to 200, or up to 100 bases per read. Long read sequencing can include sequencing that provides a contiguous sequence read of longer than 500 bases, longer than 800 bases, longer than 1000 bases, longer than 1500 bases, longer than 2000 bases, longer than 3000 bases, or longer than 4500 bases per read.

In some embodiments, a high-throughput sequencing can involve the use of technology available by Illumina's Genome Analyzer IIX™, MiSeq personal Sequencer™, or HiSeq™ systems, such as those using HiSeq 2500™, HiSeq 1500™, HiSeq 2000™, or HiSeq 1000™ These machines use reversible terminator-based sequencing by synthesis chemistry. These machines can sequence 200 billion or more reads in eight days. Smaller systems may be utilized for runs within 3, 2, or 1 days or less time. Short synthesis cycles may be used to minimize the time it takes to obtain sequencing results.

In some embodiments, a high-throughput sequencing involves the use of technology available by ABI Solid System. This genetic analysis platform can enable massively parallel sequencing of clonally amplified DNA fragments linked to beads. The sequencing methodology is based on sequential ligation with dye-labeled oligonucleotides.

In some embodiments, a next-generation sequencing can comprise ion semiconductor sequencing (e.g., using technology from Life Technologies™ (Ion Torrent™)). Ion semiconductor sequencing can take advantage of the fact that when a nucleotide is incorporated into a strand of DNA, an ion can be released. To perform ion semiconductor sequencing, a high-density array of micromachined wells can be formed. Each well can hold a single DNA template. Beneath the well can be an ion sensitive layer, and beneath the ion sensitive layer can be an ion sensor. When a nucleotide is added to a DNA, an H+ ion can be released, which can be measured as a change in pH. The H+ ion can be converted to voltage and recorded by the semiconductor sensor. An array chip can be sequentially flooded with one nucleotide after another. In some embodiments, no scanning, light, or cameras are required. In some embodiments, an IONPROTON™ Sequencer is used to sequence nucleic acid. In some embodiments, an IONPGM™ Sequencer is used. The Ion Torrent Personal Genome Machine™ (PGM) can sequence 10 million reads in two hours.

In some embodiments, a high-throughput sequencing involves the use of technology available by Helicos BioSciences Corporation™ (Cambridge, Massachusetts) such as the Single Molecule Sequencing by Synthesis (SMSS) method. SMSS can allow for sequencing the entire human genome in up to 24 hours. In some embodiments, SMSS may not require a pre amplification step prior to hybridization. In some embodiments, SMSS may not require any amplification. In some embodiments, methods of using SMSS are described in part in US Publication Application Nos. 20060024711; 20060024678; 20060012793; 20060012784; and 20050100932, each of which are herein incorporated by reference.

In some embodiments, a high-throughput sequencing involves the use of technology available by 454 Lifesciences, Inc.™ (Branford, Connecticut) such as the Pico Titer Plate™ device which includes a fiber optic plate that transmits chemiluminescent signal generated by the sequencing reaction to be recorded by a charge-coupled device (CCD) camera in the instrument. This use of fiber optics can allow for the detection of a minimum of 20 million base pairs in 4.5 hours. In some embodiments, methods for using bead amplification followed by fiber optics detection are described in Marguiles, M., et al. “Genome sequencing in microfabricated high-density picolitre reactors”, Nature, doi: 10.1038/nature03959; which is herein incorporated by reference.

In some embodiments, high-throughput sequencing is performed using Clonal Single Molecule Array (Solexa, Inc.™) or sequencing-by-synthesis (SBS) utilizing reversible terminator chemistry. Methods of using these technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36, each of which are herein incorporated by reference.

In some embodiments, the next generation sequencing is nanopore sequencing. A nanopore can be a small hole, e.g., on the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies™; e.g., a GridION™ system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO²). The nanopore can be a hybrid pore (e.g., an integration of a protein pore into a solid-state membrane). The nanopore can be a nanopore with an integrated sensors (e.g., tunneling electrode detectors, capacitive detectors, or graphene-based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some embodiments, a nanopore sequencing technology from GENIA™ can be used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys™. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM™ or Roche™. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics™; see e.g., Drmanac et al. (2010) Science 327: 78-81, which is incorporated herein by reference). DNA can be isolated, fragmented, and size selected. For example, DNA can be fragmented (e.g., by sonication) to a mean length of about 500 bp. Adaptors (Ad1) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Ad1 adapter. A restriction enzyme (e.g., Acul) can be applied, and the DNA can be cleaved 13 bp to the left of the Ad1 to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize, and a single strand can fold onto itself to form a DNA nanoball (DNB™) which can be approximately 200-300 nanometers in diameter on average. A DNA nanoball can be attached (e.g., by adsorption) to a microarray (sequencing flow cell). The flow cell can be a silicon wafer coated with silicon dioxide, titanium and hexamethyldisilazane (HMDS) and a photoresistant material. Sequencing can be performed by unchained sequencing by ligating fluorescent probes to the DNA. The color of the fluorescence of an interrogated position can be visualized by a high-resolution camera. The identity of nucleotide sequences between adaptor sequences can be determined.

The methods provided herein may include use of a system that contains a nucleic acid sequencer (e.g., DNA sequencer, RNA sequencer) for generating DNA or RNA sequence information. The system may include a computer comprising software or code that performs bioinformatic analysis on the DNA or RNA sequence information. Bioinformatic analysis can include, without limitation, assembling sequence data, detecting and quantifying genetic variants in a sample, including germline variants and somatic cell variants (e.g., a genetic variation associated with cancer or pre-cancerous condition, a genetic variation associated with infection, or a combination thereof). In some embodiments, the bioinformatic analysis determines the threshold value for an assay provided herein, such as a method of determining a response to treatment. In some cases, the bioinformatics analysis further compares the value obtained in a longitudinal sample against the threshold value in order to determine whether there is a response to treatment. In some cases, the threshold value is determined in terms of MPM. In some cases, the bioinformatics analysis applies a known threshold, such as a known threshold value for a particular condition or microbe. For example, in some cases the threshold varies depending on whether an endocarditis patient has a native or prosthetic valve. More specifically, in some embodiments, the threshold value of MPM for the prosthetic valve is higher than that of the native value. In some cases, the bioinformatics analysis uses a program that recognizes and applies different MPM thresholds depending on the condition of the patient (e.g., prosthetic valve, native valve, endocarditis, pneumonia), or the type of microbe.

Sequencing data may be used to determine genetic sequence information, ploidy states, the identity of one or more genetic variants, as well as a quantitative measures of the variants, including relative and absolute relative measures.

In some embodiments a sequencing can involve sequencing of a genome. In some embodiments a genome can be that of a microbe or pathogen as disclosed herein. In some embodiments, sequencing of a genome can involve whole genome sequencing or partial genome sequencing. In some embodiments, a sequencing can be unbiased and can involve sequencing all or substantially all (e.g., greater than 70%, 80%, 90%) of the nucleic acids in a sample. In some embodiments, a sequencing of a genome can be selective, e.g., directed to portions of a genome of interest. In some embodiments, sequencing of select genes, or portions of genes may suffice for a desired analysis. In some embodiments, polynucleotides mapping to specific loci in a genome can be isolated for sequencing by, for example, sequence capture or site-specific amplification.

In some embodiments, the methods comprise detection of one or more cfDNAs from microbial cfDNAs (mcfDNAs), donor cfDNA, or subject cfDNA. In some embodiments, the methods comprise detection of multiple mcfDNAs (e.g., pCMV mcfDNA and/or S. aureus mcfDNA). In some embodiments, the subject cfDNA comprises human cfDNA. In some embodiments, the donor cfDNA comprises human cfDNA. In some embodiments, the donor cfDNA comprises non-human cfDNA. Non-limiting examples of donor cfDNA that is non-human includes porcine cfDNA, bovine cfDNA, equine cfDNA, non-human primate cfDNA, caprine cfDNA, and ovine cfDNA. Non-limiting examples of non-human primates includes chimpanzees, baboons, Rhesus monkeys, orangutans, macaques, and gorillas.

In some embodiments, the methods comprise detection of both mcfDNAs (e.g., pCMV mcfDNA) and human cfDNA. In some embodiments, the method involves detection and/or monitoring of a population of ˜55 nt mcfDNA (e.g., pCMV mcfDNA) and/or ˜55 nt human cell-free DNA. In some embodiments, a progressive increase in pCMV and human sequencing reads derived from the ˜55 nt population through a treatment regimen is an indication of a single biological process governing the progressively shifting distribution of the cfDNA fragment sizes; for example, a host immune response to pCMV-infected human cells. In some cases, the relative fraction of 55 nt fragments is correlated with changes in viral replication activity. In some cases, the method may comprise performing a PCR (qPCR) assay on mcfDNA (e.g., pCMV mcfDNA) using relatively shorter amplicon lengths than of 50-350 bp. In some cases, the amplicon length is 20, 30, 40, 50, 55, 60, 70, 80, 90, or 100 bp. In some cases, the amplicon length is at least 20, 30, 40, 50 or 55 base pairs. In some cases, the amplicon length is no more than 40, 50, 55, 60, 70, 80, or 90 base pairs.

In some embodiments, sensitivity of a test refers to a test's ability to correctly detect subjects with an infection who have an infection. In some embodiments, a sensitivity is a detection rate of a disease or infection. In some embodiments, a sensitivity is the proportion of people who test positive for a disease among those who have the disease. In some embodiments, a sensitivity can be calculated using the following formula: Sensitivity=(number of true positives)/(number of true positives+number of false negatives) or Sensitivity=(number of true positives)/(total number of sick individuals in a population); or Sensitivity=probability of a true positive.

In some embodiments, a specificity can refer to a test's ability to correctly reject healthy subjects without an infection. In some embodiments, a specificity of a test can comprise a proportion of subjects who truly do not have an infection who test negative for the infection. In some embodiments, a specificity can be calculated using the following formula: Specificity=(number of true negatives)/(number of true negatives+number of false positives) or Specificity=(number of true negatives)/(total number of well individuals in a population); or Specificity=probability of a negative test when the patient is healthy or well. In some cases, specificity is the proportion of negative control samples for which no bacterial or fungal organisms were identified by mcfDNA sequencing.

In some embodiments, the quantity for each organism identified in a method provided herein is expressed in Molecules Per Microliter (MPM), the number of DNA sequencing reads from the reported organism present per microliter of plasma. In some cases, detection or prediction of infection (or prediction of onset of symptoms of infection) occurs when the MPM is greater than a threshold value. In some cases, such threshold value of MPM may be greater than about 10, 15, 20, 30, 50, 100, 200, 300, 400, 500, 600, 700, 800, 900, 1000, 2000, 5000, 7000, 10000, 20000, 30000, or 40000. In some cases, the MPM threshold is determined for a particular organism.

In some embodiments, the quantity for a microbe (e.g., bacterium, fungus, virus) identified in a method provided herein is expressed as the amount or quantity of the microbe in a sample in relation to, or compared with, a threshold value, e.g., the amount of microbial cell-free nucleic acid in a sample as a percentage of the amount of the microbial cell-free nucleic acid in an initial sample. In some cases, the threshold value is an absolute value that can be used generally, irrespective of the subject. For example, the threshold value may be a normalized value signifying an average MPM value for a particular microbe in samples from a cohort of infected individuals prior to starting treatment for the infection. In some embodiments, the threshold value is the amount of a microbe measured in the initial sample (e.g., plasma, serum, cell-free sample) that is collected from the patient before beginning the treatment regimen for the microbial infection or while the patient is undergoing the treatment regimen for the microbial infection (e.g., in the initial stages of undergoing such treatment regimen). The amount of a microbe may be based on measurements of microbial cell-free nucleic acid (mcfNA). The mcfNA may be microbial cell-free DNA (mcfDNA). The amount of mcfNA may be expressed in MPM. In some cases, the MPM is an adjusted or normalized value. For example, the MPM may be adjusted based on the quantity of synthetic nucleic acids detected.

As used herein, a sample collected after an initial sample is a “longitudinal sample” or “longitudinal plasma sample.” In some cases, the MPM threshold is determined for a particular microbe. For example, the microbe can be the microbe associated with the microbial infection of the patient. In some cases, the amount of the mcfNA (e.g., MPM) compared to a threshold value may indicate a subject's response to a treatment. In some cases, a response to treatment is indicated when the amount of mcfNA in the longitudinal plasma sample is 10%-100% lower than the threshold value, or the amount of mcfNA in the longitudinal plasma sample is 25%-100% lower than the threshold value, or the amount of mcfNA in the longitudinal plasma sample is 50%-100% lower than the threshold value, or the amount of mcfNA in the longitudinal plasma sample is 75%-100% lower than the threshold value. In some cases, a response to treatment is indicated when the amount of mcfNA in the longitudinal plasma sample is at least about 10%, 20%, 25%, 30%, 35%, 40%, 45%, 50%, 55%, 70%, 80%, 90% or 100% lower than the threshold value. By way of example, if the threshold value is 100 MPM, an MPM of 25 for a longitudinal sample indicates a value that is 75% lower than the threshold value.

### Aligning Sequencing Reads

Following sequencing, the dataset of sequences can be uploaded to a data processor for bioinformatics analysis to subtract host sequences, e.g., human, cat, dog, etc. from the analysis; and determine the presence and prevalence of pathogen or contaminant sequences (for example microbial sequences), for example by a comparison of the coverage of sequences mapping to a microbial reference sequence to coverage of the host reference sequence. The subtraction of host sequences may include the step of identifying a reference host sequence, and masking microbial sequences or microbial-mimicking sequences present in the reference host genome. Similarly, determining the presence of a microbial sequence by comparison to a microbial reference sequence may include the step of identifying a reference microbial sequence, and masking host sequences or host-mimicking sequences present in the reference microbial genome.

Following sequencing, the dataset of sequences can be uploaded to a data processor for bioinformatics analysis to separate host sequences (e.g., human, cat, dog), donor sequences (e.g., pig, cow, horse, sheep, non-human primate), and microbial sequences.

The dataset can be optionally cleaned to check sequence quality, remove remnants of sequencer specific nucleotides (adapter sequences), and merge paired end reads that overlap to create a higher quality consensus sequence with less read errors. Repetitive sequences can be identified as those having identical start sites and length, and duplicates may be removed from the analysis.

In some aspects, human sequences can be subtracted from the analysis. In some aspects, the amplification/sequencing steps can be unbiased and the preponderance of sequences in a sample will be host sequences. The subtraction process may be optimized in several ways to improve the speed and accuracy of the process, for example by performing multiple subtractions where the initial alignment is set at a coarse filter, e.g., with a fast aligner, and performing additional alignments with a fine filter such as. a sensitive aligner.

The database of reads can be initially aligned against a human reference genome, including without limitation Genbank hg19 or Genbank hg38 reference sequences, to bioinformatically subtract the host DNA. Each sequence can be aligned with the best fit sequence in the human reference sequence. Sequences positively identified as human can be bioinformatically removed from the analysis.

The reference human sequence can also be optimized by adding in contigs that have a high hit rate, including without limitation highly repetitive sequence present in the genome that are not well represented in reference databases. It has been observed that of the reads that do not align to hg19 or hg38, a significant amount is eventually identified as human in a later stage of the pipeline, when a database that includes a large set of human sequences is used, for example the entire NCBI NT database. Removing these reads earlier in the analysis can be performed by building an expanded human reference. This reference can be created by identifying human contigs in a human sequence database other than the reference, e.g., NCBI NT database, that have high coverage after the initial human read subtraction. Those contigs can be added to the human reference to create a more comprehensive reference set. Additionally, novel assembled human contigs from cohort studies can be used as a further mask for human-derived reads.

In some cases, regions of the human genome reference sequence that contain non-human sequences may be masked, e.g., viral and bacterial sequences that are integrated into the genome of the reference sample. Sequence reads identified as non-human can then be aligned to a nucleotide database of microbial reference sequences. The database may be selected for those microbial sequences known to be associated with the host, e.g., the set of human commensal and pathogenic microorganisms. The microbial cfNA sequence reads may be aligned to the genome of a microbe. The microbial cfNA sequence reads may be aligned to the genome of a bacterium. The microbial cfNA sequence reads may be aligned to the genome of a virus. The microbial cfNA sequence reads may be aligned to a cytomegalovirus (CMV) genome. The CMV genome may be a porcine CMV genome. The CMV genome may be a bovine CMV genome. The CMV genome may be an equine CMV genome. The CMV genome may be a human CMV genome.

The microbial database may be optimized to mask or remove contaminating sequences. For example, many public database entries include artifactual sequences not derived from the microorganism, e.g., primer sequences, host sequences, and other contaminants. It may be desirable to perform an initial alignment or plurality of alignments on a database. Regions that show irregularities in read coverage when multiple samples are aligned can be masked or removed as an artifact. The detection of such irregular coverage can be done by various metrics, such as the ratio between coverage of a specific nucleotide and the average coverage of the entire contig within which this nucleotide is found. In general, a sequence that is represented as greater than about 5×, about 10×, about 25×, about 50×, about 100× the average coverage of that reference sequence can be artifactual. Alternatively, a binomial test can be applied to provide a per-base likelihood of coverage given the overall coverage of the contig. Removal of contaminant sequence from reference databases allows accurate identification of microbes.

In some embodiments, the method further comprises aligning mcfNA sequence reads. The mcfNA sequence reads may be aligned using a reference sequence of the one or more microbe. The reference sequence of a microbe may be a complete genome of the one or more microbe. The reference sequence of a microbe may be a partial genome of the one or more microbe. The reference sequence of a microbe may cover the origin of replication of the genome of the one or more microbe. The reference sequence of a microbe may cover sequences surrounding the origin of replication.

Each high confidence read may align to multiple organisms in the given microbial database. To correctly assign organism abundance based upon this possible mapping redundancy, an algorithm can be used to compute the most likely organism (for example see Lindner et al. Nucl. Acids Res. (2013) 41 (1): e10). For example, GRAMMy or GASiC algorithms can be used to compute the most likely organism that a given read came from.

Alignments and assignment to a host sequence or to a non-host (e.g., microbial) sequence may be performed in accordance with art-recognized methods. For example, a read of 50 nt. may be assigned as matching a given genome if there is not more than 1 mismatch, not more than 2 mismatches, not more than 3 mismatches, not more than 4 mismatches, not more than 5 mismatches, etc. over the length of the read. Commercial algorithms are generally used for alignments and identification. A non-limiting example of such an alignment algorithm is the bowtie2 program (Johns Hopkins University).

These assignments of reads to an organism (e.g., host organism, non-host organism, microbe, pathogen, etc.) can then totaled and used to compute the estimated number of reads assigned to each organism in a given sample or sample of a dilution series, in a determination of the presence or quantity of the organism in the sample (for example, a cell-free nucleic acid sample) or each dilution of a dilution series. This information can be used to determine an origin of a pathogen or contaminant. The analysis can normalize the counts for the size of the microbial genome to provide a calculation of coverage for the microbe. The normalized coverage for each microbe can be compared to the host sequence coverage in the same sample to account for differences in sequencing depth between samples.

Further, a dataset of microbial organisms represented by sequences in the sample, and the prevalence of those microorganisms can be optionally aggregated and displayed for ready visualization, e.g., in the form of a report.

In some embodiments, the analysis disclosed herein can be used to compute a pathogenicity score, where the pathogenicity score is a numeric or alphabetic value that summarizes the overall pathogenicity of the organism for ease of interpretation, e.g., by a health practitioner. Different microbes present in the microbiome may be assigned different scores. The final “pathogenicity score” can be a combination of many different factors, and typically provided as an arbitrary unit, for example ranging from 0-1, 0-10, or 0-100; as a percentile from all observed pathogenicity scores for a microbe of interest, etc. The specific parameters and weights of those parameters may be determined experimentally, e.g., by fitting the function to observed disease severity, or manually by setting the importance of different parameters and criteria.

Factors relevant for calculation of a pathogenicity score may include, without limitation, abundance of the microbe, e.g., as computed by number of reads relative to human reads, relative to the abundance of the microbe reads, as well as any of elevated coverage surrounding the origin of replication, diversity of fragment lengths, locations, compositions, and strand biases. Specific mutations found in the microbe genome, which may be made with reference to a database including SNPs, indels, and plasmids can allow for association of a microbe with toxicity, pathogenicity, and antimicrobial resistance. In the case of bacteria, expression of certain patterns of coverage over the genome for cfDNA (which can show great bias towards the origin of replication during rapid division) can be relevant to the pathogenicity score and/or indicative of whether a microbe is actively replicating or is in a latent phase.

In some embodiments, the methods disclosed herein inform replication kinetics for one or more microbes. In some embodiments, the replication kinetics may coincide with the pathogenicity score. The replication kinetics may be informed by read coverage surrounding the origin of replication, diversity of fragment lengths, locations, composition, as well as strand biases. For example, higher read coverage at or near the origin of replication as compared to reads covering other regions especially regions near the termini could indicate active replication. In contrast, another example may have higher read coverage other regions other than at or near the origin of replication especially regions near the termini as compared to reads covering at or near the origin of replication could indicate reduced and/or diminishing replication.

### Amplification

Disclosed herein are methods for detecting an actively replicating microbe. Methods disclosed herein may include using targeted amplification of sequences near both the ORI and ter to infer replication of a microbe. Disclosed herein in some embodiments, are methods of determining a PTR score using PCR amplification as disclosed herein. In some embodiments, a targeted amplification may use microbial cell-free DNA (mcfDNA). In some embodiments, PCR can be performed on cell-free nucleic acids (cfNA) in a biological sample to generate microbial cell-free nucleic acid amplifications.

Provided herein are methods for detecting the ratio of sequences at or near the origin of replication (ORI) as compared to sequences at or near the replication terminus (ter). In some embodiments, determining a PTR score using PCR amplification can comprise using targeted amplification of DNA proximal to and distant from the origin of replication to infer microbial replication. In some embodiments, a microbe can be detected from a biological sample of a subject (e.g., a transplant recipient). In some embodiments, a microbe can be analyzed using cfNA from a microbe.

In some embodiments, PCR can comprise PCR, qPCR, digital PCR, or any combination thereof as disclosed herein. In some embodiments, an amplification can comprise traditional PCR. In some embodiments, a traditional PCR product can undergo sequencing, such as in target or amplicon sequencing. In some embodiments, methods disclosed herein are performed without sequencing. In some embodiments, an amplification may use a reporter, for example as in qPCR or microarray. In some embodiments, cfNA of a microbe can be analyzed using quantitative polymerase chain reaction (qPCR) and primers specific for a replicating microbe. In some embodiments, primers can be designed to produce amplicons that span at least a portion of an origin of replication of a microbe. In some embodiments, primers can be designed to produce amplicons that span a portion near an origin of replication of a microbe.

In some embodiments, a series of tiled PCR primers may be designed to amplify mcfDNA fragments that originated at and/or near the ORI and the ter. In some embodiments, the fragments that originated near the ORI and the ter are within 0-5 kb of the ORI or ter. In some embodiments, the amplification target will be within 50 bp of the origin of replication. In some embodiments, the capture of short amplicons may utilize PCR. In some embodiments, the short amplicons may be captured even when a primer ligates to mcfDNA away from the end of the fragment. The relative ratio of amplicons from these two regions (e.g., ORI and ter) may be used to infer the rate of DNA replication of a microbe.

A high ORI:ter ratio may indicate an active infection. A low ORI:ter ratio may indicate a latent infection. In some embodiments, multiple PCR targets may be used near the ORI and near the ter. The use of multiple PCR targets may allow for robust and/or sensitive detection. In some embodiments, the locations of PCR primers can be varied. In some embodiments, the strand orientation of the PCR primers can be varied. In some embodiments, the strandedness may be used as another component to determine latent verses active replication of a microbe. In some embodiments, primers can be designed to span either individual or multiple ORI & ter sites in species with more than one ORI. In some embodiments, the amplicons generated as described herein may be used to create a sequencing library.

In some embodiments, a method can further comprise quantitatively amplifying mcfNA to determine a quantity of mcfNA at a genomic locus of a microbial genome. In some embodiments, a genomic locus can comprise a position spanning at least a portion of or a position proximal to an ORI or ter. In some embodiments, a genomic locus can comprise a position that does not span at least a portion of or that is proximal to an ORI.

In some embodiments, a quantity of mcfNA amplifications spanning at least a portion of or proximal to an ORI or ter can be determined. In some embodiments a quantity of mcfNA amplifications that do not span at least a portion of and are not proximal to an ORI can be determined. In some embodiments, determining a quantity of mcfNA amplifications at a locus can be used to determine a quantity of mcfNA derived from a specific locus on a microbial genome relative to the quantity of mcfNA that was derived from the remainder of the microbial genome. In some embodiments, determining a quantity of mcfNA amplifications at a locus can be used to determine a quantity of mcfNA derived from a specific locus on a microbial genome relative to the quantity of mcfNA that was derived from a second locus of the microbial genome.

### Origin of Replication, Peak-to-Trough Ratio (PTR)

The methods provided herein, in some embodiments, include methods of identifying active replication of a microbe in a subject, such as a human subject. In some embodiments, the methods comprise quantifying an amount of microbial cell-free nucleic acids (mcfNA) within an origin of replication region of a microbial genome. An origin of replication is generally a particular sequence in a genome at which replication is initiated. In some microbial genomes, there may be a single origin of replication. The specific structure of the origin of replication varies somewhat from species to species, but most share some common characteristics such as high AT content (adenine and thymine). Other common characteristics present at or around the origin of replication includes a symmetric location and/or a palindromic or imperfect palindromic structure. In bacteria, the origin of replication binds the pre-replication complex, a protein complex that recognizes, unwinds, and begins to copy DNA. In viruses, host transcription machinery is used for replication. Most microbes have a single origin of replication. In some cases, the replication terminus is positioned approximately opposite the origin of replication on the circular microbial genome. In some cases, the replication terminus is positioned at the end of the linear microbial genome and may comprise terminal repeats.

Active replication may be detected using sequencing of microbial cfDNA, for example microbial cfDNA from plasma. In a population of replicating microbes, DNA coverage within an origin of replication region may be higher than DNA coverage at a replication terminus, or later-replicating regions of the genome. Such imbalance between the origin or replication region and the replication terminus region may indicate active replication or infection.

The origin of replication region may comprise sequences proximal to the origin of replication. In some cases, the origin of replication region spans the origin of replication. In some cases, the origin of replication region comprises sequences proximal to the origin of replication but does not span the origin of replication. The methods provided herein may also include detection of sequences within a replication terminus region or origin of replication region of a microbial genome. In some cases, the methods comprise comparing coverage within an origin of replication region to coverage within a replication terminus region.

In some embodiments, a method can further comprise determining whether a microbe is actively replicating in a subject based on a coverage of an mcfNA near an origin of replication or within an origin of replication region. In some embodiments, a method can further comprise comparing a coverage of mcfNA near an origin of replication of a microbe to coverage in later replicating regions to derive a peak-to-trough ratio (PTR) score. In some embodiments, a method can further comprise comparing a coverage of mcfNA near an origin of replication of a microbe to coverage near a replication terminus (ter) to derive a peak-to-trough ratio (PTR) score. In some embodiments, a peak can comprise a number of reads that span at least a portion of an origin of replication (ORI) or a locus in proximity to an origin of replication. In some embodiments, a trough can comprise a number of reads that span at least a portion of a terminus (ter) or a locus in proximity to a terminus.

In a non-replicating organism, the expectation would be that sequencing reads are equally likely to be detected across all regions of a genome. In some embodiments, the methods provided herein apply a statistical test that interrogates whether observed coverage at an origin of replication (or origin of replication region) compared to coverage at the terminus is not equivalent, or is asymmetric. An observed increase in coverage at the origin of replication versus other genomic regions can be indicative of active replication.

In some embodiments, the methods may be performed using fragments of mcfDNA. The fragments of cfDNA may span the origin of replication. The fragments of cfDNA may span at least a portion of the origin of replication. The fragments may span at least a region proximal to the origin of replication. Non-limiting examples of regions considered proximal to the origin of replication include regions that are 0-50 kb downstream or upstream of the origin of replication. Non-limiting examples of regions considered proximal to the origin of replication include regions that are 0-5 kb downstream or upstream of the origin of replication. In some embodiments, the region proximal or near to the origin of replication or within an origin of replication region is at least about 0 kb, at least about 0.1 kb, at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 1.9 kb, at least about 2 kb, at least about 2.1 kb, at least about 2.2 kb, at least about 2.3 kb, at least about 2.4 kb, at least about 2.5 kb, at least about 2.6 kb, at least about 2.7 kb, at least about 2.8 kb, at least about 2.9 kb, at least about 3 kb, at least about 3.1 kb, at least about 3.2 kb, at least about 3.3 kb, at least about 3.4 kb, at least about 3.5 kb, at least about 3.6 kb, at least about 3.7 kb, at least about 3.8 kb, at least about 3.9 kb, at least about 4 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, at least about 4.9 kb, at least about 5 kb, at least about 5.1 kb, at least about 5.2 kb, at least about 5.3 kb, at least about 5.4 kb, at least about 5.5 kb, at least about 5.6 kb, at least about 5.7 kb, at least about 5.8 kb, at least about 5.9 kb, at least about 6 kb, at least about 6.1 kb, at least about 6.2 kb, at least about 6.3 kb, at least about 6.4 kb, at least about 6.5 kb, at least about 6.6 kb, at least about 6.7 kb, at least about 6.8 kb, at least about 6.9 kb, at least about 7 kb, at least about 7.1 kb, at least about 7.2 kb, at least about 7.3 kb, at least about 7.4 kb, at least about 7.5 kb, at least about 7.6 kb, at least about 7.7 kb, at least about 7.8 kb, at least about 7.9 kb, at least about 8 kb, at least about 8.1 kb, at least about 8.2 kb, at least about 8.3 kb, at least about 8.4 kb, at least about 8.5 kb, at least about 8.6 kb, at least about 8.7 kb, at least about 8.8 kb, at least about 8.9 kb, at least about 9 kb, at least about 9.1 kb, at least about 9.2 kb, at least about 9.3 kb, at least about 9.4 kb, at least about 9.5 kb, at least about 9.6 kb, at least about 9.7 kb, at least about 9.8 kb, at least about 9.9 kb, at least about 10 kb, at least about 15 kb, at least about 20 kb, at least about 25 kb, at least about 30 kb, at least about 35 kb, at least about 40 kb, at least about 45 kb, or at least about 50 kb from either side of the origin of replication. In some embodiments, the region proximal or near to the origin of replication is at most about 0 kb, at most about 0.1 kb, at most about 0.2 kb, at most about 0.3 kb, at most about 0.4 kb, at most about 0.5 kb, at most about 0.6 kb, at most about 0.7 kb, at most about 0.8 kb, at most about 0.9 kb, at most about 1 kb, at most about 1.1 kb, at most about 1.2 kb, at most about 1.3 kb, at most about 1.4 kb, at most about 1.5 kb, at most about 1.6 kb, at most about 1.7 kb, at most about 1.8 kb, at most about 1.9 kb, at most about 2 kb, at most about 2.1 kb, at most about 2.2 kb, at most about 2.3 kb, at most about 2.4 kb, at most about 2.5 kb, at most about 2.6 kb, at most about 2.7 kb, at most about 2.8 kb, at most about 2.9 kb, at most about 3 kb, at most about 3.1 kb, at most about 3.2 kb, at most about 3.3 kb, at most about 3.4 kb, at most about 3.5 kb, at most about 3.6 kb, at most about 3.7 kb, at most about 3.8 kb, at most about 3.9 kb, at most about 4 kb, at most about 4.1 kb, at most about 4.2 kb, at most about 4.3 kb, at most about 4.4 kb, at most about 4.5 kb, at most about 4.6 kb, at most about 4.7 kb, at most about 4.8 kb, at most about 4.9 kb, at most about 5 kb, at most about 5.1 kb, at most about 5.2 kb, at most about 5.3 kb, at most about 5.4 kb, at most about 5.5 kb, at most about 5.6 kb, at most about 5.7 kb, at most about 5.8 kb, at most about 5.9 kb, at most about 6 kb, at most about 6.1 kb, at most about 6.2 kb, at most about 6.3 kb, at most about 6.4 kb, at most about 6.5 kb, at most about 6.6 kb, at most about 6.7 kb, at most about 6.8 kb, at most about 6.9 kb, at most about 7 kb, at most about 7.1 kb, at most about 7.2 kb, at most about 7.3 kb, at most about 7.4 kb, at most about 7.5 kb, at most about 7.6 kb, at most about 7.7 kb, at most about 7.8 kb, at most about 7.9 kb, at most about 8 kb, at most about 8.1 kb, at most about 8.2 kb, at most about 8.3 kb, at most about 8.4 kb, at most about 8.5 kb, at most about 8.6 kb, at most about 8.7 kb, at most about 8.8 kb, at most about 8.9 kb, at most about 9 kb, at most about 9.1 kb, at most about 9.2 kb, at most about 9.3 kb, at most about 9.4 kb, at most about 9.5 kb, at most about 9.6 kb, at most about 9.7 kb, at most about 9.8 kb, at most about 9.9 kb, at most about 10 kb, at most about 15 kb, at most about 20 kb, at most about 25 kb, at most about 30 kb, at most about 35 kb, at most about 40 kb, at most about 45 kb, or at most about 50 kb from either side of the origin of replication. The fragments of cfDNA may span at least one replication terminus. The fragments of cfDNA may span at least a portion of at least one replication terminus. The fragments may span at least a region proximal to the at least one replication terminus. Non-limiting examples of regions considered proximal to the at least one replication terminus include regions that are 0-50 kb downstream or upstream of one or more replication termini. Non-limiting examples of regions considered proximal to the at least one replication terminus include regions that are 0-5 kb downstream or upstream of at least one replication terminus. In some embodiments, the region proximal or near to the termini is at least about 0 kb, at least about 0.1 kb, at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 1.9 kb, at least about 2 kb, at least about 2.1 kb, at least about 2.2 kb, at least about 2.3 kb, at least about 2.4 kb, at least about 2.5 kb, at least about 2.6 kb, at least about 2.7 kb, at least about 2.8 kb, at least about 2.9 kb, at least about 3 kb, at least about 3.1 kb, at least about 3.2 kb, at least about 3.3 kb, at least about 3.4 kb, at least about 3.5 kb, at least about 3.6 kb, at least about 3.7 kb, at least about 3.8 kb, at least about 3.9 kb, at least about 4 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, at least about 4.9 kb, at least about 5 kb, at least about 5.1 kb, at least about 5.2 kb, at least about 5.3 kb, at least about 5.4 kb, at least about 5.5 kb, at least about 5.6 kb, at least about 5.7 kb, at least about 5.8 kb, at least about 5.9 kb, at least about 6 kb, at least about 6.1 kb, at least about 6.2 kb, at least about 6.3 kb, at least about 6.4 kb, at least about 6.5 kb, at least about 6.6 kb, at least about 6.7 kb, at least about 6.8 kb, at least about 6.9 kb, at least about 7 kb, at least about 7.1 kb, at least about 7.2 kb, at least about 7.3 kb, at least about 7.4 kb, at least about 7.5 kb, at least about 7.6 kb, at least about 7.7 kb, at least about 7.8 kb, at least about 7.9 kb, at least about 8 kb, at least about 8.1 kb, at least about 8.2 kb, at least about 8.3 kb, at least about 8.4 kb, at least about 8.5 kb, at least about 8.6 kb, at least about 8.7 kb, at least about 8.8 kb, at least about 8.9 kb, at least about 9 kb, at least about 9.1 kb, at least about 9.2 kb, at least about 9.3 kb, at least about 9.4 kb, at least about 9.5 kb, at least about 9.6 kb, at least about 9.7 kb, at least about 9.8 kb, at least about 9.9 kb, at least about 10 kb, at least about 15 kb, at least about 20 kb, at least about 25 kb, at least about 30 kb, at least about 35 kb, at least about 40 kb, at least about 45 kb, or at least about 50 kb from at least one replication terminus. In some embodiments, the region proximal to the termini is at most about 0 kb, at most about 0.1 kb, at most about 0.2 kb, at most about 0.3 kb, at most about 0.4 kb, at most about 0.5 kb, at most about 0.6 kb, at most about 0.7 kb, at most about 0.8 kb, at most about 0.9 kb, at most about 1 kb, at most about 1.1 kb, at most about 1.2 kb, at most about 1.3 kb, at most about 1.4 kb, at most about 1.5 kb, at most about 1.6 kb, at most about 1.7 kb, at most about 1.8 kb, at most about 1.9 kb, at most about 2 kb, at most about 2.1 kb, at most about 2.2 kb, at most about 2.3 kb, at most about 2.4 kb, at most about 2.5 kb, at most about 2.6 kb, at most about 2.7 kb, at most about 2.8 kb, at most about 2.9 kb, at most about 3 kb, at most about 3.1 kb, at most about 3.2 kb, at most about 3.3 kb, at most about 3.4 kb, at most about 3.5 kb, at most about 3.6 kb, at most about 3.7 kb, at most about 3.8 kb, at most about 3.9 kb, at most about 4 kb, at most about 4.1 kb, at most about 4.2 kb, at most about 4.3 kb, at most about 4.4 kb, at most about 4.5 kb, at most about 4.6 kb, at most about 4.7 kb, at most about 4.8 kb, at most about 4.9 kb, at most about 5 kb, at most about 5.1 kb, at most about 5.2 kb, at most about 5.3 kb, at most about 5.4 kb, at most about 5.5 kb, at most about 5.6 kb, at most about 5.7 kb, at most about 5.8 kb, at most about 5.9 kb, at most about 6 kb, at most about 6.1 kb, at most about 6.2 kb, at most about 6.3 kb, at most about 6.4 kb, at most about 6.5 kb, at most about 6.6 kb, at most about 6.7 kb, at most about 6.8 kb, at most about 6.9 kb, at most about 7 kb, at most about 7.1 kb, at most about 7.2 kb, at most about 7.3 kb, at most about 7.4 kb, at most about 7.5 kb, at most about 7.6 kb, at most about 7.7 kb, at most about 7.8 kb, at most about 7.9 kb, at most about 8 kb, at most about 8.1 kb, at most about 8.2 kb, at most about 8.3 kb, at most about 8.4 kb, at most about 8.5 kb, at most about 8.6 kb, at most about 8.7 kb, at most about 8.8 kb, at most about 8.9 kb, at most about 9 kb, at most about 9.1 kb, at most about 9.2 kb, at most about 9.3 kb, at most about 9.4 kb, at most about 9.5 kb, at most about 9.6 kb, at most about 9.7 kb, at most about 9.8 kb, at most about 9.9 kb, at most about 10 kb, at most about 15 kb, at most about 20 kb, at most about 25 kb, at most about 30 kb, at most about 35 kb, at most about 40 kb, at most about 45 kb, or at most about 50 kb from at least one replication terminus.

The sequences surrounding the origin of replication may include sequences that cover the origin of replication. The sequences surrounding the origin of replication may be up to 50 kb from the origin of replication. The sequences surrounding the origin of replication may be up to 10 kb from the origin of replication. The sequences surrounding the origin of replication may be more than about 0.2 kb, 0.5 kb, 0.8 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 15 kb, 20 kb, 25 kb, 30 kb, 35 kb, 40 kb, 45 kb, or 50 kb. The sequences surrounding the origin of replication may be less than about 0.2 kb, 0.5 kb, 0.8 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 15 kb, 20 kb, 25 kb, 30 kb, 35 kb, 40 kb, 45 kb, 50 kb, or 55 kb. In some embodiments, the method further comprises determining the coverage of sequence reads from the mcfNA at or near the origin of replication of the microbe. The coverage of the sequence reads from the mcfNA at or near the origin of replication of the microbe may indicate whether the microbe is actively replicating. In some embodiments, the coverage of sequence reads from the mcfNA at or near the origin of replication of the microbe is compared to the coverage of sequencing reads near the termini. In some embodiments, the coverage of sequence reads from the mcfNA at or near the origin of replication of the microbe is compared to the coverage of sequencing reads distal to the origin of replication. In some embodiments, sequencing reads distal to the origin of replication are more than about 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or 200 kb from either side of the origin of replication. In some embodiments, sequencing reads distal to the origin of replication are at least about 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or 200 kb from either side of the origin of replication. In some embodiments, the coverage of sequence reads from the mcfNA at or near the origin of replication of the microbe is compared to the coverage of sequencing reads in later replicating regions. In some embodiments, sequencing reads in later replicating regions are more than about 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or 200 kb from either side of the origin of replication. In some embodiments, sequencing reads in later replicating regions are at least about 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or 200 kb from either side of the origin of replication. In some embodiments, sequencing reads in later replicating regions are at least about 0.2 kb, 0.5 kb, 0.8 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 15 kb, 20 kb, 25 kb, 30 kb, 35 kb, 40 kb, 45 kb, or 50 kb from one of the termini. In some embodiments, sequencing reads in later replicating regions are at most about 0.2 kb, 0.5 kb, 0.8 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 15 kb, 20 kb, 25 kb, 30 kb, 35 kb, 40 kb, 45 kb, 50 kb, or 50 kb from one of the termini. In some embodiments, the frequencies of the nucleotides may be displayed graphically as a function of their genomic location. In these graphs, a peak may coincide with the origin of replication or an origin of replication region and a trough may coincide with at least one replication terminus or replication terminus region.

In some embodiments, active replication can be identified when a PTR score is greater than 1. In some embodiments, active replication is identified when a PTR score is greater than 1.1, 1.2, 1.3. 1.5, 1.75, 2, 2.5, 3, or 5. In some embodiments, the PTR score is a statistically significant score. In some embodiments, active replication is identified when the PTR score is greater than 1 (or 1.1, 1.2, 1.3, 1.4, 1.5, 2, etc.) and wherein coverage at the within the origin of replication region is greater than 2 reads, 5 reads, 10 reads, 50 reads, 75 reads, 100 reads, 150 reads, 200 reads, 250 reads, 500 reads, 1000 reads, 1500 reads, 2000 reads, 2500 reads, 3000 reads, 3500 reads. In some embodiments, active replication is identified when the PTR score is greater than 1 (or 1.1, 1.2, 1.3, 1.4, 1.5, 2, etc.) and wherein coverage within the origin of replication region is greater than 2 reads, 5 reads, 10 reads, 50 reads, 75 reads, 100 reads, 150 reads, 200 reads, 250 reads, 500 reads, 1000 reads, 1500 reads, 2000 reads, 2500 reads, 3000 reads, 3500 reads and wherein coverage within the replication terminus region is greater than 2 reads, 5 reads, 10 reads, 50 reads, 75 reads, 100 reads, 150 reads, 200 reads, 250 reads, 500 reads, 1000 reads, 1500 reads, 2000 reads, 2500 reads, 3000 reads, 3500 reads.

In some cases, a PTR score can be monitored over time. In some embodiments, active replication indicative of a worsening infection can be identified when a PTR score increases over time. In some embodiments, active replication indicative of a persistent or worsening infection can be identified when a PTR score is stable or increases over a period of at least 2, 3, 4, 5, 10, 15, 20, 25, 30, 35, or 40 days. In some embodiments, active replication indicative of a persistent or worsening infection can be identified when a PTR score increases by at least 1.5-fold, 2-fold, 3-fold, 5-fold, or 10-fold over a period of days (e.g., 1, 2, 3, 5, 10, 15, 20, 30, 40, or 50 days). In some embodiments, active replication indicative of a persistent or worsening infection can be identified when a PTR score is stable or increases in a statistically significant manner over a period of time.

In some embodiments, active replication indicative of an improving infection can be identified when a PTR score decreases over a period of at least 2, 3, 4, 5, 10, 15, 20, 25, 30, 35, or 40 days. In some embodiments, active replication indicative of an improving infection can be identified when a PTR score decreases by at least 1.5-fold, 2-fold, 3-fold, 5-fold, or 10-fold over a period of days (e.g., 1, 2, 3, 5, 10, 15, 20, 30, 40, or 50 days). In some embodiments, active replication indicative of an improving infection can be identified when a PTR score decreases in a statistically significant manner over a period of time.

The coverage of mcfNA sequence reads at or near the origin of replication of the microbe may be monitored over time. In some embodiments, an increase in coverage of mcfNA sequence reads at or near the origin of replication of the microbe over time may indicate active replication of the microbe. In some embodiments, an increase in coverage of mcfNA sequence reads at or near the origin of replication of the microbe over time may indicate increasing replication of the microbe. In some embodiments, an increase in coverage of mcfNA sequence reads at or near the origin of replication of the microbe over time may indicate a treatment is not working. In some embodiments, a decrease in coverage of mcfNA sequence reads at or near the origin of replication of the microbe over time may indicate decreasing replication of the microbe. In some embodiments, a decrease in coverage of mcfNA sequence reads at or near the origin of replication of the microbe over time may indicate latency of the microbe. In some embodiments, a decrease in coverage of mcfNA sequence reads at or near the origin of replication of the microbe over time may indicate a treatment is working.

In some embodiments, a higher PTR score can indicate that a microbe can be actively replicating at a more rapid rate relative to a lower PTR score. In some embodiments, a PTR score can be used to determine whether a microbe is actively replicating in a human subject.

In some embodiments, more than one target is selected for simultaneous quantification; these targets can be located close to the origin of replication and/or can involve both directions of replication. In some embodiments, more than one target is selected for simultaneous quantification. In some cases, one or more of these targets is located close to the origin of replication and one or more targets is far from the origin of replication (e.g., near the termini).

By measuring the ratio of these targets at certain times, replication can be detected rapidly in some embodiments. In some embodiments, more than one target is selected for simultaneous quantification; these can be located close to the origin of replication in both directions. For example, in some cases, absolute levels of pCMV mcfDNA are monitored over time and compared with genomic loci near an origin of replication. In some cases, an increase in mcfDNA near the origin of replication is a prelude, or indication, of a subsequent increase in absolute pCMV mcfDNA, which can appear between 1-12 days following the observed activity near the origin of replication. In some cases, the increase in absolute pCMV mcfDNA can appear at least 1, 2, 3, 4, 5, 6, or 7 days, or at most 7, 9, 10, 11, 12, 13, 14, 15, or 18 days following the observed activity near the origin of replication. In some cases, the dynamics of absolute levels of microbial cell-free nucleic acids (e.g., pCMV mcfDNA) near an origin of replication, potentially in comparison to absolute levels of microbial cell-free nucleic acids (e.g., pCMV mcfDNA) near one or more termini, over time can together, or individually, provide an indication as to active infection, a cause of a transplant injury, as well as a response to treatment, such as treatment with an antiviral (e.g., anti-replication antiviral, ganciclovir, valacyclovir, cidofovir). In some cases, the methods provided herein comprise detecting mcfDNA strand asymmetry around the viral origin of replication. In some embodiments, such asymmetry may represent the capture of single-stranded DNA intermediates of DNA replication.

In some embodiments, an expectation for statistical testing is that there is no difference in sequencing coverage or amplification at the ORI and the ter. In some embodiments, a statistically significant deviation from this expectation indicates that a microbe is actively replicating. In some embodiments, a formula for PTR can be a ratio (ORI/Ter). In some embodiments, obtaining a PTR ratio is possible with any number of sequencing reads>3 (i.e., 2/1, 20/10, 5,000,000/2,500,000).

It will be appreciated that if the position of the origin of replication and the terminus are known, then, in some embodiments, the method may be carried out by analyzing the coverage (or frequency) at these positions. However, if the position of the origin of replication and the terminus are not known, the method may be performed such that essentially all (or the majority) of the nucleotides across the genome are analyzed. In this way, the position of the origin of replication and the terminus may be determined, as further described herein.

In some embodiments, the origin or replication is identified. The origin of replication may be identified using an algorithm. The algorithm may be a dimensionality reduction algorithm. Non-limiting examples of dimensionality algorithms include principal component analysis (PCA), factor analysis, linear discriminant analysis (LDA), and non-negative matrix factorization (NMF). For example, PCA may be used on mcfDNA coverage profiles to identify sites with elevated coverage. The sites with elevated coverage can be indicative of the origin of replication.

Principal component analysis (PCA) may be performed on mcfNA. Principal component analysis (PCA) may be performed on mcfNA sequence reads. PCA may be measured for mcfNA. PCA may be measured on mcfNA sequence reads. PCA may be measured for the longest sequence reads from mcfNA. PCA may be measured for the longest contiguous sequence reads from mcfNA. In some embodiments, PCA is measured for the longest contiguous sequence reads from mcfNA in the top 20%. In some embodiments, PCA is measured for the longest contiguous sequence reads from mcfNA in the top 15%. In some embodiments, PCA is measured for the longest contiguous sequence reads from mcfNA in the top 10%. In some embodiments, PCA is measured for the longest contiguous sequence reads from mcfNA in the top 5%. In some embodiments, PCA is measured for the longest contiguous sequence reads from mcfNA in the top 1%. The PCA may be compared to the PTR score. In some embodiments, the PCA is referred to as principal component (PC).

In some embodiments, the sequence reads of the mcfNA are used to determine a concentration of a microbe. The concentration of a microbe may be monitored over time. In some embodiments, the sequence reads are used to determine a quantity of the mcfNA. The quantity of a microbe may be monitored over time. The PTR score may be monitored over time.

### Fragmentomics

Disclosed herein in some embodiments are methods of determining active microbial replication (e.g., viral, bacterial) in a subject by profiling fragments of cfNA, identifying an amount of fragments of a specific length in a sample from the subject, and comparing the amount to an amount of cfNA of other lengths. In some embodiments, an increase in peak or in an amount of cfNA fragments with a median length of about 55 bp in a sample from a subject relative to cfNA fragments with a median length of about 140 bp can indicate that a microbe is actively replicating in a subject. In some embodiments, determining a quantity of fragments of specific size can be performed by high-throughput sequencing as disclosed herein. In some embodiments, a relative amount of two different fragments can be determined by a Pearson correlation coefficient. In some embodiments, the relative proportion of very short cfDNA fragments may be correlated with inferred replication and can be used as a DNA marker to monitor microbial replication.

In some embodiments, the length of the relatively short cfDNA fragments may be at most about 20 nucleotides (nt), at most about 25 nt, at most about 30 nt, at most about 35 nt, at most about 40 nt, at most about 45 nt, at most about 50 nt, at most about 55 nt, at most about 60 nt, at most about 65 nt, at most about 70 nt, at most about 75 nt, at most about 80 nt, at most about 85 nt, at most about 90 nt, at most about 95 nt, at most about 100 nt, at most about 110 nt, at most about 120 nt, at most about 130 nt, at most about 140 nt, at most about 150 nt, at most about 160 nt, at most about 170 nt, at most about 180 nt, at most about 190 nt, at most about 200 nt, at most about 220 nt, at most about 240 nt, at most about 260 nt, at most about 280 nt, at most about 300 nt, at most about 320 nt, at most about 340 nt, or at most about 360 nt. In some embodiments, the length of the relatively cfDNA fragments may be at least about 20 nucleotides (nt), at least about 25 nt, at least about 30 nt, at least about 35 nt, at least about 40 nt, at least about 45 nt, at least about 50 nt, at least about 55 nt, at least about 60 nt, at least about 65 nt, at least about 70 nt, at least about 75 nt, at least about 80 nt, at least about 85 nt, at least about 90 nt, at least about 95 nt, at least about 100 nt, at least about 110 nt, at least about 120 nt, at least about 130 nt, at least about 140 nt, at least about 150 nt, at least about 160 nt, at least about 170 nt, at least about 180 nt, at least about 190 nt, at least about 200 nt, at least about 220 nt, at least about 240 nt, at least about 260 nt, at least about 280 nt, at least about 300 nt, at least about 320 nt, at least about 340 nt, or at least about 360 nt.

Microbial replication may be identified through analysis of fragments of mcfNA. Analysis of fragments of mcfNA may include measuring fluctuations of mcfNA fragments of like sizes. In some embodiments, the fluctuations of mcfNA fragments may be analyzed over time. In some embodiments, the fluctuations of mcfNA fragments may be analyzed to assess a response to a treatment. In some embodiments, response to a treatment is determined to be decreasing an infection. In some embodiments, response to a treatment is determined to have little to no impact on an infection. In some embodiments, the fragments of mcfNA may include measuring fluctuations of mcfNA are from short, ssDNA-derived mcfDNA fragments. In some embodiments, the fragments of mcfNA analyzed are between 40 nt and 300 nt. In some embodiments, the fragments of mcfNA analyzed are between 40 nt and 280 nt. In some embodiments, the fragments of mcfNA analyzed are between 55 nt and 280 nt. In some embodiments, the fragments of mcfNA analyzed are between 50 nt and 150 nt. In some embodiments, the fragments of mcfNA analyzed are between 55 nt and 140 nt. In some embodiments, mcfNA fragments of a particular fragment length are analyzed. Analysis of mcfNA fragments of a particular fragment length may include measuring the abundance of mcfNA fragments of a particular fragment length. Analysis of mcfNA fragments of a particular fragment length may include measuring the relative abundance of mcfNA fragments of a particular fragment length. Analysis of mcfNA fragments of a particular fragment length may include measuring fluctuations of mcfNA fragments of a particular fragment length. In some embodiments, the fluctuations of mcfNA fragments of a particular fragment length may be analyzed over time. In some embodiments, the fluctuations of mcfNA fragments of a particular fragment length may be analyzed to assess a response to a treatment. In some embodiments, a fluctuation of mcfNA fragments of a particular fragment length are indicative of microbial replication. In some embodiments, the mcfNA fragments of a particular fragment length are about 55 nt. In some embodiments, about 55 nt comprises fragment lengths from about 45 nt to about 65 nt. In some embodiments, the mcfNA fragments of a particular fragment length are about 90 nt. In some embodiments, the mcfNA fragments of a particular fragment length are about 140 nt. In some embodiments, the mcfNA fragments of a particular fragment length are about 280 nt. In some embodiments, the mcfNA fragments of a particular fragment length are one or more of about 55 nt, about 90 nt, about 140 nt, and/or about 280 nt. The mcfNA fragments of one or more of fragment lengths may be modeled to a set of normal distributions. The mcfNA fragments of one or more of fragment lengths may have medians determined for the fragment length. The medians for the fragment length may be determined based on the normal distribution. For example, a normal distribution may be determined for mcfNA fragment lengths of about 55 nt and about 140 nt. In another example, a normal distribution may be determined for mcfNA fragment lengths of about 55 nt, about 90 nt, and about 140 nt. In these depicted examples, a median value at each length will also be determined. For example, in the first of the two examples the medians are determined for mcfNA fragment lengths of about 55 nt and about 140 nt; whereas in the second of the two examples the medians will be determined for mcfNA fragment lengths of about 55 nt, about 90 nt, and about 140 nt.

The mcfNA may be modeled using Gaussian distribution(s). A Gaussian distribution may be generated for the mcfNA fragments. Two Gaussian distribution may be generated for the mcfNA fragments. Three Gaussian distribution may be generated for the mcfNA fragments. The Gaussian distribution may comprise predetermined median lengths. The predetermined median lengths may be any about 55 nt, about 90 nt, about 140 nt, or any combination thereof. Active replication of a microbe may be detected when a Gaussian distribution with a median length of about 55 nt constitutes greater than about 5%, greater than about 10%, greater than about 15%, greater than about 20%, greater than about 25%, greater than about 30%, greater than about 35%, greater than about 40% m greater than about 50%, or greater than about 60% of all fragments of mcfNA in the sample. In some cases, the fragments are derived from the same microbe. Active replication of a microbe may be detected when a Gaussian distribution with a median length of about 55 nt constitutes greater than about 20% of all fragments of mcfNA in the sample.

In some embodiments, an active infection may be detected based on a greater percentage of a first mcfNA fragment of a particular fragment length as compared to all mcfNA fragments. In some embodiments, an active infection may be detected based on a greater percentage of a median of a first mcfNA fragment of a particular fragment length as compared to medians of all mcfNA fragments. For example, an active infection may be identified when the normal distribution with the median at the first fragment length constitutes greater than 20% of all fragments of microbial cell-free DNA in the sample. In some embodiments, the first fragment length is about 50 nt to about 300 nt. In some embodiments, the first fragment length is about 55 nt to about 280 nt. In some embodiments, the first fragment length is about 55 nt to about 140 nt. In some embodiments, the first fragment length is about 55 nt. In some embodiments, the median of the first fragment length is greater than about 5%, greater than about 10%, greater than about 15%, greater than about 20%, or greater than about 25% of all fragments of mcfNA in a sample. In some embodiments, the median of the first fragment length is less than about 5%, less than about 10%, less than about 15%, less than about 20%, less than about 25%, less than about 30%, less than about 35%, or less than about 40%, of all fragments of mcfNA in a sample.

A fragment length profile comprises one or more fragment length characteristics for a nucleic acid library or a subset of reads from within a nucleic acid library. A fragment length profile may comprise 1, 2, 3, 4, 5, 6, 7, 8, 9, 10, 11, 12, 13, 14, 15, 16, 17, 18, 19, 20, or more fragment length characteristics. A weighted value may be assigned to one or more fragment length characteristics in a fragment length profile such that one or more fragment length characteristics may have equal or different weights or values within the fragment length profile. Fragment length characteristics include, but are not limited to shape of the distribution, segment amplitude, peak shape, the fragment count ratio for two or more segments, the height of helical phasing peaks, fragment count ratio at two different fragment lengths, ratio of fragment counts within two different fragment length ranges, the fragment length range within a segment, the ratio of maximum amplitudes for two or more segments, position of a peak or peaks, and fragment length distribution within a subset of reads. It is intended that ratios “between 2 or more segments” encompasses, but is not limited to, two or more segments from one nucleic acid library, two or more segments from two or more nucleic acid libraries, two or more segments of the same peak shape, two or more segments of different peak shapes, two or more segments from similar or different nucleic acid library types and two or more segments from similar or different subsets of reads from a nucleic acid library.

Distribution types include, but are not limited to, a single peak shape, a multiple peak shape, exponential or exponential-like distributions, distributions inflated for long or short fragments, flat or uniform distributions, complex distribution shapes and combinations thereof. Complex distribution may include aspects of at least 2, at least 3, at least 4, at least 5, at least 6, at least 7, at least 8 or more peak shapes. A single peak shape may occur around any fragment length including but not limited to around the 50 or 55 nucleotide (nt) fragment length. Long fragments may include fragment lengths greater than about 50 nt, about 55 nt, about 60 nt, about 65 nt, about 70 nt, about 75 nt, about 80 nt, about 85 nt, about 90 nt, about 95 nt, about 100 nt, about 145 nt, about 150 nt, about 175 nt, about 200 nt, about 250 nt, about 280 nt, about 300 nt, about 350 nt and about 400 nt. Short fragments may include fragment lengths shorter than about 500 nt, about 400 nt, about 300 nt, about 280 nt, about 250 nt, about 200 nt, about 150 nt, about 145 nt, about 100 nt, about 95 nt, about 55 nt, about 50 nt, about 40 nt, about 35 nt, about 30 nt, about 25 nt, about 20 nt. Aspects of peak shape include but are not limited to the segment range, segment amplitude and the total number of reads within the segment, peak width, slope of the peak, derivative of the peak; aspects of peak shape may vary.

A single peak shape distribution may encompass a range of fragment lengths including but not limited to at least about 5 nt, at least about 10 nt, at least about 15 nt, at least about 20 nt, at least about 30 nt, at least about 35 nt, at least about 40 nt, at least about 45 nt, at least about 50 nt, at least about 55 nt, at least about 60 nt, at least about 65 nt, at least about 75 nt, at least about 85 nt, at least about 90 nt, at least about 95 nt, at least about 100 nt, at least about 110 nt, at least about 120 nt, at least about 130 nt, at least about 140 nt, at least about 145 nt, at least about 150 nt, at least about 175 nt, at least about 200 nt, at least about 225 nt, at least about 250 nt, at least about 280 nt, or at least about a 300 nt fragment length range within a segment. Fragment length range within a segment may vary. For example, the range of fragment length around a 55 nt single peak distribution includes but is not limited to fragment lengths from about 30 to 60 nt, about 35 to 60 nt, about 40 to 60 nt, and about 50 to 60 nt.

Segment amplitude encompasses the abundance or relative abundance of reads for a fragment length within a defined segment. In some aspects, the distribution amplitude may be the highest abundance or relative abundance within a defined fragment length range; distribution amplitude may also encompass the average highest abundance or relative abundance within a defined fragment length range. In some aspects of the application, a fragment length distribution or fragment length distribution profile is obtained for a subset of reads from a nucleic acid library. A subset of reads from a nucleic acid library is intended to encompass less than the full set of reads from a nucleic acid library. Subsets may reflect reads determined to be from a particular microbe type, from particular microbe species, host reads, maternal reads, fetal reads, organ donor reads, non-host reads, microbial cell-free nucleic acid reads, cell-free nucleic acid reads, microbial reads or any other group; alternatively, a subset of reads may reflect the full set of reads minus those from a particular microbe type, maternal read, fetal read or any other group. In some aspects of the application, a fragment length distribution is obtained for target nucleic acids.

### Microbes

As used herein, “microbe,” “microbial,” or “microorganism” refers to an organism, such as, for example, a microscopic or macroscopic organism, which may exist as a single cell or as a colony of cells, capsids, spores, filaments, or multicellular organisms. Microbes include all unicellular organisms and some multicellular organisms, such as, for example, those from archaea, bacteria, protozoa, nematodes, viruses and eukaryotes. Microbes are often pathogens responsible for disease, but may also exist in a non-pathogenic, symbiotic relationship with a host, such as a human. A “commensal microorganism” is intended to include microbes that exist in a non-pathogenic, symbiotic relationship with a host. A host organism may harbor multiple types of non-host organisms simultaneously. In co-infection a host organism harbors multiple types of non-host organisms. The multiple types of non-host organisms may include one or more pathogens, one or more commensal microorganisms, or at least one pathogen and at least one commensal microorganism. The methods of the current application may be used to distinguish between closely related microorganisms, distinguish between microbes present as a pathogen, a commensal microorganism, or as incidental but clinically unimportant microbes.

Microbes or pathogens may include archaea, bacteria, yeast, fungi, molds, protozoans, nematodes, eukaryotes, and/or viruses. Microbes or pathogens may also include DNA viruses, RNA viruses, culturable bacteria, additional fastidious and unculturable bacteria, mycobacteria, and eukaryotic pathogens (See, Bennett J. E., D., R., Blaser, M. J. Mandell, Douglas, and Bennett's Principles and Practice of Infectious Diseases; Saunders, Philadelphia, Pa., 2014; and Netter's Infectious Disease, 1st Edition, edited by Elaine C. Jong, M D and Dennis L. Stevens, M D, PhD (2015)). Microbes or pathogens may also include any of the microbes set forth in https://www.ncbi.nlm.nih.gov/genome/microbes/or https://www.ncbi.nlm.nih.gov/biosample/.

In some embodiments, the microbe is associated with a disease or infection. In some embodiments, the microbe is a bacterium. In some embodiments, the microbe is a virus. In some embodiments, the virus is a DNA virus. In some embodiments, the virus is a double-stranded DNA virus. In some embodiments, the virus is an RNA virus. In some embodiments, the virus is an enveloped virus. In some embodiments, the virus is Epstein-Barr virus (EBV). In some embodiments, the virus is herpes simplex virus (HMV). In some embodiments, the virus is human T-lymphotropic virus (HTLV). In some embodiments, the virus is cytomegalovirus (CMV). In some embodiments, the CMV is porcine CMV (pCMV). In some embodiments, the CMV is bovine CMV (bCMV). In some embodiments, the virus is in the order Herpesvirales. In some embodiments, the microbe is a fungus. In some embodiments, the microbe is associated with a transplant. In some embodiments, the microbe is associated with a transplanted organ. In some embodiments, the microbe is associated with a transplanted graft. In some embodiments, the microbe is associated with a xenotransplant. In some embodiments, the microbe is associated with a xenotransplanted organ. In some embodiments, the microbe is associated with a xenograft. In some embodiments, the microbe is associated with an allotransplant. In some embodiments, the microbe is associated with an allotransplanted organ. In some embodiments, the microbe is associated with an allograft.

Examples of microbes are one or more of the species or strains from one or more of the following genera: Coniosporium, Hantavirus, Talaromyces, Machlomovirus, Betatetravirus, Raoultella, Aeromonas, Ephemerovirus, Empedobacter, Loa, Macluravirus, Stenotrophomonas, Alfamovirus, Rosavirus, Emmonsia, Aggregatibacter, Orthopneumovirus, Weeksella, Nairovirus, Salivirus, Weissella, Mosavirus, Gammapartitivirus, Strongyloides, Passerivirus, Erysipelatoclostridium, Bacillarnavirus, Iotatorquevirus, Taenia, Trypanosoma, Olsenella, Cladosporium, Rhizobium, Prevotella, Leclercia, Paracoccus, Ilarvirus, Lagovirus, Rasamsonia, Plasmodium, Acremonium, Chlamydia, Clonorchis, Vibrio, Bartonella, Nakazawaea, Franconibacter, Anisakis, Norovirus, Nocardia, Solobacterium, Parechovirus, Avenavirus, Orthohepevirus, Aphthovirus, Hepandensovirus, Microbacterium, Lichtheimia, Lomentospora, Achromobacter, Ipomovirus, Tsukamurella, Elizabethkingia, Hepevirus, Seadomavirus, Altemaria, Trueperella, Gammatorquevirus, Bifidobacterium, Chrysosporium, Thogotovirus, Curtovirus, Deltatorquevirus, Balamuthia, Mastrevirus, Bdellomicrovirus, Mupapillomavirus, Pseudozyma, Wickerhamiella, Aquamavirus, Alloscardovia, Thielavia, Idaeovirus, Henipavirus, Coxiella, Haemophilus, Gammacoronavirus, Negevirus, Brevibacterium, Peptoniphilus, Alphacarmotetravirus, Nosema, Trichovirus, Arenavirus, Thermomyces, Necator, Waikavirus, Blosnavirus, Jonesia, Tetraparvovirus, Emaravirus, Plectrovirus, Sclerodarnavirus, Toxocara, Umbravirus, Burkholderia, Chromobacterium, Paracoccidioides, Brugia, Eragrovirus, Macrococcus, Absidia, Colletotrichum, Inovirus, Phycomyces, Wickerhamomyces, Acidaminococcus, Moraxella, Rothia, Phlebovirus, Slackia, Purpureocillium, Betapapillomavirus, Tupavirus, Cryspovirus, Saksenaea, Erysipelothrix, Kobuvirus, Mimoreovirus, Echinococcus, Mannheimia, Bergeyella, Cyclospora, Xylanimonas, Leptospira, Finegoldia, Curvularia, Cryptosporidium, Babuvirus, Pecluvirus, Lambdatorquevirus, Pythium, Carlavirus, Entomobimavirus, Kocuria, Anaplasma, Ampelovirus, Avihepatovirus, Nepovirus, Rhodococcus, Bordetella, Mischivirus, Scedosporium, Gardnerella, Maculavirus, Trichoderma, Aveparvovirus, Salmonella, Avastrovirus, Copiparvovirus, Trachipleistophora, Clostridioides, Nanovirus, Siccibacter, Leptotrichia, Citrivirus, Odoribacter, Sanguibacter, Novirhabdovirus, Acremonium, Hafnia, Chaetomium, Tenuivirus, Yokenella, Rubulavirus, Varicellovirus, Alphamesonivirus, Sicinivirus, Leuconostoc, Microvirus, Gallantivirus, Morbillivirus, Lolavirus, Pantoea, Hepatovirus, Nupapillomavirus, Metschnikowia, Barnavirus, Kytococcus, Tritimovirus, Tannerella, Respirovirus, Pneumocystis, Dirofilaria, Pediococcus, Lactococcus, Blastomyces, Dianthovirus, Actinobacillus, Teschovirus, Oscivirus, Begomovirus, Potyvirus, Byssochlamys, Alphacoronavirus, Molluscipoxvirus, Lymphocryptovirus, Sapelovirus, Parabacteroides, Pyrenochaeta, Listeria, Senecavirus, Brevidensovirus, Potexvirus, Parvimonas, Flavivirus, Recovirus, Toxoplasma, Yatapoxvirus, Opisthorchis, Trichuris, Cyphellophora, Morganella, Perhabdovirus, Micrococcus, Pequenovirus, Mastadenovirus, Anaeroglobus, Tropheryma, Dolosigranulum, Wolbachia, Lelliottia, Mycoplasma, Tobravirus, Shewanella, Paeniclostridium, Erythroparvovirus, Sutterella, Sporopachydermia, Narnavirus, Nyavirus, Francisella, Arthroderma, Epsilontorquevirus, Sigmavirus, Amdoparvovirus, Actinomyces, Alphapermutotetravirus, Cardiobacterium, Influenzavirus C, Orthopoxvirus, Poacevirus, Phialophora, Lactobacillus, Polyomavirus, Debaryomyces, Foveavirus, Bymovirus, Mycoflexivirus, Grimontia, Mucor, Rhytidhysteron, Quadrivirus, Thermoascus, Aureusvirus, Trichosporon, Myceliophthora, Dermacoccus, Dysgonomonas, Pseudoramibacter, Becurtovirus, Gordonia, Sapovirus, Orthobunyavirus, Spiromicrovirus, Pomovirus, Exophiala, Sneathia, Helicobacter, Photorhabdus, Mogibacterium, Betapartitivirus, Avibirnavirus, Ambidensovirus, Oleavirus, Orientia, Deltacoronavirus, Anulavirus, Trichomonasvirus, Budvicia, Geotrichum, Enamovirus, Lachnoclostridium, Schistosoma, Paecilomyces, Panicovirus, Rhizoctonia, Brevibacillus, Beauveria, Pestivirus, Tombusvirus, Cilevirus, Cokeromyces, Peptostreptococcus, Phanerochaete, Proteus, Idnoreovirus, Aspergillus, Pasteurella, Malassezia, Hanseniaspora, Endornavirus, Azospirillum, Velarivirus, Cystovirus, Avisivirus, Bacteroides, Picobimavirus, Myroides, Circovirus, Arterivirus, Aquaparamyxovirus, Onchocerca, Cosavirus, Kluyveromyces, Fijivirus, Candida, Hepacivirus, Dermabacter, Ourmiavirus, Allexivirus, Enterobacter, Acidovorax, Bracorhabdovirus, Carmovirus, Pluralibacter, Coltivirus, Fonsecaea, Streptobacillus, Corynebacterium, Macrophomina, Marburgvirus, Comovirus, Fabavirus, Alphanodavirus, Cellulomonas, Enterobius, Catabacter, Moellerella, Nakaseomyces, Cucumovirus, Valsa, Deltapartitivirus, Plesiomonas, Pseudomonas, Torovirus, Cuevavirus, Hypovirus, Trichomonas, Influenzavirus D, Giardiavirus, Crinivirus, Tepovirus, Sakobuvirus, Cyberlindnera, Paenalcaligenes, Bafinivirus, Rymovirus, Pegivirus, Yarrowia, Treponema, Borreliella, Rubivirus, Aureobasidium, Angiostrongylus, Filobasidium, Photobacterium, Rhizopus, Orthoreovirus, Ustilago, Simplexvirus, Aquareovirus, Protoparvovirus, Propionibacterium, Sprivivirus, Hunnivirus, Apophysomyces, Meyerozyma, Alphapapillomavirus, Brucella, Gallivirus, Dinovernavirus, Anaerobiospirillum, Eubacterium, Tatlockia, Terri sporobacter, Quaranjavirus, Sobemovirus, Dicipivirus, Arcanobacterium, Macanavirus, Atopobium, Vesivirus, Lodderomyces, Dinornavirus, Betatorquevirus, Kerstersia, Aparavirus, Neisseria, Agrobacterium, Edwardsiella, Labyrnavirus, Totivirus, Actinomadura, Tobamovirus, Influenzavirus B, Mandarivirus, Anaerococcus, Kunsagivirus, Naegleria, Campylobacter, Veillonella, Yamadazyma, Filobasidiella, Oerskovia, Penicillium, Anncaliia, Leptosphaeria, Pneumovirus, Psychrobacter, Isavirus, Granulicatella, Torradovirus, Cladophialophora, Influenzavirus A, Ophiostoma, Aerococcus, Ureaplasma, Etatorquevirus, Bocaparvovirus, Megasphaera, Reptarenavirus, Comamonas, Capnocytophaga, Alphatorquevirus, Syncephalastrum, Wallemia, Betacoronavirus, Hyphopichia, Nocardiopsis, Legionella, Trichinella, Paraburkholderia, Mammarenavirus, Echinostoma, Sphingobacterium, Enterovirus, Methanobrevibacter, Ochroconis, Cheravirus, Pasivirus, Enterococcus, Mycoreovirus, Tospovirus, Betanodavirus, Phytoreovirus, Enterocytozoon, Ferlavirus, Stemphylium, Filifactor, Leishmaniavirus, Gemella, Bromovirus, Alloiococcus, Cunninghamella, Cronobacter, Oribacterium, Orbivirus, Chrysovirus, Cripavirus, Tatumella, Pandoraea, Ogataea, Dracunculus, Volvariella, Iflavirus, Benyvirus, Rhadinovirus, Histoplasma, Rahnella, Morococcus, Verticillium, Janibacter, Gyrovirus, Alphapartitivirus, Mycobacterium, Roseomonas, Varicosavirus, Chryseobacterium, Parapoxvirus, Rhizomucor, Aureimonas, Levivirus, Leishmania, Luteovirus, Cypovirus, Ochrobactrum, Microsporum, Piscihepevirus, Ceratocystis, Sporothrix, Vesiculovirus, Cupriavidus, Cryptococcus, Metapneumovirus, Alphanecrovirus, Eikenella, Brevundimonas, Escherichia, Leifsonia, Schizophyllum, Granulibacter, Gordonibacter, Lachancea, Madurella, Ophiovirus, Phellinus, Nebovirus, Acanthamoeba, Fusobacterium, Pichia, Verruconis, Ehrlichia, Tibrovirus, Higrevirus, Wohlfahrtiimonas, Rhinocladiella, Neorickettsia, Sadwavirus, Roseobacter, Sequivirus, Pannonibacter, Rotavirus, Turicella, Cardiovirus, Propionimicrobium, Furovirus, Naumovozyma, Closterovirus, Fluoribacter, Zeavirus, Clavispora, Megrivirus, Gammapapillomavirus, Rickettsia, Polemovirus, Corynespora, Encephalitozoon, Shimwellia, Fusarium, Yersinia, Capronia, Delftia, Victorivirus, Marafivirus, Kluyvera, Iteradensovirus, Isoptericola, Vitivirus, Roseolovirus, Conidiobolus, Abiotrophia, Babesia, Phoma, Sanguibacteroides, Staphylococcus, Rhodotorula, Zetatorquevirus, Hymenolepis, Fasciola, Cytorhabdovirus, Cardoreovirus, Memnoniella, Trichophyton, Mitovirus, Phaeoacremonium, Providencia, Lysinibacillus, Giardia, Oligella, Streptomyces, Paraclostridium, Ralstonia, Coccidioides, Brambyvirus, Biatriospora, Allolevivirus, Acinetobacter, Starmerella, Omegatetravirus, Porphyromonas, Avulavirus, Streptococcus, Arcobacter, Topocuvirus, Mamastrovirus, Ancylostoma, Bornavirus, Capillovirus, Alphavirus, Tymovirus, Nucleorhabdovirus, Diaporthe, Chlamydiamicrovirus, Tumeurtovirus, Saccharomyces, Riemerella, Betanecrovirus, Clostridium, Mobiluncus, Cercospora, Marnavirus, Mortierella, Aquabimavirus, Xanthomonas, Dependoparvovirus, Ebolavirus, Neofusicoccum, Borrelia, Leminorella, Klebsiella, Blastocystis, Alcaligenes, Citrobacter, Eggerthella, Cedecea, Serratia, Penstyldensovirus, Bacillus, Laribacter, Wuchereria, Hordeivirus, Cytomegalovirus, Actinomucor, Ascaris, Shigella, Vittaforma, Torulaspora, Kingella, Oryzavirus, Polerovirus, Tremovirus, Erbovirus, Entamoeba, Lyssavirus, Paenibacillus, Facklamia, Kappatorquevirus, Metarhizium, Stachybotrys, Okavirus, Botrexvirus, Thetatorquevirus, and Basidiobolus.

### Treatments

The present disclosure also provides methods for individualized treatment for an infected subject or a subject who is susceptible or at risk for infections (e.g., immunosuppressed, immunocompromised, transplant patient). Individualized treatment can include predicting if an infection will progress to an invasive disease stage, monitoring the efficacy of a therapy in a subject, modifying a therapeutic regimen depending on the subject's response to the therapy, or determining the pathogen's resistance to a particular therapeutic. In some cases, the methods can be used to detect, diagnose, predict, or prognose the pathogen's resistance to a particular therapeutic.

In some cases, samples may be collected serially at various times before or during the course of the infection to determine the pathogen's and subject's response to a treatment, and may provide a regimen that is individually tailored. In some cases, the serially-collected samples are compared to each other to determine whether the infection is improving or worsening in the subject.

The treatment may involve administering a drug or other therapy to reduce or eliminate the colonization or invasive disease associated with the infection. In some cases, the subject may be treated prophylactically to prevent the development of an infection. Any medical procedure or treatment including administration of a drug can be used to improve or reduce the symptoms of an infection. The treatment may comprise antimicrobials. The antimicrobials may be an antibiotic. The antimicrobial may be an antibacterial. The antimicrobial may be an antifungal. The antimicrobial may be an antiviral. The treatment may comprise an antibiotic. The treatment may comprise an antibacterial. The treatment may comprise an antifungal. The treatment may comprise an antiviral. The treatment may be administered to the subject. The treatment may be administered to a human subject. In some embodiments, coverage of mcfDNA at or near the origin of replication is monitored for response to treatment. In some embodiments, the subject is administered an immunosuppressant drug. In some embodiments, the immunosuppressant drug is administered to a transplant recipient. In some embodiments, the transplant recipient is showing signs of transplant rejection. In some embodiments, the immunosuppressant is used to treat transplant rejection.

In some embodiments, when an actively replicating microbe is detected, an antimicrobial treatment (e.g., antiviral, antibiotic) may be administered to a transplant recipient. In some cases, when an actively replicating microbe is detected, the dose of the antimicrobial treatment may need to be adjusted, e.g., the dose may need to be increased.

Some nonlimiting exemplary drugs that can be used are antibiotics (such as ampicillin, sulbactam, penicillin, vancomycin, gentamycin, aminoglycoside, clindamycin, cephalosporin, metronidazole, timentin, ticarcillin, clavulanic acid, cefoxitin), antiretroviral drugs (e.g., highly active antiretroviral therapy (HAART), reverse transcriptase inhibitors, nucleoside/nucleotide reverse transcriptase inhibitors (NRTIs), Non-nucleoside RT inhibitors, and/or protease inhibitors), or immunoglobulins.

The present disclosure also provides methods of adjusting a therapeutic regimen. For example, the subject may have been administered a drug to treat the infection. The methods provided herein may be used to track or monitor the efficacy of the drug treatment. In some cases, the therapeutic regimen may be adjusted, depending on upward or downward course of the infection. For example, if the methods provided herein indicate that an infection is not improving with drug treatment, the therapeutic regimen may be adjusted by changing the type of drug or treatment, discontinuing the use of the drug, continuing the use of the drug, increasing the dose of the drug, or adding a new drug or treatment to the subject's therapeutic regimen.

In some cases, the therapeutic regimen may involve a particular procedure. For example, in some cases, the methods may indicate a need for a surgical procedure or an invasive diagnostic procedure such as performing a biopsy to determine if an organ is infected. Likewise, if the methods indicate than an infection is improving or resolved by a therapeutic intervention, then adjusting a therapeutic regimen may involve reducing or discontinuing the treatment. In other cases, no therapeutic regimen may be given instead “watchful waiting” or “watch and wait” approach may be used to see if the infection clears up without any additional medical intervention.

The methods of the disclosure may comprise detection of a pathogen in a subject. In some cases, more than one pathogen is detected in a subject. In some cases, the method can comprise using whole-genome sequencing of the sample. In some cases, the method can comprise using targeted sequencing of the sample, where specific primers are used to detect a particular pathogen of interest. Often, a pathogen can have a suggested treatment cycle.

An antimicrobial agent may be used to treat a subject. The antimicrobial agent may be any antimicrobial agent known to those of ordinary skill in the art. For example, the antimicrobial agent may be an antibiotic, an antiviral agent, or an antifungal agent. An “antibiotic” is defined herein to refer to a compound or agent that can prevent or reduce the growth and reproduction of a bacterium or kill a bacterium. Some antibiotics kill bacteria, whereas others prevent or inhibit their growth. Antibiotics are applied in the treatment of subjects with infections, such as bloodstream infections. They are administered via any of a variety of routes, such as through oral, intravenous, subcutaneous, or intramuscular routes. Examples of antibiotics include penicillin, cephalosporins, vancomycin, minocycline, and rifampin. In certain embodiments, the antibiotic is a tetracycline or a macrocyclic antibiotic or a combination thereof. The tetracycline can be any tetracycline known to those of ordinary skill in the art, such as minocycline. Non-limiting examples of macrocyclic antibiotics include rifampin, rifampicin, or a combination thereof.

As used herein, the term “antifungal agent” is defined as a compound having either a fungicidal or fungistatic effect upon fungi contacted by the compound. As used herein, the term “fungicidal” is defined to mean having a destructive killing action upon fungi. As used herein, the term “fungistatic” is defined to mean having an inhibiting action upon the growth of fungi. Some exemplary classes of antifungal agents include imidazoles or triazoles such as clotrimazole, miconazole, ketoconazole, econazole, butoconazole, omoconazole, oxiconazole, terconazole, itraconazole, fluconazole, voriconazole, posaconazole, ravuconazole or flutrimazole; the polyene antifungals such as amphotericin B, liposomal amphoterecin B, natamycin, nystatin and nystatin lipid formulations; the cell wall active cyclic lipopeptide antifungals, including the echinocandins such as caspofungin, micafungin, anidulfungin, cilofungin; LY121019; LY303366; the allylamine group of antifungals such as terbinafine. Yet other non-limiting examples of antifungal agents include naftifine, tolnaftate, mediocidin, candicidin, trichomycin, hamycin, aurefungin, ascosin, ayfattin, azacolutin, trichomycin, levorin, heptamycin, candimycin, griseofulvin, BF-796, MTCH 24, BTG-137586, pradimicins (MNS 18184), benanomicin; ambisome; nikkomycin Z; flucytosine, or perimycin.

Additional examples of antibiotics include, without limitation, beta-lactam antibiotics, sulfonamides and quinolones. Examples of beta-lactam antibiotics include, but are not limited to, penicillin derivatives, cephalosporins, penems, monobactams, carbapenems, beta-lactamase inhibitors and combinations thereof. Examples of penicillin derivatives include, but are not limited to, aminopenicillins (e.g. amoxicillin, ampicillin, and epicillin); carboxypenicillins (e.g. carbenicillin, ticarcillin, and temocillin); ureidopenicillins (e.g. azlocillin, piperacillin and mezlocillin); mecillinam, sulbenicillin, benzathine penicillin, penicillin G (benzylpenicillin), penicillin V (phenoxymethylpenicillin), penicillin O (allylmercaptomethylpenicillinic), procaine penicillin, oxacillin, methicillin, nafcillin, cloxacillin, dicloxacillin, flucloxacillin, pivampicillin, hetacillin, becampicillin, metampicillin, talampicillin, co-amoxiclav (amoxicillin plus clavulanic acid), and piperacillion. Examples of cephalosporins include, but are not limited to, cephalexin, cephalothin, cefazolin, cefaclor, cefuroxime, cefamandole, cefotetan, cefoxitin, ceforanide, ceftriaxone, cefotaxime, cefpodoxime proxetil, ceftazidime, cefepime, cefoperazone, ceftizoxime, cefixime and cefpirome. Examples of penems include, without limitation, faropenem. Examples of monobactams include, without limitation, aztreonam and tigemonam. Examples of carbapenems include, but are not limited to, biapenem, doripenem, ertapenem, imipenem, meropenem, and panipenem. Examples of beta-lactamase inhibitors include, but are not limited to, tazobactam ([2S-(2alpha,3beta,5alpha)]-3-Methyl-7-oxo-3-(1H-1,2,3-triazol-1-ylmethyl)-4-thia-1-azabicyclo[3.2.0]heptane-2-carboxylic acid 4,4-dioxide sodium salt), sulbactam (2S,5R)-3,3-dimethyl-7-oxo-4-thia-1-azabicyclo[3.2.0]heptane-2-carboxylic acid 4,4-dioxide sodium), and clavulanic acid ((2R,5R,Z)-3-(2-hydroxyethylidene)-7-oxo-4-oxa-1-aza-bicyclo[3.2.0]heptane-2-carboxylic acid). Other examples of antibiotics include, without limitation, [(N-benzyloxycarbonylamino)methyl]-phosphonic acid mono-(4-nitrophenyl) ester sodium salt, [(N-benzyloxycarbonylamino)methyl]-phosphonic acid mono-(3-pyridinyl) ester sodium salt, sulfanilamide (4-aminobenzenesulfonamide), sulfasalazine (6-oxo-3-(2-[4-(N-pyridin-2-ylsulfamoyl)phenyl]hydrazono)cyclohexa-1,4-dienecarboxylic acid), 1-cyclopropyl-6-fluoro-4-oxo-7-piperazin-1-yl-quinoline-3-carboxylic acid, nalidixic acid (1-ethyl-7-methyl-4-oxo-[1,8]naphthyridine-3-carboxylic acid),

Examples of sulfonamides include, without limitation, sulfaisodimidine, sulfanilamide, sulfadiazine, sulfisoxazole, sulfamethoxazole, sulfadimethoxine, sulfamethoxypyridazine, sulfacetamide, sulfadoxine, acetazolamide, bumetanide, chlorthalidone, clopamide, furosemide, hydrochlorothiazide, indapamide, mefruside, metolazone, xipamide, dichlorphenamide, dorzolamide, acetazolamide, ethoxzolamide, sultiame, zonisamide, mafenide, celecoxib, darunavir, probenecid, sulfasalazine, and sumatriptan.

Examples of quinolones include, without limitation, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, garenoxacin, ecinofloxacin, delafloxacin and nalidixic acid.

Additional examples of antifungals include, without limitation, polyene antifungals (e.g. natamycin, rimocidin, filipin, nystatin, amphotericin B, candicin), imidazole antifungals (e.g. miconazole, ketoconazloe, clotrimazole, econazole, bifonazole, butoconazole, fenticonazole, isoconazole, oxiconazole, sertaconazole, sulconazole, and tioconazole), triazoles antifungals (e.g. fluconazole, itraconazole, isavuconazole, ravuconazole, posaconazole, voriconazole, and terconazole), thiazole antifungals (e.g. abafungin), allyamines (e.g. terbinafine, amorolfine, naftifine and butenafine), echinocandins (e.g. anidulafungin, caspofungin and micafungin) and other antifungals such as benzoic acid, ciclopirox, tolnaftate, undecylenic acid, flucytosine, griseofulvin, haloprogin.

Examples of antiprotozoals include, without limitation, elomithine, furazolidone, melarsoprol, metronidazole, ornidazole, paromomycin sulfate, pentamidine, pyrimethamine, and tinidazole.

As used herein, the term “antiviral agent” is defined as a compound that can either kill viral agents or one that stops the replication of viruses upon contact by the compound. Non-limiting examples of antiviral agents include cidofovir, amantadine, rifampicin, zanamivir, oseltamivir, rimantadine, acyclovir, gancyclovir, pencyclovir, famciclovir, foscamet, ribavirin, or valcyclovir. In some embodiments the antimicrobial agent is an innate immune peptide or proteins. Some exemplary classes of innate peptides or proteins are transferrins, lactoferrins, defensins, phospholipases, lysozyme, cathelicidins, serprocidins, bactericidal permeability increasing proteins, amphipathic alpha helical peptides, and other synthetic antimicrobial proteins.

Additional examples of antivirals include, but are not limited to an abacavir, an acyclovir (aciclovir), an adefovir, an amantadine, an ampligen, an amprenavir (agenerase), an umifenovir (arbidol), an atazanavir, an atripla (efavirenz/emtricitabine/tenofovir), a baloxavir marboxil (xofluza), a biktarvy (bictegravir/emtricitabine/tenofovir alafenamide), a boceprevir, a bulevirtide, a cidofovir, a cobicistat (tybost), a combivir (lamivudine/zidovudine), a daclatasvir (daklinza), a darunavir, a delavirdine, a descovy (emtricitabine/tenofovir alafenamide), a didanosine, a docosanol, a dolutegravir, a doravirine (pifeltro), an edoxudine, an efavirenz, an elvitegravir, an emtricitabine, an enfuvirtide, an ensitrelvir, an entecavir, an etravirine (intelence), a famciclovir, a fomivirsen, a fosamprenavir, a foscarnet, a ganciclovir (cytovene), an ibacitabine, an ibalizumab (trogarzo), an idoxuridine, an imiquimod, an inosine pranobex, an indinavir, a lamivudine, a letermovir (prevymis), a lopinavir, a loviride, a maraviroc, a methisazone, a moroxydine, a nelfinavir, a nirmatrelvir/ritonavir (paxlovid), a nevirapine, a nitazoxanide, a norvir, an oseltamivir (tamiflu), a penciclovir, a peramivir, a penciclovir, a peramivir (rapivab), a pleconaril, a podophyllotoxin, a raltegravir, a remdesivir, a ribavirin, a rilpivirine (edurant), a rilpivirine, a rimantadine, a ritonavir, a saquinavir, a simeprevir (olysio), a sofosbuvir, a stavudine, a taribavirin (viramidine), a telaprevir, a telbivudine (tyzeka), a tenofovir alafenamide, a tenofovir disoproxil, a tipranavir, a trifluridine, a trizivir, a tromantadine, a truvada, an umifenovir, a valaciclovir (valtrex), a valganciclovir (valcyte), a vicriviroc, a vidarabine, a zalcitabine, a zanamivir (relenza), a zidovudine.

Any of the antimicrobials disclosed herein include a stereoisomer of any of these, a salt of any of these, or a combination of any of these.

### Systems

Disclosed herein are systems configured to perform any of the methods disclosed herein. The systems disclosed herein comprise computer readable media. A system can include an apparatus for detection and/or computer control systems with machine-executable instructions to implement the methods. In some embodiments, the computer control systems are further programmed for conducting genetic analysis.

Detection systems that can be used with the methods of the present disclosure can include but are not limited to sequencing, PCR, digital PCR, ddPCR, quantitative PCR (e.g., real-time PCR) or by a microfluidic device, microarray, or the like.

### Sequencing

A system can include a nucleic acid sequencer (e.g., DNA sequencer, RNA sequencer) for generating DNA or RNA sequence information. The system may further include a computer comprising software that performs bioinformatics analysis on the DNA or RNA sequence information. Bioinformatics analysis can include, without limitation, assembling sequence data, detecting and quantifying genetic variants in a sample, including germline variants and somatic cell variants (e.g., a genetic variation associated with infection).

Sequencing data may be used to determine genetic sequence information, ploidy states, the identity of one or more genetic variants, as well as a quantitative measure of the variants, including relative and absolute relative measures. In some cases, sequencing of the genome involves whole genome sequencing or partial genome sequencing. The sequencing may be unbiased and may involve sequencing all or substantially all (e.g., greater than 70%, 80%, 90%) of the nucleic acids in a sample. Sequencing of the genome can be selective, e.g., directed to portions of the genome of interest. For example, many genes (and mutant forms of these genes) are known to be associated with various cancers. Sequencing of select genes, or portions of genes may suffice for the analysis desired. Polynucleotides mapping to specific loci in the genome that are the subject of interest can be isolated for sequencing by, for example, sequence capture or site-specific amplification.

### Digital PCR

In some applications, a system can include an apparatus for digital PCR or droplet based digital PCR. A digital PCR assay can be multiplex, such that two or more different analytes or nucleic acid forms are detected within a single partition (e.g., reaction mixture). Amplification of the analytes can be distinguished by utilizing analyte-specific probes labeled with different fluorophores or dyes. A digital PCR machine may comprise a detector that can distinguishably measure the fluorescence of the different labels, and thereby detect different analytes.

Measurements can include the determination of microbial read coverage, copy number, the status of a single nucleotide polymorphisms, deletions, duplications, translocations, and/or inversions, which can be the source of disease, susceptibility to disease and/or responsiveness to particular therapeutic treatment.

### Real-Time PCR Methodologies

In some applications a system can include an apparatus for real-time PCR (or quantitative PCR (qPCR). A real-time polymerase chain reaction can be configured for multiplexing by using emission differences of between two or more fluorescent probes or dyes.

### Microarray

In some applications, a system can include an apparatus for microarray detection. Microarray maybe desirable in cases where the methods are being applied in a targeted fashion. In some applications, arrays may be subdivided with a gasket into subarrays.

A microarray is device generally contains short single-stranded oligonucleotide probes (e.g., 25- to 70-bp in length) attached to a solid substrate. The probes can be designed to have sequences complementary to the targets of interest. Targeted oligos can be added the microarray by spotting, spraying, or synthesized in situ through a series of photocatalyzed reactions.

### Microfluidic Devices

In some applications, a system can include a microfluidic apparatus for carrying put the methods of the disclosure. A microfluidic device used with the methods of the disclosure can be configured to perform various amplification assays including PCR, qPCR, or RT-PCR. In some applications, the microfluidic device can also be configured to integrate pre-PCR or post-PCR assays.

### Computer Control Systems

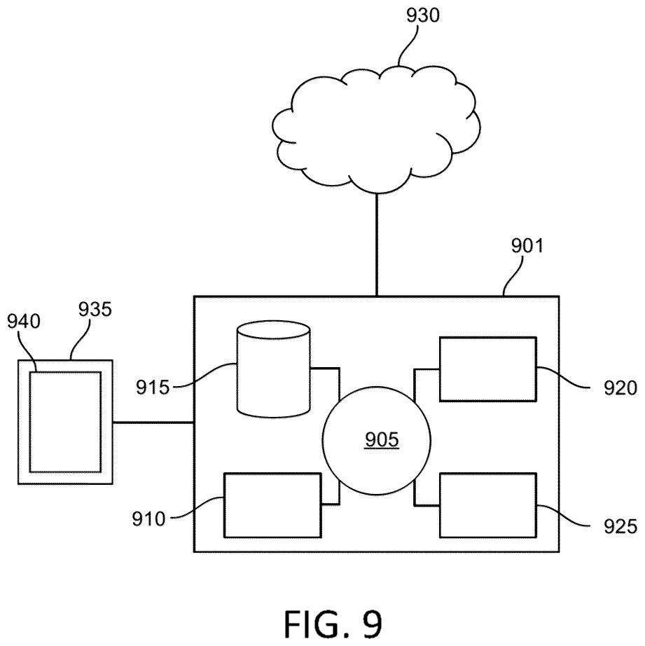

The disclosure also provides computer control systems programmed to implement the methods of the disclosure. FIG. 9 shows a computer system 901 that is programmed or otherwise configured to implement methods of the present disclosure.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, an output of a report, which may include a diagnosis of a subject or a therapeutic intervention for the subject. Examples of UI's include, without limitation, a graphical user interface (GUI) and web-based user interface. The analysis can be provided as a report. The report may be provided to a subject, to a health care professional, a lab-worker, or other individual.

Methods and systems of the present disclosure can be implemented by way of one or more algorithms. An algorithm can be implemented by way of software upon execution by the central processing unit 905. The algorithm can, for example, facilitate the enrichment, sequencing and/or detection of pathogen or other target nucleic acids.

Information about a patient or subject can be entered into a computer system, for example, patient background, patient medical history, or medical scans. The computer system can be used to analyze results from a method described herein, report results to a patient or doctor, or come up with a treatment plan.

### EXAMPLES

### Example 1: Microbial Fragmentomics Indicates Post-Xenotransplant Viral Replication Background to Experiment

In early 2022, likely the first pig-to-human cardiac xenotransplantation was performed on a 57-year-old man with severe heart failure. The patient survived for 60 days post-transplantation, and throughout this period, the circulating levels of porcine cell-free DNA (cfDNA) and microbial cell-free DNA (mcfDNA) were surveilled. On post-transplant day 20, mcfDNA from porcine cytomegalovirus (pCMV) was observed in the patient's plasma. On day 43, the patient developed signs of infection that abated in apparent response to a change from ganciclovir to cidofovir directed at pCMV. From day 49, the patient's condition deteriorated as the left and right ventricular wall thickness increased concomitant with a reduction in the size of the left ventricular chamber. Indeed, from day 49 until the withdrawal of support on day 60, the patient experienced progressive cardiac failure secondary to unexplained irreversible xenograft injury.

The specific cause of the transplant injury was poorly understood and there may several possible explanations for the injury, including rejection and/or infections (e.g., bacterial and/or viral). Increases in viral concentration, however, do not necessarily indicate active replication or to what extent the presence of the virus impacts outcome. The case of xenograft transplant injury demonstrates the need for improved methods for transplant analysis. The present disclosure provides methods that can identify causes of transplant injury. The methods of the disclosure may provide a clearer picture of the role played by an infection, if any, in a transplant injury.

Infections in solid organ transplant recipients are a considerable cause of mortality (13-16% in kidney and heart transplant recipients, up to 21% in lung, and approximately 50% within the first year in liver), and previous work was reported as showing that pCMV infection in orthotopic porcine cardiac xenotransplantation into baboons correlated strongly with reduced survival. Obtaining a deeper knowledge of pCMV activity, replication, and growth rates could help determine whether pCMV is latent or active throughout a patient's treatment, and may further assist in elucidating whether and to what extent such viral activity will impact a course of the disease and ultimately the outcome.

Studying the coverage and fragmentation patterns of cfDNA can be a powerful tool for a growing number of clinical applications. In bacteria, a quantitative proxy for microbial growth rates can be bioinformatically inferred by comparing sequencing coverage at the origin and terminus of replication. High rates of replication in pathogenic bacteria may precede the development of necrotizing enterocolitis in-vivo. Microbial mcfDNA from urine may be used to distinguish pathogenic microbes in the urinary tract from commensals. A distinct population of short cfDNAs may be released by microbial cells during proliferation. A study of HHV-6 mcfDNA may show nucleosome-sized viral cfDNA fragments that appear indicative of a latent virus, integrated into the host's genome; however, shorter viral cfDNAs may indicate an active, replicating virus.

Here microbial fragmentomics approaches to study the pCMV-derived mcfDNA through the 60-day time course of the porcine-to-human cardiac xenotransplantation patient were expanded. An evident increase in pCMV concentration, the non-uniform mcfDNA were observed surrounding the origin of replication, and the emergence of shorter mcfDNA fragments over time, together suggest that pCMV was likely to have been actively replicating through the later stages of hospitalization. When applying microbial fragmentomics methods to the question of microbial replication more broadly, the viral replication signal was found to be evident from mcfDNA coverage in a large cohort of 391 case-patients with either human herpesvirus 6B, herpesvirus 7, or CMV detections.

Plasma mcfDNA sequencing was performed in a Clinical Laboratory Improvement Amendments/College of American Pathologists-accredited laboratory (Karius Inc, Redwood City, California), referred to herein as the Karius Test (KT), to provide unbiased testing for human pathogens. The assay was designed to report the absolute concentration of the detected mcfDNA in molecules per microliter (MPM). An additional assay for mcfDNA sequencing referred to as the Karius Discovery (KD) assay was applied to investigate microbial fragmentomics patterns further. The KD assay was designed to offer a more comprehensive survey of the mcfDNA, estimating microbial concentration across over 16,000 different microbial species.

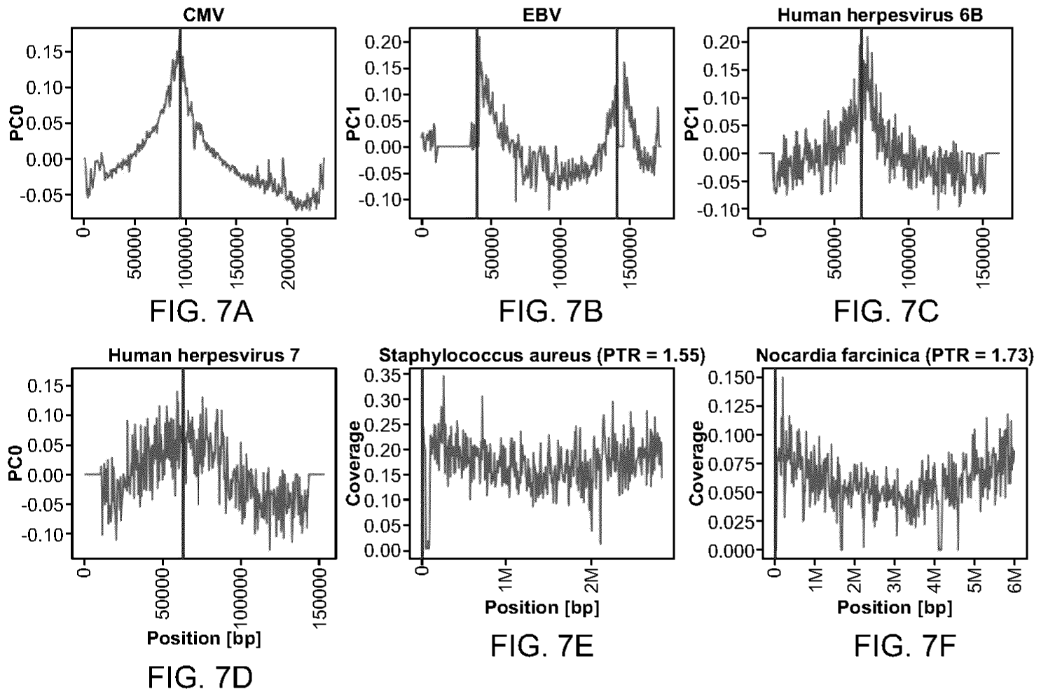

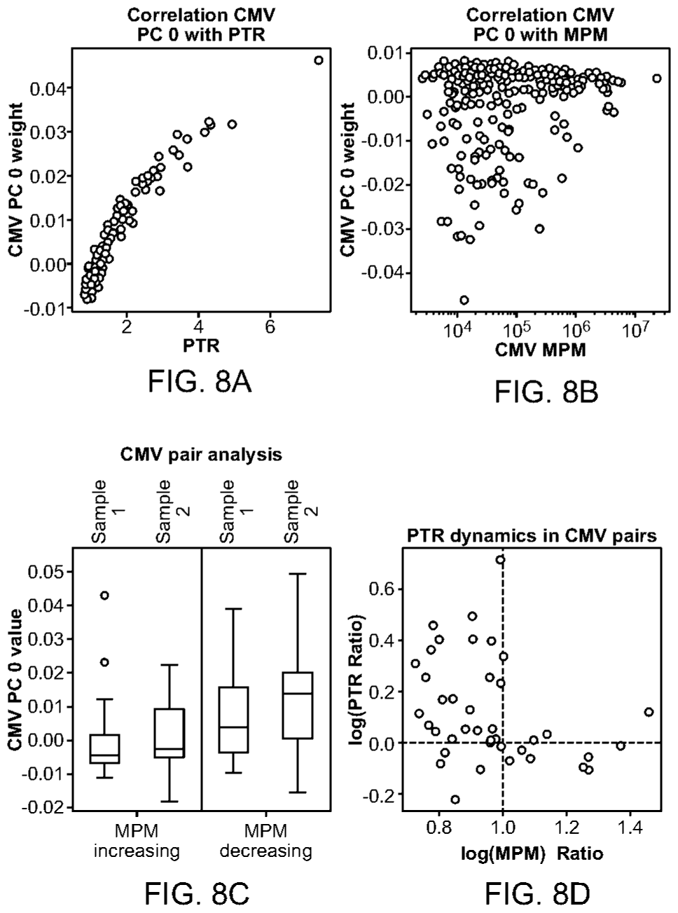

A broadly generalizable approach to infer the rate of DNA replication in contexts as diverse as human cell culture and bacterial infections is to compare whole genome sequencing coverage near the origin of replication to the coverage in later-replicating regions. For microbes with a single origin of replication (such as pCMV) the ratio of observed sequencing reads near the origin of replication to those near the terminus, denoted as a Peak (origin) to Trough Ratio (PTR), can provide an intuitive estimate of the replication rate. PTR analysis has not been previously used to infer viral replication from mcfDNA in a living host, therefore, an investigation was conducted into whether and to what extent sequencing biases were seen around the origin of replication in viral mcfDNA derived from 679 clinical samples processed with KT. First, principal component analysis (PCA) was used on microbial mcfDNA coverage profiles to identify sites with elevated coverage, potentially indicative of the replication origin. By inspecting the principal components (PC) associated with the highest eigenvalues, peak structures surrounding the origin of replication were identified in HHV-4 (EBV), HHV-5, HHV-6B and HHV-7 (FIGS. 7A-D), while no peak was observed for BK polyomavirus, HHV-2 or HHV-8. Similar coverage patterns surrounding the origin of replication were also observed in mcfDNA originating from bacteria (FIGS. 7E-F), albeit at a lower frequency. Projecting mcfDNA coverage profiles onto the PC most associated with the origin-of-replication peak exhibited a positive value, possibly indicative of active replication in 26.7% of 300 studied HHV-5 cases, 26.9% of 78 HHV-6B cases, and 23.1% of 13 HHV-7 cases. Finally, by projecting HHV-5 genomic coverage profiles onto the PC vectors, the weight of the PC most associated with the peak structure was found to be strongly correlated with the calculated PTR of the coverage profile (Spearman r=0.82, p=2.54e-74, FIG. 8A).

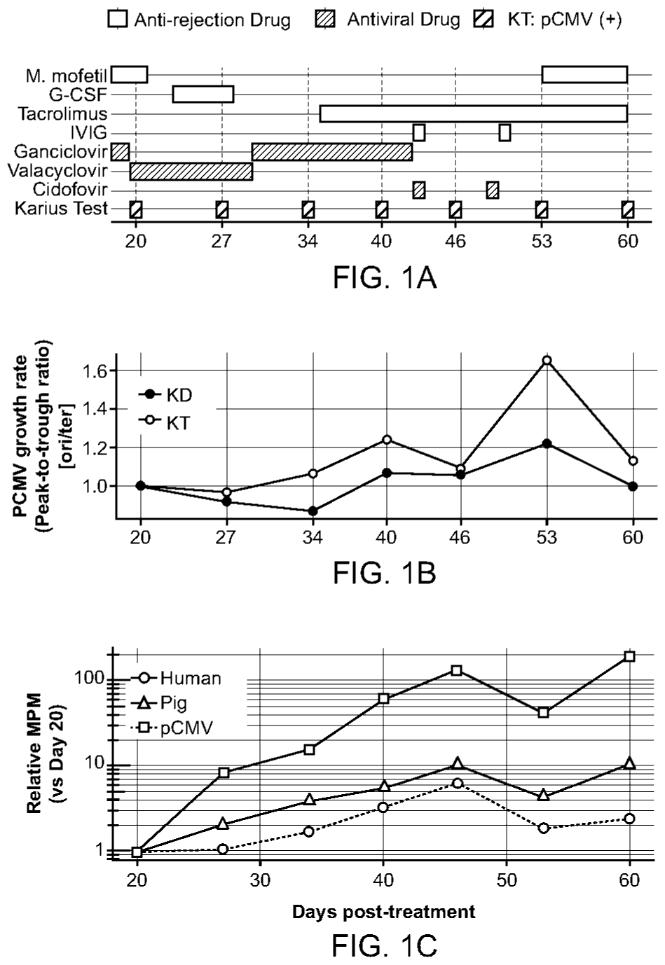

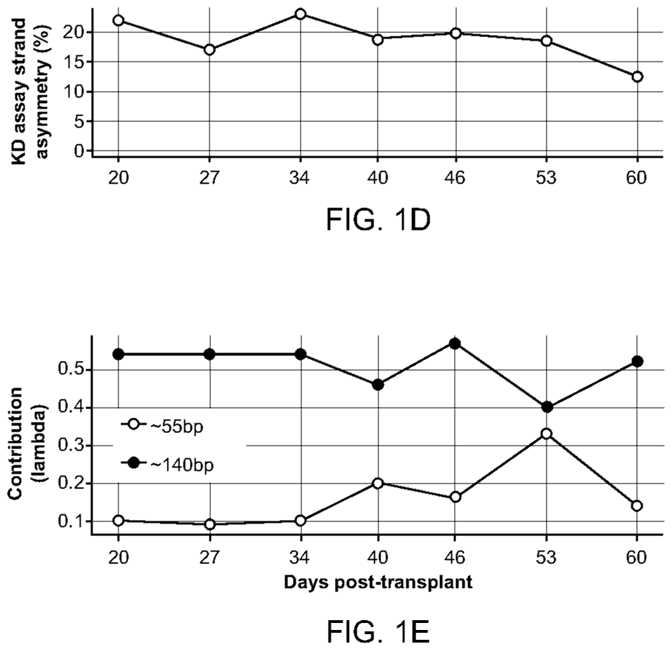

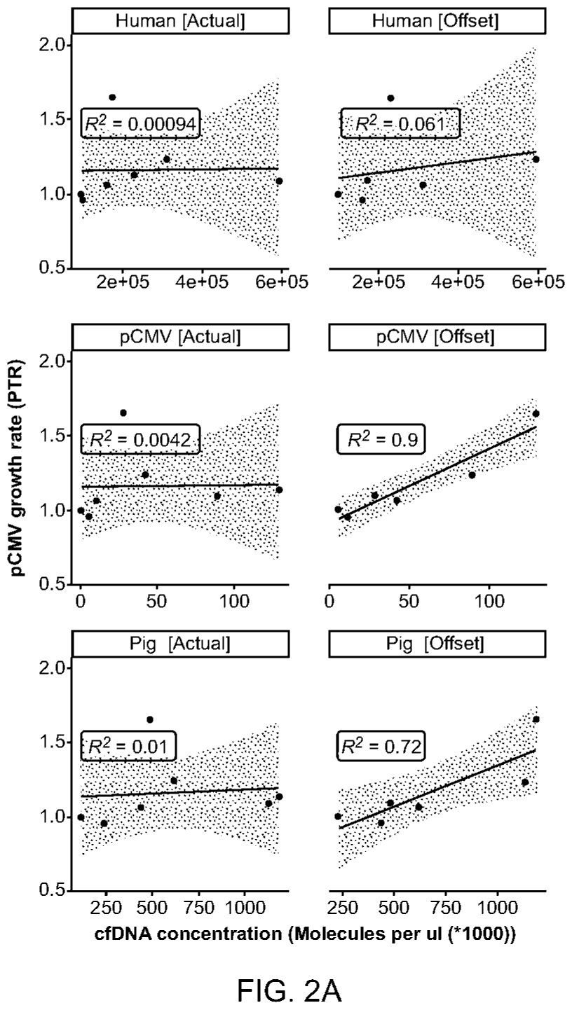

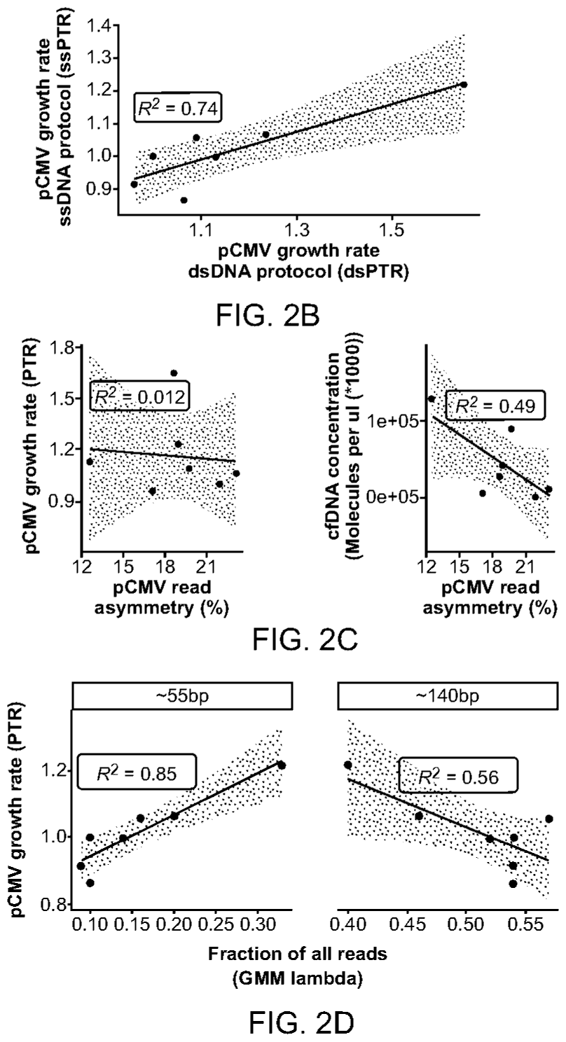

Using pCMV mcfDNA-sequencing reads (KT), the pCMV PTR was found to be increased up to Day 40, declined at Day 46, and then increased further to a maximum rate at Day 53 (FIG. 1B). The decline at Day 46 coincided with the initiation of a different replication-blocking antiviral treatment (cidofovir) three days prior (FIG. 1A). A strong positive correlation was noted between the PTR and the cfDNA concentration when offset by one timepoint (R²_{Offset}=0.90; FIG. 1B, FIG. 1C, FIG. 2A), potentially implying that pCMV mcfDNA concentrations are a readout of recent but not necessarily current viral replication rates. The extent of pCMV replication exhibited a weaker positive correlation with the offset concentration of porcine cfDNA (R²_{Actual}=0.01; R²_{Offset}=0.72; FIGS. 1B, IC, 2A), and a very low correlation with human cfDNA concentration (R²_{Actual}=0.001; R²_{Offset}=0.06; FIG. 1B, FIG. 1C, FIG. 2A). Such a reduction in correlation would be consistent with the death of more porcine cardiac cells as viral activity increased.

To assess if this type of relationship between putative replication signal and offset mcfDNA concentration is generalizable, a subset of 74 patient cases were identified from commercial KT samples in which a human herpesvirus related to pCMV (HHV-5) was detected in consecutive KT tests, taken<14 days apart were studied. The sample pairs were categorized as either increasing if the HHV-5 mcfDNA concentration increased between the first time point and the second time point or denoted as decreasing otherwise. The HHV-5 coverage profiles were then projected on the previously calculated HHV-5 PCs, and the corresponding weight of the single PC most associated with the peak was determined per sample. Interestingly, pairs categorized as increasing exhibited significantly lower projected weights (i.e., less pronounced peaks) in both the first (Mann-Whitney-U, n₁=30, N₂=44, p=0.0058) and the second time points (Mann-Whitney-U, N₁=30, N₂=44, p=0.0003) when compared to the matching time points coming from result pairs categorized as decreasing (see FIG. 7C). The more pronounced coverage peaks were observed in sample pairs with decreasing HHV-5 concentration (FIG. 8D), where the coverage patterns surrounding the peak became even more dominant at the second time point compared to the first time point.

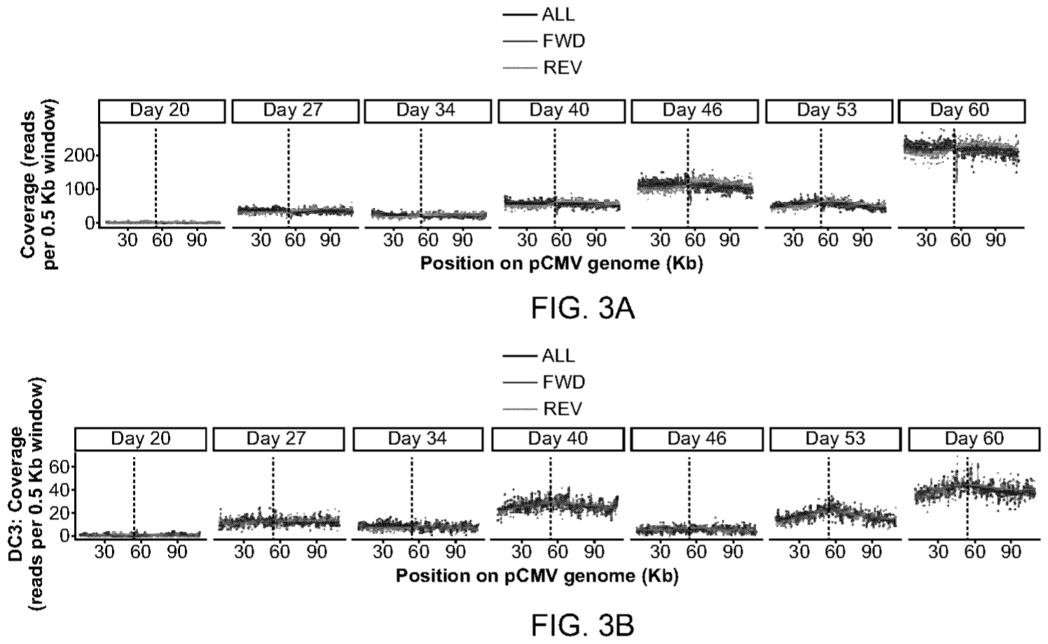

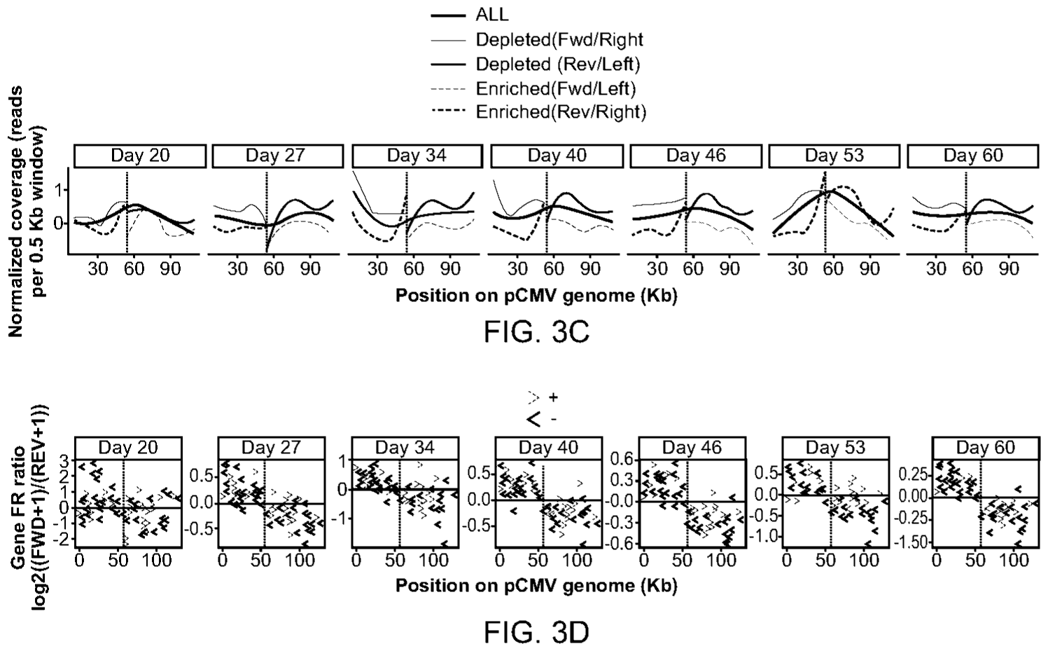

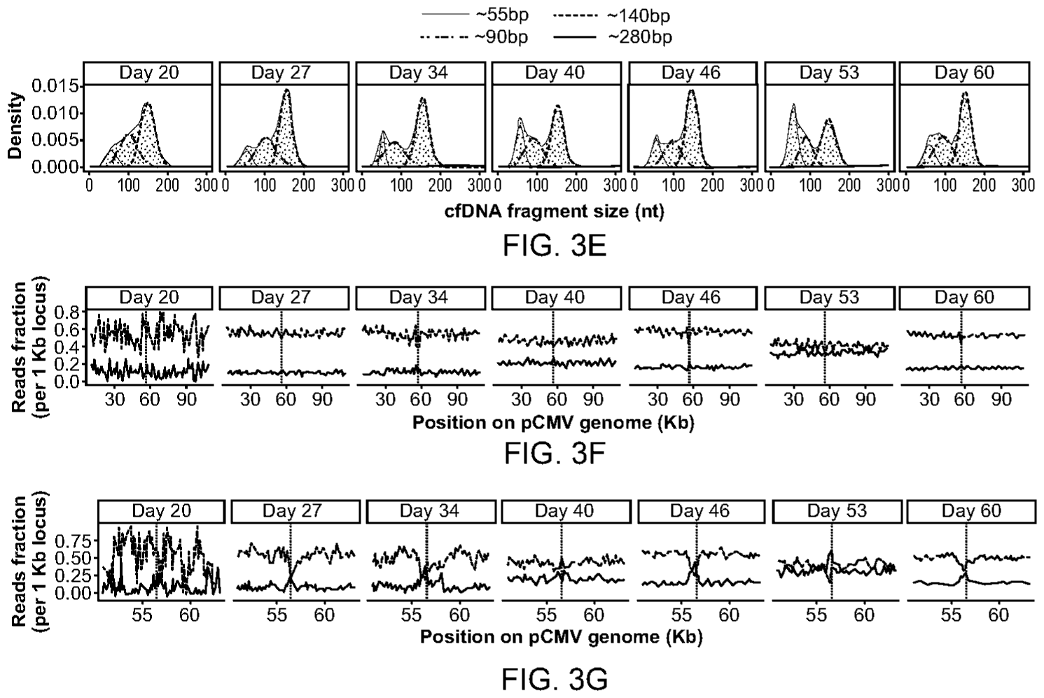

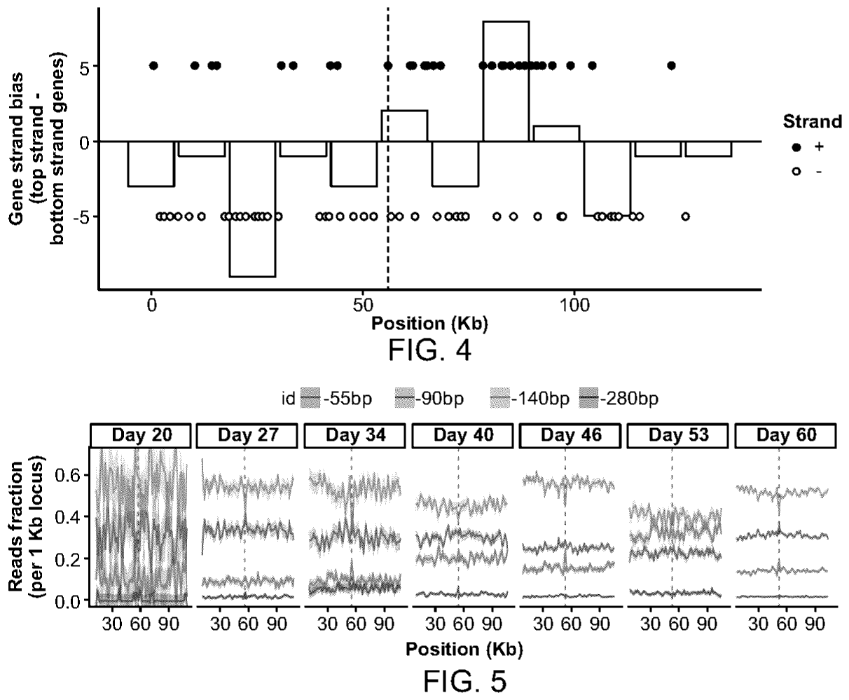

To explore the replication-associated signals in the xenotransplant patient in greater detail, the mcfDNA was quantified using an assay capable of detecting mcfDNA in both its double-stranded and single-stranded forms (denoted KD). Overall pCMV PTRs from KD mirrored those generated using KT (FIG. 1B, 2B). As pCMV is a double-stranded DNA virus, both DNA strands are equally likely to be represented in mcfDNA sequencing. Interestingly, at all time points, a bias was identified in the strand of origin of pCMV mcfDNA that changes polarity at the origin of replication (FIG. 3A, FIG. 3B). The magnitude of asymmetry was similar across all time-points (FIG. 1D) ranging from 23% (Day 20) to 12% (Day 60), extended from the origin to the terminus, and did not correlate with the pCMV PTRs (FIG. 1D, FIG. 2C). Replication governs many aspects of microbial genome structure, and gene locations exhibited pronounced strand asymmetry around the origin of replication in the pCMV genome (FIG. 4). Hence, it was hypothesized that the observed strand bias around the origin of replication might be associated with transcription, mcfDNA strand asymmetry did not seem to depend on gene polarity (FIG. 3C). These results suggested that the observed mcfDNA strand asymmetry may not be directly associated with transcription intermediates, and may instead represent the capture of steady-state ssDNA replication intermediates that do not increase in frequency during active replication. FIG. 2A-D shows pCMV PTRs correlated with the abundances of different fragment populations. FIG. 2A shows correlations between pCMV PTRs and cfDNA concentrations in plasma. The left panels show the correlation between values at the same time point (Actual). In the right panels, PTR values were instead compared to the MPM from the subsequent time point (Offset). FIG. 2B shows PTR values were correlated between the dsDNA-only and ssDNA protocols. FIG. 2C shows the extent of sequencing read asymmetry was not correlated with the inferred pCMV PTRs. It was negatively correlated with pCMV cfDNA concentration. FIG. 2D shows pCMV PTRs were positively correlated with the relative abundance of reads from the ˜55 nt population. Porcine CMV PTRs were negatively correlated with the relative abundance of reads from the ˜140 nt population. R²=squared Pearson correlation coefficients.

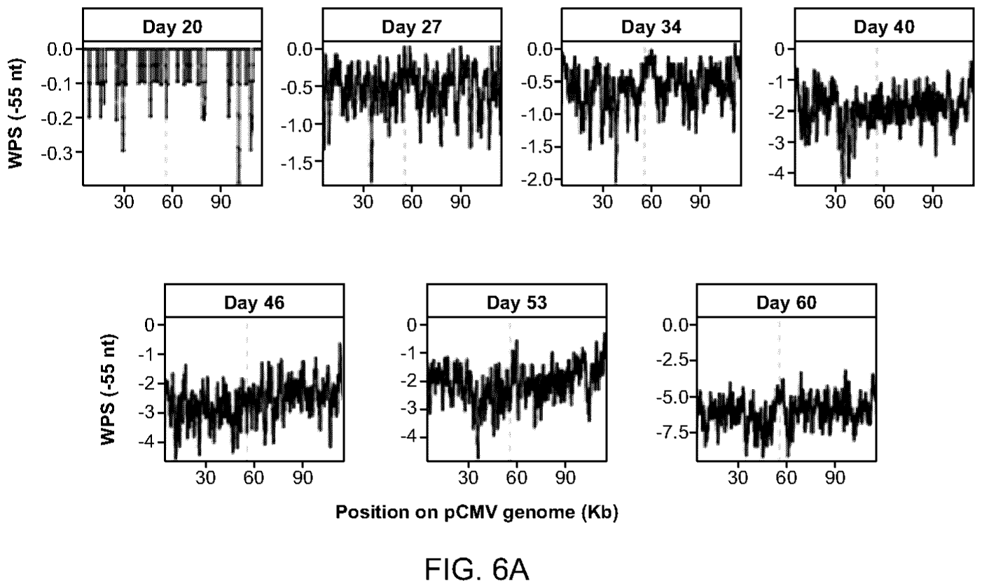

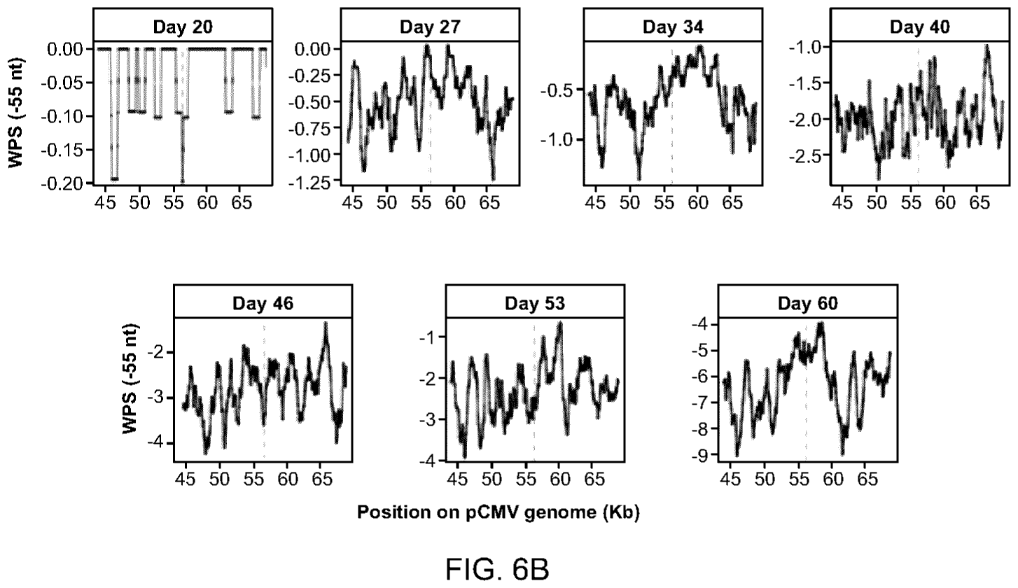

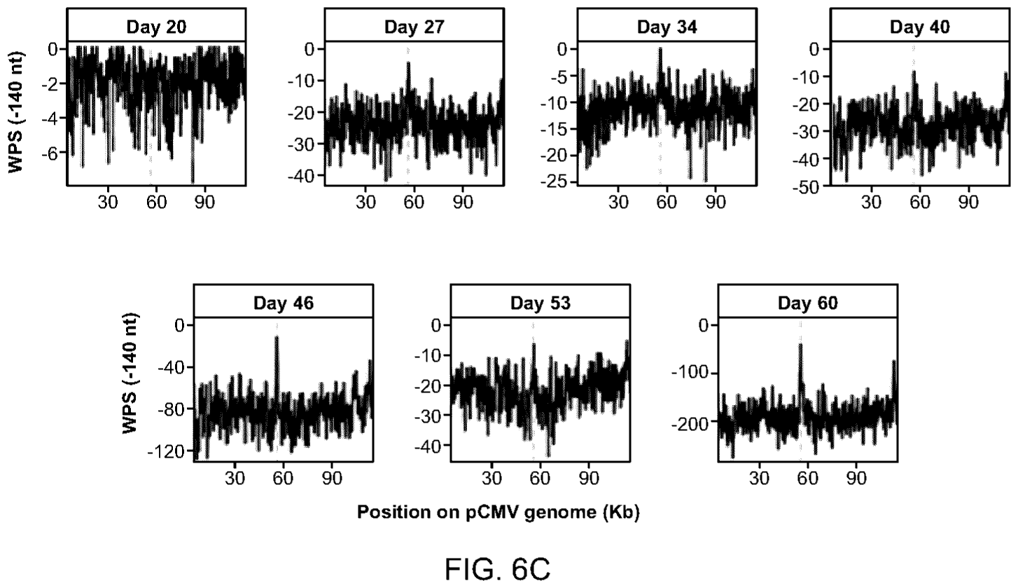

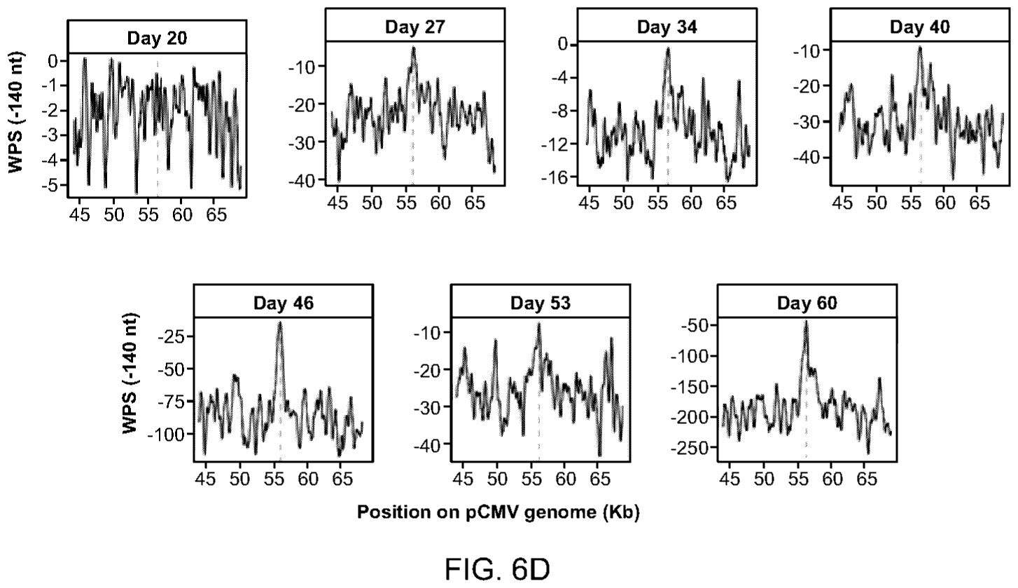

Next pCMV, human, and porcine cfDNA fragment sizes for pCMV were examined and a multi-modal distribution at all time points was found (FIG. 3D). In particular, by fitting a mixture of Gaussians to the observed mcfDNA fragment length distributions, four distinct populations of fragments were identified in each sample with mean lengths of ˜55 nt, ˜95 nt, ˜145 nt and ˜280 nt (FIG. 3E). The 55 nt, 145 nt and 280 nt populations may correspond to single-stranded DNA, mononucleosome-bound and dinucleosome-bound cfDNA, respectively. The contribution of each population fluctuated through the course of treatment. Of particular note for pCMV was the 3-fold increase in the proportion of sequencing reads from the ˜55 nt population from Day 20 to Day 56; this increase coincided with a decrease in the proportion of sequencing reads from the ˜145 nt population (FIG. 1E, FIG. 3G). Conversely, the proportion of fragments from the 95 nt and 280 nt populations remained relatively stable (FIG. 5). It was also observed that an increased contribution of 55 nt fragments on days 40 and 53 was seen for both the pCMV and human fractions. Although the trend of long to short fragments was observed to occur genome-wide, there was also an evident localized enrichment for shorter pCMV mcfDNA fragments near the origin of replication (FIG. 3F, FIG. 3G). The residence of proteins on DNA may have contributed to local changes in the fragment populations. To assess protein residence, a window-protection score (WPS) was calculated (FIGS. 6A-D). The WPS calculation was done for 55 bp fragments over the whole genome (FIG. 6A), 55 bp fragments over a 25 kb region centered at the pCMV origin of replication (FIG. 6B), 140 bp fragments over the whole genome (FIG. 6C), and 140 bp fragments over a 25 kb region centered on the origin of replication (FIG. 6D). The WPS calculations revealed a highly protected region in the 140 nt population centered at the origin. FIG. 4 shows gene strand bias in the pCMV genome. There was a strong bias for bottom-strand genes to the left of the replication origin (dashed line). Most top-strand genes were to the right of the origin, however, there is a less pronounced top/bottom bias on this side. Each gene is indicated as a dot (+ strand, − strand). Bars indicate the number of top-bottom strand genes in a 12 Kb window.

The application of PTR scores to determine replication was well-suited to scenarios in which a single origin of replication was identified. To support a broader de-novo identification of replication signals, as well as extend the methodology to support multiple replication initiation sites, a principal component analysis (PCA) based methodology was applied. In particular, PCA was performed on normalized microbial genomic coverage profiles, and the principal components (PC) associated with the highest eigenvalues were inspected. Using this methodology, the analysis of coverage patterns was expanded to include a broader set of 679 clinical samples processed with KT, restricting to taxa that exhibited sufficiently high concentrations supporting genome-wide coverage analysis. As expected, when projecting CMV genomic coverage profiles onto the PC vectors, the weight of the PC most associated with the peak structure was found to be strongly correlated with the calculated PTR of the coverage profile (Spearman r=0.82, p=2.54e-74). The weight of the corresponding PC was not correlated with the overall CMV concentration (Spearman r=0.02, p=0.73). When expanding the analysis to other viruses and bacteria, similar mcfDNA coverage patterns surrounding the origin of replication for some of the examined taxa was noted. Peak structures surrounding the origin of replications were identified among the top three PCs in CMV, EBV, Human herpesvirus 6B, and Human herpesvirus 7, while no peak was observed for BK polyomavirus, Human herpesvirus 2, and Human herpesvirus 8. Projecting mcfDNA coverage profiles onto the PC most associated with the origin-of-replication peak exhibited a positive value in 26.7% of 300 studied CMV cases, 26.9% of 78 HHV-6B cases, and 23.1% of 13 HHV-7 cases. Similar coverage patterns surrounding the origin of replication were also observed in mcfDNA originating from bacteria, albeit at a lower frequency. The multi-contig genome reference structures, often associated with bacterial genome assemblies, may have limited the ability to assess bacterial fragmentomics when compared to viral ones.

A detailed analysis was performed of the cfDNA populations found in the plasma of the first human to receive a porcine heart xenotransplant, with a particular focus on the mcfDNA of pCMV. Broadly, cfDNA has demonstrated its value in the study of transplantation outcomes, as it allows the concurrent study of DNA from the host, from the transplanted organ, and from the microbial community that may impact the patient's outcome.

The cfDNA populations found in the plasma of the first human to receive a porcine heart xenotransplant were analyzed, focusing on the mcfDNA of pCMV. By examining the mcfDNA present in the patient's plasma over the 60 days, from transplantation to death, evidence was found to suggest that a porcine virus, pCMV, was actively replicating during the later stages of this patient's treatment. Latent pCMV is known to reactivate during pig-to-primate transplantations, and has been shown to detrimentally affect transplanted organs in baboons; however, it remains unknown if pCMV detrimentally affected the porcine xenograft in this case. A large uptick was found in pCMV mcfDNA derived from genomic loci near the origin of replication coincided with the deterioration of the patient. In bacteria, elevated sequencing coverage at the origin of replication is known to correlate with replication rates. Similarly, in humans, early and late replicating regions of the genome are commonly inferred from differences in sequencing coverage depth (REFs).

The extent of pCMV replication did not consistently increase over time, as may have been expected for a rapidly growing virus; however, the patient's regimen of antiviral medications may have influenced this trajectory, as all three antiviral medications administered act by inhibiting viral replication (FIG. 1A). Ganciclovir is the gold standard therapy for human CMV, and was administered from Day 0 to 19 as a prophylactic. The initial appearance of pCMV mcfDNA (day 20) coincided with a switch from ganciclovir to valacyclovir between days 19 and 30. Porcine CMV indicators of replication increased from day 34 to 40, despite the resumption of ganciclovir treatment, then abruptly decreased when the patient was switched from ganciclovir to cidofovir at day 43. Porcine CMV has previously been reported to have reduced susceptibility to ganciclovir, and this change in treatment from ganciclovir to cidofovir, corresponded with an improvement in the patient. The strongest replication signal was observed at Day 53, despite continued cidofovir treatment.

Although the extent of the pCMV replication signal was not correlated with the pCMV mcfDNA concentration within a sample, there was a strong correlation with mcfDNA concentration at the subsequent time-point, approximately seven days later. The strongest replication signal was observed on Day 53, at the time-point that immediately followed the peak pCMV mcfDNA concentration. An analogous correlation was not seen in the 74 HHV-5-positive cases from KT data with paired time points. Importantly, however, is the lack of clinical details of the patients from whom these samples were taken; thus, heterogeneity of treatment regimens, reporting, and overall patient statuses among these paired samples are likely confounding the interpretation. A well-controlled time series study must follow to investigate the varying types of observed correlations in greater detail. A correlation was observed between pCMV replication and porcine cfDNA concentration. The correlation could arise from the death of more porcine cardiac cells as viral activity increased; however, given the limited number of data points, such a correlation may also be unrelated to pCMV replication and simply reflect coincident deterioration in the xenograft tissue.

In addition to increased coverage, a clear mcfDNA strand asymmetry was also identified around the pCMV origin of replication. One hypothesis is that this asymmetry represents the capture of single-stranded DNA intermediates of DNA replication. Gene orientation is also highly asymmetric around the origin of replication; although no evidence was found that ssDNA intermediates of transcription were contributing to the observed strand asymmetry, the limited knowledge of pCMV biology means that it cannot be fully ruled out. Thus, the source of cfDNA strand asymmetry around the pCMV origin remains enigmatic.

Microbial cell-free DNA contains different populations of DNA fragments, likely derived from a range of biological processes. Viruses with different life-cycle strategies may yield mcfDNA of different sizes, with shorter (<100 nt) cfDNA likely associated with a proliferative state. Of particular interest was a population of ˜55 nt mcfDNAs, previously seen in human nuclear, human mitochondrial and microbial cfDNA, and the population of nucleosome-sized mcfDNA fragments. pCMV cfDNA derived from the 55 nt population were found to progressively increase through the treatment regimen at the expense of nucleosome-size fragments, and a similar trend was not observed in porcine cfDNA. One hypothesis to explain this concerted change in the human and viral cfDNA populations is that a single biological process governed the progressively shifting distribution of the cfDNA fragment sizes; for example, a host immune response to pCMV-infected human cells. It is also possible that a fraction of the observed shift in fragment lengths may have been associated with a change in viral biology, as a localized change was observed in fragment populations at the viral origin of replication. To this end, short cfDNAs are known to coincide with putative non-canonical structures implicated in the regulation of DNA replication. A possibility is that the two fragment populations derive from viruses at different stages of their life cycle; the DNA of HHV-6A and HHV-6B, the closest known relatives of pCMV, is wrapped by nucleosomes when the virus integrates into the host genome. Thus, longer fragments may represent host-integrated and latent viral DNA, whereas the shorter fragments may be associated with an active, replicating state. The fraction of short pCMV fragments was maximal at Day 53. Interestingly, the increase in short pCMV fragments coincides with the appearance of a notably large population of ˜55 nt fragments in the human-derived cfDNA. One hypothesis to explain the concerted change in the short human and viral cfDNA populations is that the non-specific nucleolytic activity of a host immune response to pCMV-infected human cells yielded short mcfDNA. While it remains unknown whether pCMV can infect human cells in vivo, peripheral blood mononuclear cells from this patient did test positive for pCMV1. Alternatively, it is possible that there was a coincident proliferation of both pCMV and human immune cells at this time point. Independent of the source of the 55 nt fragments, the relative fraction of 55 nt fragments was correlated with changes in viral replication signals and demonstrated elevated frequencies surrounding the origin of replication in pCMV across several time points. The dynamic nature of the mcfDNA fragment length profile may also be an important consideration when quantifying viral load for clinical interpretation; for example, qPCR-based tests to quantify human CMV yield highly variable findings, which may be the result of the amplicon length varying from 50-350 bp among assays. The data suggest that shorter amplicons would be more likely to yield reproducible estimates of viral load.

Microbial fragmentomics may provide novel biomarkers for infectious disease diagnosis and prognosis, for understanding microbe-immune interactions, and for studying the response to therapy. Elevated coverage surrounding the origin of replication, diversity of fragment lengths, locations, composition, as well as strand biases have the potential to inform replication kinetics, which may then be correlated with the severity and progression of disease. In the case of the pCMV detection in the first porcine-to-human cardiac xenotransplantation, this work provided data supporting the active replication of the pCMV. It remains to be determined whether pCMV proliferation was responsible for the ultimate failure of the xenotransplanted heart.

### Methods

### Antiviral Treatment Regimen

Ganciclovir, a drug with limited efficacy against pCMV at standard doses was administered from Day 0 to 19 for antiviral prophylaxis but at a higher dose, even when adjusted for the patient's reduced creatinine clearance. The initial appearance of pCMV mcfDNA (day 20) coincided with a switch from high-dose ganciclovir to valacyclovir (not thought to be active against pCMV) between days 19 and 30. This switch was prompted by concerns of bone marrow toxicity from high-dose ganciclovir. Ganciclovir was resumed on day 30 as pCMV mcfDNA was beginning to increase, but at a lower dose than previously prescribed, due to earlier concerns related to drug-induced toxicities. Porcine CMV signals that may be associated with replication increased from day 34 to 40, despite the resumption of ganciclovir prophylactic treatment, then abruptly decreased when the patient was switched from ganciclovir to cidofovir at day 43. Cidofovir has been demonstrated to reduce pCMV viral loads in cells in vitro. Although pCMV levels increased on day 60, this increase occurred in the setting of augmented immunosuppression with rituximab and complement inhibitors as well as plasma exchange for the treatment of suspected xenograft rejection.

### Paired-End Alignment to Porcine CMV Genome

KD libraries were sequenced in paired-end mode (56 bp read 1, 20 bp read 2) on an Illumina NextSeq. The first read of every sequencing read pair was aligned to the human GRCh38 reference genome, the human GRCh38 decoys, and the porcine Sus scrofa 10.2 reference genome using bowtie2 2.4.5. Sequencing reads that did not align to any of these genomic references were retained. Sequencing read pairs were then subjected to adapter and quality trimming using fastp 0.20.1 (−length_required 19−cut_front 3−cut_tail 3) and were subsequently aligned to the Suid herpesvirus 2 (porcine CMV) reference genome (accession: GCA_000913455.1) using bowtie2 2.4.5 (−very-sensitive-local) in paired-end mode.

### Read Processing

Duplicate reads were flagged using samtools and fragment lengths were calculated using the pysam API for python. Fragment orientation was inferred from the orientation of the first read of each sequencing read pair. Populations of fragments were inferred at each time-point using a Gaussian Mixture Model (GMM) fit using Expectation-Maximization (EM) (R mixtools package; normalmixEM function). The EM was initialized with manual initial estimates of the mean fragment length (mu; 55,90,140,280) and standard deviations (sigma; 1,8,8,8) for the 55 nt, 100 nt, 140 nt and 280 nt populations, respectively. GMMs were also fit using all combinations of 2 or 3 of these populations; however, these did not fit the observed fragment-length distribution as well as the 4-population GMM. Fragments were partially attributed to each population based on fragment length.

### Coverage Analysis of Karius Test Clinical Samples

### Selection of Taxa and Test Results

While multiple, competing genomes in the Karius Test (KT) pathogen database improved the overall sensitivity of the assay, they may have had a negative impact on the analysis of genome coverage due to shared read alignments between the assemblies. Furthermore, highly fragmented, unscaffolded genome assemblies limited the ability to robustly compute meaningful coverage metrics. Therefore, the 246 taxa identified were detectable by the KT that are represented by a single assembly in the database where the 5 longest contigs account for more than 95% of the total sequence length.

Among over 30,000 KT results, only results where one of the identified taxa was detected by the KT pipeline and had at least 4000 reads assigned to it were selected. Only taxa with more than 10 occurrences were considered for further analysis.

### Computation of Coverage Vectors and PCA

For each genome, only the longest contig was used to compute the genome coverage. The contig was binned into 400 bins, and the number of nucleotides aligned to each bin was counted and divided by the bin width. Binned coverages were transformed into coverage vectors by normalizing bin values to sum up to 1. For each taxon, Principal Component Analysis (PCA) of the coverage vectors was performed. The first 10 principal components (PC) were visualized and manually inspected for non-uniform patterns around the ORI. For several microbes of interest, PCs with peak-shaped coverage patterns around the ORI (FIG. 7A-F).

### HHV-5 Peak PC and Peak-to-Trough Ratio

For human cytomegalovirus (HHV-5), the first PC (PC0, accounting for 39.7% of the variance) had an evident peak shape coverage surrounding the origin of replication (FIG. 7A). Therefore, when projecting 300 high-coverage HHV-5 sequencing profiles on the computed PCs, the weight of PC0 was used as a quantitative measurement of the peak size. Visual inspection confirmed that a noticeable peak shape around the ORI was only visible for HHV-5 PC0 weight>0. Peak-to-Trough Ratio (PTR) was calculated for HHV-5 as the ratio between the median coverage around the ORI (positions 85,000-105,000) and the median of the coverage at both sides of the peak (making sure to not include coverage artifacts, positions 20000-30000 and 150000-160000) based on genome sequence NC_006273.2. FIG. 7A, FIG. 7B, FIG. 7C, and FIG. 7D show the principal components exhibiting the most pronounced peak structure across four different viruses. FIG. 7E and FIG. 7F show coverage profiles of two bacteria with circular genomes, Staphylococcus aureus and Nocardia farcinica, as studied in two independent cases, respectively. The vertical lines highlight the approximate location of the origin of replication.

A strong correlation was shown between the HHV-5 PC0 weight and the corresponding genome PTR (Spearman r=0.820, p=2.54e-74), suggesting that both measures can be used exchangeably for measuring peak size (FIG. 8A). The presence and size of the peak shown did not correlate with the overall HHV-5 cfDNA concentration in blood (FIG. 8B) (Spearman r=0.019, p=0.73).

### Analysis of Paired HHV-5 Samples

The Karius Test data was screened for pairs of test results according to the following criteria: (a) the same patient case, (b) samples processed less than 14 days apart, (c) HHV-5 detected by mcfDNA sequencing in both samples, and (d) at least one HHV-5 detection had more than 100 sequencing reads. In total 74 sample pairs matched the criteria, and HHV-5 coverage vectors were computed for all results. Sample pairs were categorized into increasing or decreasing based on the HHV-5 concentration change over time. In total, 30 result pairs had increasing HHV-5 MPM and 44 result pairs had decreasing MPM. When looking at the peak size measured by the HHV-5 PC0 (see above), result pairs denoted as increasing had significantly smaller coverage peaks around the ORI when compared to pairs denoted as decreasing (FIG. 8C) under the Mann-Whitney U test; the observation held true for both the peak size in the first result (p=0.0058) and the second result (p=0.0003) of the pair. A closer look into the coverage peak dynamics observed in result pairs shows that increasing peak sizes (as measured by a PTR Ratio>1) are almost exclusively observed in sample pairs where the HHV-5 concentration decreases (FIG. 8D).

### Example 2: Donor Fragmentomics Indicates Post-Xenotransplant Rejection

A bovine-to-human hepatic xenotransplantation is performed on a 23-year-old woman with severe liver disease. The subject is monitored over a 60-day time course. An evident increase in bovine cytomegalovirus (bCMV) concentration is identified around 22 days post-xenotransplant based on the observation of non-uniform microbial cell-free DNA (mcfDNA) surrounding the origin of replication along with the emergence of shorter mcfDNA fragments suggesting that bCMV is likely to be actively replicating. However, a decrease in bCMV concentration is identified around 5 weeks post-xenotransplant based on mcfDNA surrounding the termini of the bCMV genome suggesting that bCMV is likely to no longer be replicating. Despite the decrease in bCMV concentration at 30 days post-xenotransplant, the patient is continuing to decline. Further analysis of cell-free DNA (cfDNA) from the plasma of the patient identifies high concentrations of bovine cfDNA. The elevated bovine cfDNA is likely a reflection of increased xenograft rejection.

Plasma mcfDNA sequencing is performed in an accredited laboratory referred to herein as the Karius Test (KT), to provide unbiased testing for human pathogens. The assay is designed to report the absolute concentration of detected mcfDNA in molecules per microliter (MPM). An additional assay for mcfDNA sequencing referred to as the Karius Discovery (KD) assay is applied to investigate microbial fragmentomics patterns further. The KD assay is designed to offer a more comprehensive survey of the mcfDNA, estimating microbial concentration across over 16,000 different microbial species.

A broadly generalizable approach to infer the rate of DNA replication in contexts as diverse as human cell culture and bacterial infections is to compare whole genome sequencing coverage near the origin of replication to the coverage in later-replicating regions. For microbes with a single origin of replication (such as bCMV) the ratio of observed sequencing reads near the origin of replication to those near the terminus, denoted as a Peak (origin) to Trough Ratio (PTR), can provide an intuitive estimate of the replication rate.

Using bCMV mcfDNA-sequencing reads (KT), the bCMV PTR is found to be increasing from identification of bCMV mcfDNA in plasma samples of the patient collected at Day 10 up to Day 22 and then declining at Day 30. The decline at Day 30 of bCMV mcfDNA coincides with the initiation of a different replication-blocking antiviral treatment (cidofovir) three days prior (Day 27). A strong positive correlation is noted between the PTR and the cfDNA concentration when offset by one timepoint potentially implying that bCMV mcfDNA concentrations are a readout of recent but not necessarily current viral replication rates.

Following the decrease in bCMV mcfDNA concentration 30 days post-transplant, the concentration of bovine cfDNA from plasma of the subject continues to increase. The increase in bovine cfDNA in the plasma of the subject indicates the bovine liver is being rejected.

### Example 3: Donor Fragmentomics Indicates Allotransplant Rejection

A human-to-human lung allotransplantation is performed on a 47-year-old man. The subject is monitored over a 60-day time course. There is no increase in microbe concentration detected over the longitudinal analysis of plasma samples from the subject. Despite the absence of microbe concentrations indicative of microbial replication and an active infection based on comparisons of microbial concentrations collected over the first 30 days post-xenotransplant, the patient is continuing to decline.

Further analysis of cell-free DNA (cfDNA) from the plasma of the patient to analyze the human cfDNA is performed. To distinguish between the donor (transplant-derived) cfDNA and the subject (host-derived) cfDNA, the cfDNA is analyzed for single-nucleotide variant and cell-type specific patterns of genome coverage. This analysis identifies elevated donor (transplant-derived) cfDNA which is likely a reflection of increased allotransplant rejection.

It should be understood that the subject matter defined in the appended claims is not necessarily limited to the specific implementations described above. The specific implementations described above are disclosed as examples only.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method comprising:
(a) providing a cell-free biological sample from a human subject, wherein a microbe is present in the human subject or suspected of being present in the human subject;
(b) performing high-throughput sequencing on nucleic acids in the cell-free biological sample to generate microbial cell-free nucleic acid (mcfNA) sequence reads;
(c) aligning the mcfNA sequence reads with a reference sequence containing sequences within an origin of replication region of the microbial genome to determine coverage of the mcfNA sequence reads within the origin of replication region of the microbial genome; and
(d) determining whether the microbe is actively replicating in the human subject based on the coverage of the mcfNA sequence reads within the origin of replication region of the microbial genome.

2. The method of claim 1, further comprising comparing the coverage of the mcfNA within the origin of replication region of the microbial genome to coverage in a later replicating region of the microbial genome to derive a peak-to-trough ratio (PTR) score.

3. The method of claim 2, wherein the determining in (d) comprises using the PTR score to determine whether the microbe is actively replicating in the human subject.

4.-10. (canceled)

11. The method of claim 1, wherein sequences within the origin of replication region of the microbial genome are within 0.5 kb, 1 kb, 1.5 kb, 2 kb, 5 kb, 8 kb, 10 kb, 15 kb, 20 kb, 25 kb, 30 kb or 50 kb of either side of the origin of replication and wherein the sequences either span or do not span the origin of replication.

12.-13. (canceled)

14. The method of claim 2, wherein active replication is identified when coverage within the origin of replication region of the microbial genome and coverage at a later replicating region are statistically different from each other and wherein the coverage at the origin of replication region of the microbial genome is higher compared to the coverage at the later replicating region.

15. The method of claim 3, wherein the PTR score is a statistically significant score and wherein active replication is identified when the PTR score is greater than 1.

16.-22. (canceled)

23. The method of claim 1, wherein the human subject is a transplant recipient.

24. The method of claim 1, wherein the human subject is a xenotransplant recipient.

25.-26. (canceled)

27. The method of claim 1, wherein the microbe is a bacterium or virus associated with a transplanted organ or graft or with a xenotransplanted organ or graft.

28. The method of claim 1, wherein the microbe is a virus.

29.-39. (canceled)

40. The method of claim 1, further comprising measuring lengths of nucleic acid fragments at the origin of replication region of the microbial genome.

41. The method of claim 1, further comprising determining gene strand bias of the bottom and top strands at the origin of replication region of the microbial genome.

42.-50. (canceled)

51. The method of claim 1, further comprising measuring principal components (PCs) of longest contiguous reads.

52. The method of claim 51, further comprising comparing PCs to a PTR score.

53. The method of claim 1, further comprising identifying a disease, disorder, or condition in the human subject wherein the disease, disorder, or condition is selected from the group consisting of: xenograft injury, transplant rejection, active viral infection, and latent viral infection.

54. The method of claim 1, further comprising administering a treatment to the human subject to treat a microbial infection identified by the method.

55.-57. (canceled)

58. The method of claim 1, wherein the method further comprises detecting donor-derived cell-free DNA in the transplant recipient.

59.-61. (canceled)

62. The method of claim 1, wherein the cell-free biological sample is a plasma sample, a serum sample, a blood sample, a saliva sample, a synovial fluid sample, a cerebrospinal fluid sample, or a urine sample.

63.-98. (canceled)
